(19) United States
(12) Patent Application Publication
(10) Pub. No.: US 2026/0347837 A1
(43) Pub. Date: Nov. 5, 2026

(54) GENOME-EDITED BIRDS

(71) Applicant: The State of Israel, Ministry of Agriculture & Rural Development Agricultural R, Rishon LeZion (IL)

(72) Inventors: Yuval CINNAMON, Jerusalem (IL); Enbal BEN-TAL COHEN, Rehovot (IL)

(21) Appl. No.: 19/550,573

(22) Filed: Feb. 26, 2026

Related U.S. Application Data
(63) Continuation-in-part of application No. 17/435,485, filed on Mar. 3, 2020, now Pat. No. 12,565,664.
(63) Continuation-in-part of application No. 16/647,474, filed on Sep. 17, 2018, now abandoned.
(60) Provisional application No. 62/560,218, filed on Sep. 19, 2017.
(60) Provisional application No. 62/814,162, filed on Mar. 5, 2019.

(51) Int. Cl. C12N 15/11; C07K 14/34; C07K 14/47; C12N 9/22; C12N 9/64; C12N 15/85; C12N 15/90

(52) U.S. Cl. CPC C12N 15/111; C07K 14/34; C07K 14/4703; C12N 9/222; C12N 9/6472; C12N 15/85; C12N 15/907; C12N 2310/20; C12N 2830/002; C12Y 304/22056

(57) ABSTRACT

The present disclosure provides DNA editing agents and exogenous polynucleotide cassettes for generating chimeric bird cells and chimeric birds. The polynucleotide cassettes can be used to produce conditionally-lethal phenotype in male bird embryos. In one embodiment, the present disclosure provides methods of editing DNA, using the DNA editing agents and exogenous polynucleotide cassettes.

### SEQUENCE LISTING STATEMENT

The nucleotide and amino acid sequences provided herewith are shown using standard letter abbreviations for nucleotide bases and amino acids as defined in 37 CFR 1.831 through 37 CFR 1.835. Only one strand of each nucleic acid sequence is shown, but the complementary strand is understood as included by any reference to the displayed strand. The Sequence Listing is submitted as an XML file named 113NEX02US02-SEQ.xml, 421,969 bytes, created Feb. 26, 2026, the contents of which are incorporated by reference herein in their entirety.

### TECHNICAL FIELD

The present disclosure relates to DNA editing agents, and their use in preparing DNA-edited cells and birds. The present disclosure further relates to methods of conferring a conditionally-lethal phenotype to male embryos in eggs of DNA-edited birds.

### BACKGROUND

The ZW sex-determination system is a chromosomal system that determines the sex of offspring in birds, some fish and crustaceans, some insects, and some reptiles. The letters Z and W are used to distinguish this system from the XY sex-determination system. In the ZW system, the ovum determines the sex of the offspring. Males are the homogametic sex (ZZ), while females are the heterogametic sex (ZW). The Z chromosome is larger and has more genes, like the X chromosome in the XY system.

Early gender identification and separation are important aspects of all avian commercial applications, in particular in the edible eggs industry. For broilers and turkeys, gender separation allows a better suited management and feeding schemes according to the different needs of the two genders. Essentially all commercial hatcheries use gender separation of flocks. Male chickens, which are of lower commercial value, are culled at the hatchery, whereas female chickens are used for egg production.

Currently, there are three methods available for determining the gender of poultry. The gender of day-old chicks can be determined either by vent/cloaca identification, or by feather-typing methods. Alternatively, male and female chicks can be grown together until secondary gender characteristics become apparent, then the chicks can be separated based on gender. Vent/cloaca typing relies on the appearance of gender-related anatomical structures. Feather typing is based on feather characteristics that differ between male and female chicks, for example down color pattern, and rapid/slow rate of growth of the wing feathers. The third method relies on the appearance of natural secondary gender characteristics, for example in males the combs and wattles become larger than those on females.

The vent/cloaca gender determination of day-old chicks is difficult and expensive. Identifying the gender of a bird requires highly skilled personnel. While easier to perform, feather typing has the disadvantage of being limited to specific genetic crosses of birds. Gender typing by secondary gender characteristics is the easiest method to perform but has the disadvantage of requiring birds of both genders to be grown together for the first weeks after hatching, which because of feed costs and feed conversion considerations can be more expensive to the hatchery than the expense of vent/cloaca typing.

Most importantly, only in the US and Europe, almost a billion of male chicks are destroyed by different methods every year. This is not only an economic problem, but it increasingly becomes an ethical problem.

There is a need in the field of commercial hatcheries for high-throughput methods for preventing the production of male chicks, preferably even at the egg stage, thus avoiding the problems associated with viable male chicks.

### SUMMARY OF INVENTION

Provided here is a technology, including compositions and methods, for in-ovo destruction of male embryo chicks. Such compositions and methods are beneficial as they allow a person skilled in the field of bird propagation, such as a farmer, to skew the natural 1:1 ratio between males and female offspring in favor of the more commercially-useful female offspring.

In one embodiment, provided herein is a DNA editing agent comprising a polynucleotide cassette having a formula 5′-LHA (left homology arm)-OIE (optogenetic-inducible element)-LIE (lethality-inducing element)-RHA (right homology arm)-3′ or a formula 5′-LHA-LIE-OIE-RHA-3′, wherein (i) the LHA comprises a first nucleotide sequence that is substantially homologous to a first corresponding nucleotide sequence on chromosome Z of a bird; (ii) the OIE comprises a first promoter functionally linked to a second nucleotide sequence encoding an inducer-activated site-specific recombinase enzyme; (iii) the LIE comprises a third nucleotide sequence encoding a lethality-promoting protein, which is operatively linked to the activity of the inducer-activated site-specific recombinase enzyme; and (iv) the RHA comprises a fourth nucleotide sequence that is substantially homologous to a second corresponding nucleotide sequence on chromosome Z of a bird.

In certain embodiments, one or both of the LHA and RHA are substantially homologous to a corresponding nucleotide sequence located in an openly transcribed region on chromosome Z of a bird. For example, the openly transcribed region can be located at or downstream to the histidine triad nucleotide binding protein 1-Z (HINT1Z) locus on chromosome Z of a bird.

In certain embodiments, the inducer-activated site-specific recombinase enzyme can be Cre recombinase (Cre) (SEQ ID NO: 113), or Mag (SEQ ID NO: 114 and SEQ ID NO: 65). In certain embodiments, the expression of the inducer-activated site-specific recombinase enzyme is induced by an inducer. In certain embodiments, the inducer is electromagnetic energy. For example, the inducer can be blue light having a wavelength of 450-485 nm.

In certain embodiments, the inducer-activated site-specific recombinase enzyme comprises non-functional peptide fragments of an inducer-activated site-specific recombinase that combine to form an active inducer-activated site-specific recombinase enzyme in the presence of the inducer.

In certain embodiments, the lethality-inducing protein mentioned herein can be a toxin, a pro-apoptotic protein, an inhibitor of the Wingless/Integrated (Wnt) signaling pathway, a bone morphogenetic protein (BMP) antagonist, a fibroblast growth factor (FGF) antagonist, a wild type Caspase 3, a constitutively active Caspase 3, Noggin, or a lethality-inducing fragment of any of the above proteins.

In one embodiment, the DNA editing agent disclosed herein comprises (i) a LHA comprising the sequence of SEQ ID NO:105, (ii) an OIE comprising the sequence of SEQ ID NO:100, which is connected to the sequence of SEQ ID NO:116, which is connected to the sequence of SEQ ID NO:101, which is connected to the sequence of SEQ ID NO:103, which is connected to the sequence of SEQ ID NO:102, which is connected to the sequence of SEQ ID NO:104, which is connected to the sequence of SEQ ID NO:116, or the OIE comprises the sequence of SEQ ID NO:100, which is connected to the sequence of SEQ ID NO:116, which is connected to the sequence of SEQ ID NO:102, which is connected to the sequence of SEQ ID NO:103, which is connected to the sequence of SEQ ID NO:101, which is connected to the sequence of SEQ ID NO:104, which is connected to the sequence of SEQ ID NO:116, or the OIE comprises the sequence of SEQ ID NO:100, which is connected to the sequence of SEQ ID NO:116, which is connected to the sequence of SEQ ID NO:107, which is connected to the sequence of SEQ ID NO:103, which is connected to the sequence of SEQ ID NO:108, which is connected to the sequence of SEQ ID NO:104, which is connected to the sequence of SEQ ID NO:116, or the OIE comprises the sequence of SEQ ID NO:100, which is connected to the sequence of SEQ ID NO:116, which is connected to the sequence of SEQ ID NO:108, which is connected to the sequence of SEQ ID NO:103, which is connected to the sequence of SEQ ID NO:107, which is connected to the sequence of SEQ ID NO:104, which is connected to the sequence of SEQ ID NO:116, (iii) a LIE comprising the sequence of SEQ ID NO:92, or SEQ ID NO:94, or SEQ ID NO:96, or SEQ ID NO:98, and (iv) a RHA comprising the sequence of SEQ ID NO: 106.

In certain embodiments, the polynucleotide cassettes disclosed herein can be applied to a bird such as a chicken, a turkey, a duck, a goose, a quail, a pheasant, or an ostrich.

In another embodiment, the present disclosure further provides bird cells that contain the polynucleotide cassettes disclosed herein. In another embodiment, there is provided a chimeric bird comprising bird cells that contain the polynucleotide cassettes disclosed herein.

Further provided, in another aspect, are methods of using the DNA editing agent disclosed herein to generate a chimeric bird. In certain embodiments, the methods comprise the steps of contacting the cells of a bird with the exogenous polynucleotide cassettes disclosed herein, thereby generating genome-edited bird cells, and then transferring these genome-edited bird cells to recipient bird embryos. Further provided, in another aspect, are chimeric birds generated from the above methods.

Further provided, in another aspect, is a method of inducing lethality in a male embryo of a bird, comprising the steps of administering the DNA editing agent disclosed herein to a population of bird cells, thereby generating genome-edited bird cells; transferring these genome-edited bird cells to recipient bird embryos; and exposing the embryos to an inducer that elicits expression of the lethality-promoting protein encoded by the DNA editing agent, thereby inducing lethality in male embryos of the bird.

In another embodiment, the DNA editing agent disclosed herein further comprises a safe-lock element inserted downstream to the promoter in the OIE but upstream of the sequence encoding the inducer-activated site-specific recombinase. The safe-lock element comprises nucleotide sequences (STOP element) that prevent transcription of the inducer-activated site-specific recombinase encoded by the OIE. In one embodiment, the STOP element is flanked by two FRT sites. In one embodiment, each of the DNA editing agents having the sequence of one of SEQ ID NOs:120-127 comprises a safe-lock element. Further provided, in another aspect, is a method of using DNA editing agent that contains a safe-lock element to generate a chimeric bird.

In another embodiment, there is provided a method of using DNA editing agent that contains a safe-lock element to induce lethality in a male embryo of a bird, the method comprises the steps of: administering such DNA editing agent to a population of bird cells, thereby generating genome-edited bird cells; transferring these genome-edited bird cells to recipient bird embryos; and exposing the embryos to an agent that removes the STOP element from the DNA editing agent, thereby eliciting expression of the lethality-promoting protein encoded by the DNA editing agent and inducing lethality in male embryos of the bird.

According to an aspect of some embodiments of the present invention there is provided a DNA editing agent comprising a first nucleic acid sequence for eliciting in an inducible manner a lethal phenotype of male chick embryos in an egg of a bird and a second nucleic acid sequence for directing the first nucleic acid sequence for effecting the lethal phenotype to a Z chromosome of a cell of the bird.

According to an aspect of some embodiments of the present invention there is provided a cell population comprising cells of a bird which comprise an exogenous polynucleotide which is stably integrated into the Z chromosome of the cells, the exogenous polynucleotide being for eliciting a lethal phenotype in a male offspring of the bird.

According to an aspect of some embodiments of the present invention there is provided a chimeric bird generated according to the method described herein.

According to an aspect of some embodiments of the present invention there is provided a transgenic bird generated using a gamete of the chimeric bird described herein.

According to an aspect of some embodiments of the present invention there is provided a method of reducing the number of male chicks hatching from the fertilized eggs of a bird, wherein an exogenous polynucleotide is stably integrated into the Z chromosome of the bird, the exogenous polynucleotide being for eliciting in an inducible manner a lethal phenotype in a male offspring of the bird, the method comprising exposing the eggs to an inducer which elicits the lethal phenotype, thereby reducing the number of male chicks hatching from the fertilized eggs of a bird.

According to an aspect of some embodiments of the present invention there is provided a chimeric bird comprising the cell population described herein.

According to an aspect of some embodiments of the present invention there is provided a method of generating a chimeric bird comprising administering the cell population described herein to a recipient bird embryo under conditions sufficient to allow at least one of the primordial germ cells (PGCs) of the cell population to colonize a gonad of the recipient bird embryo, thereby generating a chimeric bird.

According to an aspect of some embodiments of the present invention there is provided a DNA editing system comprising:
(i) a first agent comprising a first nucleic acid sequence for eliciting a lethal phenotype in an egg of a bird operatively linked to a recombinase recognition site and a sequence for directing said first nucleic acid sequence for effecting said lethal phenotype to a Z chromosome of a cell of the bird; and
(ii) a second agent comprising a second nucleic acid sequence which encodes a recombinase enzyme and a sequence for directing said second nucleic acid sequence to a Z chromosome of a cell of the bird.

According to an aspect of some embodiments of the present invention there is provided a method of reducing the number of male chicks hatching from the fertilized eggs of a bird comprising:
mating a female bird with a male bird, wherein a first exogenous polynucleotide which is operatively linked to a recombinase recognition site is stably integrated into the Z chromosome of said male bird, said exogenous polynucleotide being for eliciting a lethal phenotype in an egg of a bird and a second exogenous polynucleotide encoding a recombinase enzyme is stably integrated into the Z chromosome of said female bird, or
said first exogenous polynucleotide which is operatively linked to a recombinase recognition site is stably integrated into the Z chromosome of said female bird, said exogenous polynucleotide being for eliciting a lethal phenotype in an egg of a bird and a second exogenous polynucleotide encoding a recombinase enzyme is stably integrated into the Z chromosome of said male bird,
thereby reducing the number of male chicks hatching from the fertilized eggs of a bird.

According to some embodiments of the invention, the eliciting is effected in an inducible manner.

According to some embodiments of the invention, the first nucleic acid sequence encodes a lethality protein which is operatively linked to a nucleotide sequence encoding a switch that controls the expression of the lethality protein, the switch being regulated by an inducer.

According to some embodiments of the invention, the DNA editing agent is a single molecule.

According to some embodiments of the invention, the first nucleic acid sequence and the second nucleic acid sequence are comprised in non-identical molecules.

According to some embodiments of the invention, the first nucleic acid sequence encodes an endonuclease enzyme that can carry out genome editing which is operatively linked to a nucleotide sequence encoding a switch that controls the expression of the endonuclease protein, the switch being regulated by an inducer.

Examples of endonuclease proteins include Zinc finger nucleases (ZFNs), transcription-activator like effector nucleases (TALENs) and CRISPR/Cas system, each of which being further described herein above.

According to some embodiments of the invention, the second nucleic acid sequence comprises:
(i) a left homology arm (LHA) nucleotide sequence that is substantially homologous to the 5′ region flanking a target gene locus in the Z chromosome of the bird; and
(ii) a right homology arm (RHA) nucleotide sequence that is substantially homologous to the 3′ region flanking the target gene locus in the Z chromosome of the bird.

According to some embodiments of the invention, the inducer is selected from the group consisting of heat, ultrasound, electromagnetic energy and a chemical.

According to some embodiments of the invention, the switch comprises a split recombinase enzyme that combines to form an active enzyme in the presence of the inducer.

According to some embodiments of the invention, the switch comprises an inducible promoter.

According to some embodiments of the invention, the endonuclease enzyme is an RNA-guided DNA endonuclease enzyme.

According to some embodiments of the invention, the DNA editing agent further comprises a nucleotide sequence that encodes for a guide RNA that targets an essential gene of the bird, the nucleotide sequence being operatively linked to a nucleotide sequence encoding the switch.

According to some embodiments of the invention, the essential gene is selected from the group consisting of BMPR1A, BMP2, BMP4 and FGFR1.

According to some embodiments of the invention, the DNA editing agent further comprises a nucleotide sequence that encodes for a reporter polypeptide.

According to some embodiments of the invention, the inducer is electromagnetic energy.

According to some embodiments of the invention, the cells comprise primordial germ cells (PGCs).

According to some embodiments of the invention, the cells comprise gametes.

According to some embodiments of the invention, the method further comprises incubating the chimeric bird to hatch following the administering.

According to some embodiments of the invention, the method further comprises raising the chimeric bird to sexual maturity, wherein the chimeric bird produces gametes derived from the donor PGCs.

According to some embodiments of the invention, the administering is by in-ovo injection.

According to some embodiments of the invention, the cell population are derived from the same avian species as the recipient bird.

According to some embodiments of the invention, the cell population are derived from a different avian species as the recipient bird.

According to some embodiments of the invention, the first nucleic acid sequence encodes a lethality protein or an endonuclease enzyme that can carry out genome editing.

According to some embodiments of the invention, the sequence for directing said first or said second nucleic acid sequence to the X chromosome comprises:
(i) a left homology arm (LHA) nucleotide sequence that is substantially homologous to the 5′ region flanking a target gene locus in the Z chromosome of the bird; and
(ii) a right homology arm (RHA) nucleotide sequence that is substantially homologous to the 3′ region flanking said target gene locus in the Z chromosome of the bird.

Unless otherwise defined, all technical and/or scientific terms used herein have the same meaning as commonly understood by one of ordinary skill in the art to which an aspect or an embodiment pertains. Although methods and materials similar or equivalent to those described herein can be used in the practice or testing of embodiments of an aspect, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments are herein described, by way of example only, with reference to the accompanying drawings. With specific reference now to the drawings in detail, it is stressed that the particulars shown are by way of example and for purposes of illustrative discussion of certain embodiments. In this regard, the description taken with the drawings makes apparent to those skilled in the art how embodiments may be practiced.

The subject matter regarded provided herein is particularly pointed out and distinctly claimed in the concluding portion of the specification. The embodiments, however, both as to organization and method of operation, together with objects, features, and advantages thereof, may best be understood by reference to the following detailed description when read with the accompanying drawings in which:

FIG. 1 is a cartoon illustrating an embodiment of the generation of an optogenetic inducible chicken line from which only female layer chicks will hatch. By crossing wild-type rooster (ZZ) with genetically modified hen (ZW) all the female fertile eggs will carry wild-type ZW chromosomes. All the male fertile eggs will carry the ZZ chromosomes in which the genetically modified Z is derived from the genetically modified hen's genome. Upon blue light illumination of fertile eggs, the optogenetic system on this genetically modified chromosome will become active and will activate a death mechanism that will result in early male embryonic mortality soon after oviposition. The females which will not be affected by the blue light illumination will hatch, grow to adulthood and will lay unfertile eggs for food.

FIG. 2 illustrates an embodiment of a strategy to control gene expression by means of blue light illumination. Two fusion proteins are created: Cry2 with the non-active N-terminus of Cre (Cry2-CreN-term) and CIBN fused with the non-active C-terminus of Cre (CIBN-Cre-C-term). Without blue light illumination the Cre is inactive. Upon blue light illumination, Cry2 and CIBN form a complex and the two parts of Cre are brought together to form an active Cre enzyme.

FIG. 3 illustrates an embodiment of the homology arms on chromosome Z. The genomic region downstream to the HINT1Z locus is depicted. The 5′ and 3′ arms are HA-1 (Left homology arm; LHA) and HA-2 (Right homology arm; RHA), respectively. The primers for amplifying the arms are indicated by hollow arrows (FWD HA5′ P1 & REV HA3′ P2). In between the homology arms, on both DNA strands, there are sequences for CRISPR-Cas9 (open boxes, CRISPR Guide 1 & 3). Lower part of FIG. 3 shows in high detail the region between the two homology arms. The sequence set forth in SEQ ID NO: 1 is illustrated, including the LHA-CRISPR-RHA fragments.

FIGS. 4A-4C illustrate different embodiments of targeting vectors or DNA editing agents. FIG. 4A shows a targeting vector containing 3 main elements. The first, the 5′ and 3′ homology arms (HA) for homologous recombination (HR), flanking the entire exogenous insertion cassette. The second element is a light-inducible system—in this case the Cry2-CreN and the CIBN-Cre-C. The third element is the lethality gene cassette. In one embodiment of a single targeting vector strategy, the 5′ HA is followed by a pGK promoter that drives the expression of the Cry2-CreN and the CIBN-Cre-C genes, which are separated by a self-cleaving peptide P2A. This element is followed by the exogenous lethality gene cassette which contains a pGK promoter followed by a LoxP-STOP-LoxP site (LSL), which is followed by a lethality-inducing gene. This exogenous cassette is followed by the 3′ HA. Upon light induction, the Cry2-CreN and the CIBN-Cre-C dimerize to form an active form of Cre. The latter then excises the LSL element, thus allowing expression of the lethality-inducing gene, which leads to embryonic death in all embryos that carry this vector. FIG. 4B shows an alternative approach. Instead of using the LSL element, a Dio-lox flipping strategy is used. In between the Dio-Lox sites a GFP is followed by polyadenylation site #1 (PA1), and a lethality gene is followed by a different polyadenylation site #2 (PA2) in a reverse orientation. In this case, prior to light activation, the pGK promoter drives the expression of the GFP. Upon light activation, the cassette between the Dio-Lox sites flips and the lethality gene is now in the right orientation to be expressed while the GFP is now place in a reverse orientation and it is no longer active. FIG. 4C shows yet another embodiment. Following the activation of Cre, the LSL is removed and the Cas9 and single guide RNA (sgRNA) are expressed. This leads to the introduction of a missense mutation in the coding region of an essential gene (targeted by the sgRNA), thus inducing embryonic lethality.

FIGS. 5A-5F: PGCs line derivation and characterization. FIG. 5A, PGCs culture; FIG. 5B, left, mRNA expression of different pluripotent and germ-cells markers as indicated. Right, representative characterization of sex identification of female PGCs (left, two PCR products of Ribosomal S18 and W chromosome) and male PGCs (right, Ribosomal S18 only). FIG. 5C, PGCs staining with anti-SSEA1 antibodies. FIG. 5D, transfection of PGCs with pCAGG-GFP plasmid using Lipofectamine 2000 reagent. FIG. 5E, transfection of PGCs with pCAGG-GFP plasmid using electroporation. FIG. 5F, Gonad (testis) of an embryo, 10 days following transplantation with GFP-expressing cultured PGCs.

FIGS. 6A-6C. Designing the sgRNA sites for CRISPR-mediated targeting. FIG. 6A shows an example of the genomic area on the Z chromosome for potential CRISPR targeting sites. FIG. 6B shows 12 top sgRNA sequences (Guide #1-#12). Guides #1 and #3, which partially overlap, in opposite orientations, were chosen for further experiments. The 3-nucleotide PAM sequences are not part of the guide sequences, and the PAM sequences are not included in the SEQ ID NOs:66-77. The top 10 results of search for potential off-targets for guide #1 are shown in FIG. 6C and SEQ ID NOs:78-87. The depicted sequences do not include the 3-nucleotide PAM sequences.

FIGS. 7A-7C. Validating CRISPR activity using endonuclease assay. FIG. 7A. Positive control of the endonuclease assay using annealed WT 320 bp PCR product and a mutated product at the predicted cleavage site of CRISPR1 at the indicated ratios. FIG. 7B. Endonulease assay on 12 colonies transfected with CRISPR1 plasmid. FIG. 7C. Endonulease assay on 12 colonies transfected with CRISPR3 plasmid. There is a 12 bp distance between the two predicted cleavage sites of CRISPR1 and CRISPR3.

FIGS. 8A-8D. Validating CRISPR activity using DNA Sequencing. FIG. 8A. DNA chromatogram of wild type (WT) genomic region at the predicted cleavage site of CRISPR1, showing a normal sequence as a negative control. FIG. 8B. Sequence of mixture of WT and artificially mutated PCR products showing the appearance of double-peaks (arrowhead) after the predicted cleavage site, as a positive control. FIG. 8C. Sequencing of negative colony showing a normal sequence. FIG. 8D. Sequence of positive colony, showing the appearance of double peaks following the CRISPR1 cleavage site (arrowhead).

FIGS. 9A-9F. Constructing targeting vector for genome integration into the Z chromosome. FIG. 9A—Genomic DNA was used as a template for PCR reaction with primers P1 and P2 which are in the 5′HA and 3′HA regions (demarcated by dashed line), flanking the CRISPR-site-containing region. FIG. 9B—The ~3 kb product, located downstream to the HINT1Z locus was ligated to the shuttle vector pJet1.2. This plasmid was used as a template for PCR with primers P3 and P4. These primers have extension overhang sequences (demarcated by curly brackets) which correspond to the equivalent regions on the pCAGG-Neo-IRES-GFP fragment. FIG. 9C—The linearized product (the vector) containing the two homology arms, excluding the CRISPR-site-containing region, flanked by sequences which bind the ends of the pCAGG-Neo-IRES-GFP cassette during the Gibson reaction. FIG. 9D—The pCAGG-Neo-IRES-GFP plasmid was used as template for PCR reaction with primers P5 and P6. These primers contain extension overhang sequences (demarcated by curly brackets) which correspond to the equivalent regions on the edges of the homology arms. FIG. 9E—The linearized insert cassette flanked by sequences which bind the ends of the homology arms. The vector and the insert were stitched together by the Gibson assembly reaction to create the final targeting vector plasmid as shown in FIG. 9F.

FIGS. 10A-10D. Co-transfection of targeting vector and CRISPR plasmids to PGCs. FIG. 10A. Lipofection-mediated co-transfection to PGCs with CRISPR1 and HR targeting vector plasmids. FIG. 10B. Two weeks after G-418 selection, >99% of the resistant PGCs were positive for GFP. FIG. 10C. Ten days following the injection of targeted PGCs to a host embryo, numerous cells were found to be localized in the gonads (testis). FIG. 10D. The gonads were dissected, immuno-stained with anti-GFP antibody, and scanned using confocal microscope (GFP antibody staining in green and nuclei counterstained with 4′, 6 diamidino-2-phenylindole (DAPI) in blue in original).

FIGS. 11A-11D. Verification of HR integration in FACS sorted PGCs. FIG. 11A. FACS sorting of G-418 resistant PGCs. FACS gating was designed to sort singular (sin) GFP-positive cells that were sorted as pool or individual cells in 96 well plate. FIG. 11B. For PCR analysis two sets of primers for the 5′ integration site (P7 and P8) and the 3′ integration site (P9 and P10), were designed. FIG. 11C. Genomic DNA extracted from the pooled cells was used as a template for the PCR and WT DNA served as a negative control. The predicted 1.6 kb and 1.8 kb bands were evident for the correct HR integration in the 5′ and 3′ regions, respectively. FIG. 11D. Genomic DNA extracted from male and female cell colonies, originated from single cell FACS sorted PGCs, was used as a template for the PCR and WT DNA served as a negative control. The predicted 1.6 kb and 1.8 kb bands were evident for the correct HR integration in the 5′ and 3′ regions, respectively.

FIGS. 12A-12D. Southern blot analysis of the HR integration. FIG. 12A—schematic representation of the expected BglII cleaved products in Southern blot analysis for the WT allele and the allele which underwent HR integration. The probes used for 5′, 3′ integration sites and for the neo are marked as short bars. The expected product size, following BglII digestion, for each DNA probe are shown. FIG. 12B—Preparation of the Dig-labeled probes by PCR. Dig-labeled probes (+) or un-labeled (−) were analyzed on an agarose gel. Note that Dig-labeled products are shifted higher than their actual size, confirming the integration of the Dig-labeled nucleotides. The sets of primers used for amplifying the probes are indicated. FIG. 12C—Southern blot analysis with the 5′ and 3′ probes on DNA extracted from pooled and pure colonies of male-derived PGCs. WT DNA extracted from the original line, prior to the HR, served as a negative control. FIG. 12D—Southern blot analysis with the 5′ and the Neo probes on female-derived PGCs. A single 7.5 kb band is evident in both cased, indicating that correct HR occurred and only a single copy of the targeting vector was integrated.

FIG. 13. Validation of the optogenetic system in HEK293 cells, in-vitro. Triple transfection with pmCherry-Cry2-CreN, pmCherry-CIBN-Cre-C and PB-RAGE-GFP plasmids. Twenty-four hours following transfection, cells in experimental group were exposed for 15 seconds of blue light illumination while control cells were kept in dark (upper row). Following illumination (lower row), the cells were further incubated for 24 hours. In these cells, GFP expression was evident confirming the activation of the Cre enzyme upon blue-light illumination.

FIG. 14. Validation of the optogenetic system in-ovo in chick embryos, incubated for 54-60 hours prior to electroporation. Triple electroporation to chicken embryos with pmCherry-Cry2-CreN, pmCherry-CIBN-CreC and pB-RAGE-GFP plasmids. Twelve hours following electroporation, experimental group embryos were exposed for 15 seconds of blue light illumination in-ovo while control embryos were kept in dark (upper row). Following illumination (lower row), the embryos from both groups were incubated for additional 12 hours. Following incubation, GFP expressing cells were clearly evident in the illuminated group, thus confirming the activation of the optogenetic system and the Cre enzyme upon blue-light illumination in chicken embryos in-ovo.

FIGS. 15A-15F. Constructing a single optogene expression vector under the CAGG promoter. FIG. 15A. The optogenes plasmids pmCherry-CIBN-CreC and pmCherry-Cry2-CreN were used as a template to amplify the optogenes fusion proteins using the P40-P41 and P42-P43 primers, respectively. The two products share overlap sequences at the P2A site which was introduced in primers P41 and P42. This allowed for single-cycle overhang extension PCR to unite the two fragments (see FIG. 15B) into one piece, which was ligated to pJet1.2 shuttle vector as shown in FIG. 15C. Using primers P44 and P45, which contain tails with SmaI and NheI restriction sites, respectively, the product in FIG. 15D was generated. This product was digested using the appropriate restriction enzymes and was ligated to pCAGG-IRES-GFP (FIG. 15E) that was digested with the same enzymes to obtain the vector as shown in FIG. 15F.

FIG. 16. Validating the activity of the pCAGG-Optogene plasmid in HEK293 cells. Co-transfection with pCAGG-Optogene and pB-RAGE-mCherry plasmids. Twenty-four hours following transfection, while the negative-control group was kept in the dark (upper row), the experimental group cells were exposed for 15 seconds to blue light illumination (lower row). Following illumination (lower row), the cells were further incubated for 24 hours. In these cells, mCherry expression was evident (white arrows) confirming the activation of the Cre enzyme by the pCAGG-Optogene plasmid, upon blue-light illumination.

FIG. 17. Verification of the single-vector strategy using the pCAGG-Optogenes plasmid in-ovo. Chicken embryos at stage 14-16H&H were co-electroporated with pCAGG-Optogenes and pB-RAGE-mCherry plasmids. The latter plasmid serves as a reporter gene for the activity of the optogenetic system. Twelve hours following electroporation, the experimental group embryos (lower row) were exposed for 15 sec to blue light illumination in-ovo while control embryos were kept in the dark (upper row). The embryos were further incubated for 12 hours. Following incubation, GFP expressing cells were clearly evident in both groups, indicating successful electroporation, however, only in the illuminated group mCherry-expressing cells were evident, confirming the activation of the optogenetic system and the Cre enzyme upon blue-light illumination in chicken embryos in-ovo.

FIG. 18. Expression of DTA under the pGK promoter inhibits protein synthesis in-ovo. Stage 14-16 H&H embryos were electroporated with either the pGK-IRES-GFP (upper row) or pGK-DTA-IRES-GFP (lower row) expression vector. Negative control embryos widely express GFP (upper row, arrow) indicating normal protein synthesis. DTA expressing cells show no GFP expression (lower row), indicating that protein synthesis in these embryos is inhibited. GFP-only, bright-field-only and GFP overlaid on bright-field images are presented.

FIGS. 19A-19B. Embodiments of targeting vectors. In these vectors the activating enzyme (Cre for example) is separated from the lethality gene cassette. In FIG. 19A, the activating enzyme is inserted into the genome of the mother hen and the inactive lethality cassette is inserted on the Z chromosome of the rooster, which is homozygote to this allele. In this case the activation of lethality in male embryos is carried out by crossing the two transgenic parents without the need for light induction. The Cre in all males removes the LSL on the maternal derived Z chromosome thereby allowing the lethality gene to be expressed, while the female embryo harbors an inactive lethality cassette, thus it is unaffected. FIG. 19B. Alternatively, the Z chromosome on the mother hen is targeted with Dio-Lox flipping cassette containing the FLP recombinase in the right direction followed by a lethality gene in reverse orientation, driven by the CAGG promoter. The rooster, again homozygote to the Z chromosome which is targeted with CAGG-Cre cassette flanked by FRT sites. Upon crossing the two, male embryos will express the Cre located on the paternal Z chromosome, the Dio-Lox cassette flips and the lethality gene becomes active, thereby leading to embryonic lethality of the male embryo. The zygote of the female embryo from this cross contains maternal contribution of the FLP recombinase enzyme that was produced during oogenesis. This maternal protein, removes the CAGG-Cre cassette from the Z chromosome, leaving the female embryo alive with only a FRT “scar” on the Z chromosome.

FIG. 20. RT-PCR on cDNA from total RNA extract from whole freshly-laid blastoderms (Bl) and PGCs, with primers for HINT1Z and GAPDH as a positive control (GAPDH primers: Forward—(SEQ ID NO: 90); Reverse—(SEQ ID NO: 91), 93 bp). Bands at the predicted size of 153 bp indicate that in both samples, HINT1Z which is located on the Z chromosome is transcribed.

FIG. 21. Schematic diagram of two plasmids containing the modified optogenetic system (pCAGG-CIBN-Cre-C-P2A-Cry2-Cre-N and pCAGG-Cry2-Cre-N-P2A-CIBN-Cre-C).

FIG. 22. Validation of the optogenetic system in cultured HEK293 cells. The optogenetic plasmid pCAGG-CIBN-CreC-IRES-Cry2-CreN-IRES-GFP was co-transfected with pB-RAGE-mCherry. Like the PB-RAGE-GFP vector described above, the pB-RAGE-mCherry contains a multiple stop codon sequence flanked by LoxP sites upstream to the mCherry coding region. Upon Cre activation, the stop codons are removed thus allowing the mCherry to be expressed. While in HEK293 cells that were co-transfected and kept in the dark there were no mCherry-positive cells, in the co-transfected HEK293 cells that were exposed to blue-light illumination, many cells were expressing mCherry, confirming that the single-vector strategy of the pCAGG-Optogenes preserves the optogenetic properties of the system.

FIG. 23. Validation of optogenetic system by electroporation in chicken embryos. pCAGG-CIBN-IRES-Cry2, which also encodes GFP, was electroporated along with PB-RAGE-mCherry, which gives red fluorescence when in the presence of an active Cre recombinase. When induced with blue light, Cry2-CreN and CIBN-CreC dimerize, thus enabling Cre activity. White circles indicate areas of overlapping fluorescence.

FIGS. 24A-24B. Induction of cell death in PGCs using DTA (FIG. 24A) or Caspase3 (FIG. 24B). FIG. 24A. The effect of DTA on PGC's cell death is presented. PGCs were transfected with control 1 PGK-IRES-GFP, control 2 pCAGG-GFP, or PGK-DTA-IRES-GFP with pCAGG-GFP plasmids and incubated for 24, 48, and 72 h. Cell death was assessed using flow-cytometry for GFP and PI. Results present the ratio between GFP+PI and GFP only cells. FIG. 24B. The effect of Casp's on PGC's cell death is presented. PGCs were transfected with control PGK-IRES-GFP, PGK-WT Caspase3-IRES-GFP or PGK-CA Caspase3-IRES-GFP plasmids and incubated for 24, 48, and 72 h before analysis as illustrated in FIG. 24A.

FIG. 25. Schematic illustration of the targeting vector, containing all elements and the activation of the lethality-inducing cassette upon blue-light illumination.

FIGS. 26A-26B. Schematic illustration of the targeting vectors containing a “safe-lock” mechanism as explained in Example 3.

FIGS. 27A-27D. Validation of targeting vectors in cultured HEK293 cells. For in-vitro validation, HEK293 cells were transfected with TV4 alone (FIG. 27A), with pCAGG-Cre (FIG. 27B), or with pCAGG-FlpO plasmids (FIGS. 27C-27D). The cells were kept in dark (FIG. 27C) or was exposed for 15 seconds to blue light 24 hours following transfection (FIG. 27D). Following illumination, the cells were further incubated for 24 hours.

FIGS. 28A-28D. Validation of targeting vectors by electroporation in chicken embryos. Chicken embryos were injected with plasmids to the neural tube and electroporated as described herein. The white lines denote the dorsal mid-line of the neural tube and limb buds for orientation purposes. Four treatment groups were tested: 1. expression of TV4 alone (FIG. 28A), 2. co-electroporation of TV4 and pCAGG-Cre plasmids, as positive control (FIG. 28B), 3. Co-electroporation of TV4 and pCAGG-FlpO plasmids. The cells were kept in the dark (FIG. 28C), and 4. Exposure to blue light for 15 sec after co-electroporation of TV4 and pCAGG-FlpO plasmids, and further incubated for 12 hours (FIG. 28D).

FIGS. 29A-29B. The light-dependent activity of the lethality-inducing gene Noggin. Chick embryos were electroporated in the neural tube with targeting vector TV1, pCAGG-FlpO and pCAGG-IRES-GFP plasmids. Targeting vector TV1 contains the coding sequence ofNoggin as a lethality-inducing element. FIG. 29A shows results without light induction; upper row, dorsal view; lower row, right-lateral view. FIG. 29B shows results with blue light induction.

FIGS. 30A-30E. Noggin is able to stop embryonic development at the blastoderm embryonic stage. Blastoderms were treated with exogenous source of Noggin. Plasmids pCAGG-Noggin-IRES-GFP or pCAGG-IRES-GFP (as negative control) were transfected to HEK293 cells. FIG. 30A shows total protein extracted from transfected cells was analyzed by Western blot with anti-Noggin antibody and anti-α-Tubulin-HRP antibody. Conditioned media from control and Noggin expressing cells were injected into freshly-laid fertile eggs that were subsequently incubated for 24 hours (FIG. 30B-C) or 54 hours (FIG. 30D-E).

FIGS. 31A-31C. PGCs that underwent HR on the Z chromosome with TV1 successfully colonize the gonads in chick embryos. FIG. 31A shows pure female PGCs line that underwent HR with TV1, and expressed GFP. FIG. 31B shows a ventral view of an embryo 5 days following PGCs injection. The PGCs colonized the genital ridge which is the anlage of the gonads (FIG. 31B, arrowheads). Female chicks were sacrificed at day 10 post hatch to analyze the ovary. FIG. 31C shows an ovary (delineated by a line) containing numerous GFP positive PGCs.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the compositions and methods provided herein. However, it will be understood by those skilled in the art that the present disclosure may be practiced without these specific details. In other instances, well-known methods, procedures, and components have not been described in detail so as not to obscure the compositions and methods provided herein.

Before explaining at least one embodiment of the disclosure in detail, it is to be understood that the disclosure is not necessarily limited in its application to the details set forth in the following description or exemplified by the Examples. The disclosure encompasses other embodiments or can be practiced or carried out in various ways.

The present invention, in some embodiments thereof, relates to genome edited birds which lay eggs comprising non-viable male chick embryos.

The terms “comprises”, “comprising”, “includes”, “including”, “having” and their conjugates encompass “including but not limited to”.

As used herein, the singular form “a”, “an” and “the” include plural references unless the context clearly dictates otherwise. For example, the term “a compound” or “at least one compound” may include a plurality of compounds, including mixtures thereof.

In some embodiments, the term “about” refers to a deviance of between 0.0001-5% from the indicated number or range of numbers. In some embodiments, the term “about” refers to a deviance of between 1-10% from the indicated number or range of numbers. In some embodiments, the term “about” refers to a deviance of up to 25% from the indicated number or range of numbers. In some embodiments, the term “about” refers to +10%.

Throughout this application, various embodiments may be presented in a range format. It should be understood that the description in range format is merely for convenience and brevity and should not be construed as an inflexible limitation on the scope of certain embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

In one embodiment, the technology, products and methods provided herein generate a chicken breed in which only female layers will hatch while male embryos will cease to develop soon after fertilization. Thus, the need to cull the male chicks is eliminated and 50% of valuable incubation space is saved in hatcheries. Importantly, both the females and the eggs derived from the methods disclose herein are in every aspect identical to the layer-hens and food-eggs currently consumed by the public.

As used herein, the terms “bird” and “avian species” refer to any avian species, including but not limited to chicken, turkey, duck, geese, quail, pheasant, and ostrich.

In certain embodiments, the bird is a female. In certain embodiments, the bird is a male. In certain embodiments, the bird is a broiler. In certain embodiments, the bird is a hen. In certain embodiments, the bird is layer hen. In certain embodiments, the bird is a domestic chicken. In certain embodiments, the bird is Gallus gallus domesticus layer hen.

As used herein, the term “egg” refers to an avian egg that contains a live embryonic bird. Thus, the term “egg” is intended to refer to a fertilized avian egg, in one embodiment an egg containing an avian embryo that is capable of undergoing normal embryogenesis.

In one embodiment, provided herein is a DNA editing agent comprising a polynucleotide cassette having a formula 5′-LHA (left homology arm)-OIE (optogenetic-inducible element)-LIE (lethality-inducing element)-RHA (right homology arm)-3′ or a formula 5′-LHA-LIE-OIE-RHA-3′, wherein (i) the LHA comprises a first nucleotide sequence that is substantially homologous to a first corresponding nucleotide sequence on chromosome Z of a bird; (ii) the OIE comprises a first promoter functionally linked to a second nucleotide sequence encoding an inducer-activated site-specific recombinase enzyme; (iii) the LIE comprises a third nucleotide sequence encoding a lethality-promoting protein, which is operatively linked to the activity of the inducer-activated site-specific recombinase enzyme; and (iv) the RHA comprises a fourth nucleotide sequence that is substantially homologous to a second corresponding nucleotide sequence on chromosome Z of a bird.

In certain embodiments, one or both of the LHA and RHA are substantially homologous to a corresponding nucleotide sequence located in an openly transcribed region on chromosome Z of a bird. For example, the openly transcribed region can be located at or downstream to the histidine triad nucleotide binding protein 1-Z (HINT1Z) locus on chromosome Z of a bird.

In certain embodiments, the inducer-activated site-specific recombinase enzyme can be Cre recombinase (Cre) (SEQ ID NO: 113), or Mag (SEQ ID NO: 114 and SEQ ID NO: 65). In certain embodiments, the expression of the inducer-activated site-specific recombinase enzyme is induced by an inducer. In certain embodiments, the inducer is electromagnetic energy. For example, the inducer can be blue light having a wavelength of 450-485 nm.

In certain embodiments, the inducer-activated site-specific recombinase enzyme comprises non-functional peptide fragments of an inducer-activated site-specific recombinase that combine to form an active inducer-activated site-specific recombinase enzyme in the presence of the inducer.

In certain embodiments, the lethality-inducing protein mentioned herein can be a toxin, a pro-apoptotic protein, an inhibitor of the Wingless/Integrated (Wnt) signaling pathway, a bone morphogenetic protein (BMP) antagonist, a fibroblast growth factor (FGF) antagonist, a wild type Caspase 3, a constitutively active Caspase 3, Noggin, or a lethality-inducing fragment of any of the above proteins.

According to other embodiments, there is provided a DNA editing agent comprising a first nucleic acid sequence for eliciting in an inducible manner a lethal phenotype of male chick embryos in an egg of a bird and a second nucleic acid sequence for directing the first nucleic acid sequence for effecting the lethal phenotype to a Z chromosome of a cell of the bird.

According to other embodiments, there is provided a DNA editing system comprising:
(i) a first agent comprising a first nucleic acid sequence for eliciting a lethal phenotype in an egg of a bird operatively linked to a recombinase recognition site and a sequence for directing said first nucleic acid sequence for effecting said lethal phenotype to a Z chromosome of a cell of the bird; and
(ii) a second agent comprising a second nucleic acid sequence which encodes a recombinase enzyme and a sequence for directing said second nucleic acid sequence to a Z chromosome of a cell of the bird.

As mentioned, the DNA editing agent (or system) is used for the generation of female chickens that are only capable of producing viable female offspring and not viable male offspring.

As a first step, the DNA editing agent is introduced into either primordial germ cells of the bird or directly into sperm cells of the bird.

Approaches for introducing nucleic acids of interest into recipient cells are known and include lipofection, transfection, microinjection, electroporation, transformation and microprojectic techniques, etc.

Thus, according to another aspect of the present invention there is provided a cell population comprising cells (e.g. primordial germ cells or gametes) of a bird which comprise an exogenous polynucleotide which is stably integrated into the Z chromosome of the cells, said exogenous polynucleotide being for eliciting (e.g. in an inducible manner) a lethal phenotype in a male offspring of the bird (and not in the female offspring of the bird).

The DNA editing agent (comprised in a single nucleic acid construct or a combination of nucleic acid constructs) comprises targeting sequences which brings about stable integration of the first nucleic acid sequence into the Z chromosome of a cell of the bird. The DNA editing agent may be constructed using recombinant DNA technology well known to persons skilled in the art.

The targeting sequences (for example LHA and/or the RHA targeting sequences) are selected such that the first nucleic acid sequence integrates specifically into the Z chromosome and not any other chromosome of the cell. Furthermore, the targeting sequence is selected depending on what method is being relied upon to integrate the first nucleic acid sequence into the chromosome. Methods of integrating nucleic acid sequences into the chromosome are well known in the art [see for example Menke D. Genesis (2013) 51: —618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases and genome editing by engineered nucleases. PB transposases are also contemplated. Agents for introducing nucleic acid alterations to a gene of interest can be designed publically available sources or obtained commercially from Transposagen, Addgene and Sangamo Biosciences.

In one embodiment, the DNA editing agent relies on spontaneous homologous recombination to insert the first nucleic acid sequence into the Z chromosome of the cell. In this embodiment, the DNA editing agent comprises homology arms that serve as targeting sequences.

The DNA editing agent may be flanked with two arms that are homologous or show homology or identity of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% to at least one nucleic acid sequence comprised within the target loci within the Z chromosome, that serves as the integration site to facilitate specific integration via HDR.

The homology arms correspond to a genomic sequence which is present on the Z chromosome. Preferably, the genomic sequence is downstream of a gene which is transcriptionally active (for example downstream to the Hint1 gene). The homology arms are typically at least 500 nucleotides long, for example between 500-3000 nucleotides long. Typically, the required size of the homology arms relies on the length of the cassettes which are flanked by these arms. Smaller cassettes require shorter arms and vice versa. Homologous recombination can occur spontaneously. Another contemplated target is Isl1 (Gene ID 369383), also on chromosome Z which is expressed starting from early stages of embryogenesis.

Following is a description of various additional exemplary methods which may be used by the DNA editing agent to integrate the first nucleic acid sequence into the Z chromosome according to specific embodiments of the present invention and a description of the targeting sequences that are required to carry out this purpose.

### Genome Editing

This approach refers to a reverse genetics method using artificially engineered nucleases to cut and create specific double-stranded breaks at a desired location(s) in the genome (i.e. on the Z chromosome), which are then repaired by cellular endogenous processes such as, homology directed repair (HDR) and non-homologous end-joining (NIIEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8, 129,134; 8,133,697; 8,143,015; 8,143,016; 8, 148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically, a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally, Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the nonhomologous end-joining (NHEJ) pathway most often results in indels which are small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, CA).

CRISPR-Cas system—Many bacteria and archaea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcuspyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the CRISPR design tool from Feng Zhang lab's, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids. CRISPR plasmids are publicly available such as the px330 plasmid from Addgene. Additionally, mRNA encoding Cas9 and the gRNA can be inserted to the target cells as well as recombinant Cas9 protein in complex with the gRNA (i.e. insert the RNP complex into the cell).

In certain embodiments, the CRISPR/Cas system is a Class 1 CRISPR/Cas system. In certain embodiments, a Class 1 CRISPR/Cas system comprises a multi-subunit crRNA-effector complex. In certain embodiments, the CRISPR/Cas system is a type I CRISPR-Cas system. In certain embodiments, the CRISPR/Cas system is a type III CRISPR/Cas system. In certain embodiments, the CRISPR/Cas system is a type IV CRISPR-Cas system.

In certain embodiments, the CRISPR/Cas system is a Class 2 CRISPR/Cas system. In certain embodiments, a Class 2 CRISPR/Cas system comprises a single subunit crRNA-effector module. In certain embodiments, the CRISPR/Cas system is a type II CRISPR-Cas system. In certain embodiments, the CRISPR/Cas system is a type V CRISPR/Cas system.

In certain embodiments, the Cas in the Class 2 CRISPR/Cas system can be Cas9, Cpf1, C2c1, C2c2 or C2c3. A person of ordinary skill in the art would understand the classification of CRISPR/Cas systems as it is well-known in the art (e.g. Nat Rev Microbiol. 2017 March, 15(3): 169-182; Nat Rev Microbiol. 2015 November, 13(11): 722-736), and that this classification is evolving with time (Mol Cell. 2015 November 5, 60(3): 385-397). In some embodiments, the CRISPR/Cas is any CRISPR associated protein (CAS) endonuclease known in the art.

In order to facilitate homologous recombination, a chemical inhibitor of NHEJ such as SCR7 pyrazine may be used to enhance CRISPR genome editing-mediated HR efficiency. As mentioned, the DNA editing agent of this aspect of the present invention comprises a first nucleic acid sequence for eliciting in an inducible manner a lethal phenotype of male chicks hatching from an egg of a bird.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and Western blot analysis and immunohistochemistry.

The DNA editing agents and systems of the present invention may also comprise a reporter gene, which encodes a reporter polypeptide that is readily detectable either by its presence or activity, including, but not limited to, luciferase, fluorescent protein (e.g., green fluorescent protein), chloramphenicol acetyl transferase, beta-galactosidase, secreted placental alkaline phosphatase, beta-lactamase, human growth hormone, and other secreted enzyme reporters. Generally, a reporter gene encodes a polypeptide not otherwise produced by the host cell, which is detectable by analysis of the cell(s), e.g., by the direct fluorometric, radioisotopic or spectrophotometric analysis of the cell(s) and typically without the need to kill the cells for signal analysis. In certain instances, a reporter gene encodes an enzyme, which produces a change in fluorometric properties of the host cell, which is detectable by qualitative, quantitative, or semiquantitative function or transcriptional activation. Exemplary enzymes include esterases, beta-lactamase, phosphatases, peroxidases, proteases (tissue plasminogen activator or urokinase) and other enzymes whose function can be detected by appropriate chromogenic or fluorogenic substrates known to those skilled in the art or developed in the future. The reporter gene may report on successful integration of the construct into the Z chromosome.

Other components of the DNA editing agent include self-cleaving peptides such as the 2A, including but not limited to P2A, T2A, E2A (Wang et al., Scientific Report 5, Article 16273 (2015), or internal ribosome entry site (IRES) sequences.

In addition, one ordinarily skilled in the art can readily design a DNA editing agent which includes a positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT), Diphtheria toxin (DT) and adenine phosphoribosyltransferase (ARPT).

### DNA Editing Agent

The technology described herein provides, in certain aspects and embodiments, a DNA-editing agent. The DNA editing agent may be constructed using recombinant DNA technology well known to persons skilled in the art.

In one embodiment, the DNA editing agent disclosed herein may be comprised in a single nucleic acid construct, or comprised in a combination of nucleic acid constructs. In one embodiment, the DNA editing agent comprises at least two key elements as described below:

A first element is a nucleotide sequence cassette which is destined to be stably integrated to a specific location in the genome of a bird. The first element, when integrated into the genome of a bird, changes the genotype of the bird, but also, in certain embodiments and under certain conditions, changes the phenotype of the bird. The altered phenotype of the bird, in comparison to the phenotype of other birds, is the purpose of the technology provided herein. Briefly, and as described in detail by the embodiments provided herein, the altered phenotype is useful in the prevention of the development of viable male chicks out of male embryos. This prevention can save farmers and hatcheries significant economic burdens, as well as preventing the need to sacrifice viable male chicks.

A second element is a first and second nucleotide targeting sequences that flank the first element. The second element is in charge of determining the location in a genome of a bird into which the first element is stably integrated. Random integration of foreign DNA into a genome of any organism would be detrimental if it interferes with genes in charge of basic cell functions. Alternatively, random integration may be immaterial if the foreign DNA integrates to a non-active segment of the DNA. The second element performs the important function of directing integration of the first element into a defined and predetermined region in the bird's DNA. In one embodiment of the present disclosure, the second element directs the incorporation of the first element into openly transcribed regions in chromosome Z of birds, without having any negative effect on basic cell functions.

A person of skill in the art would understand that the term “DNA editing agent” generally refers to any molecule, such as a nucleotide sequence or an enzyme, which promotes a change in a genome of an organism, such as a bird. The change may be an addition to the DNA, e.g. by the agent being integrated to the DNA, a replacement of a sequence of the DNA, e.g. by homological recombination, or a deletion of the DNA.

The DNA editing agent may be constructed in a viral vector (using a single vector or multiple vectors). Such vectors are commonly used in gene transfer and gene therapy applications. Different viral vector systems have their own unique advantages and disadvantages. Viral vectors that may be used to integrate the first nucleic acid sequence of the present invention into the Z chromosome include but are not limited to adenoviral vectors, adeno-associated viral vectors, alphavirus vectors, herpes simplex viral vectors, and retroviral vectors, described in more detail below. According to a particular embodiment, the vector is a lentiviral vector.

A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

In one embodiment, provided herein is a DNA editing agent comprising a polynucleotide cassette having a formula 5′-LHA (left homology arm)-OIE (optogenetic-inducible element)-LIE (lethality-inducing element)-RHA (right homology arm)-3′ or a formula 5′-LHA-LIE-OIE-RHA-3′, wherein (i) the LHA comprises a first nucleotide sequence that is substantially homologous to a first corresponding nucleotide sequence on chromosome Z of a bird; (ii) the OIE comprises a first promoter functionally linked to a second nucleotide sequence encoding an inducer-activated site-specific recombinase enzyme; (iii) the LIE comprises a third nucleotide sequence encoding a lethality-promoting protein, which is operatively linked to the activity of the inducer-activated site-specific recombinase enzyme; and (iv) the RHA comprises a fourth nucleotide sequence that is substantially homologous to a second corresponding nucleotide sequence on chromosome Z of a bird.

As it would be apparent to those skilled in the art, a formula 5′-LHA-OIE-LIE-RHA-3′ or a formula 5′-LHA-LIE-OIE-RHA-3′ relate to the respective position of any element (e.g. LHA or OIE or LIE or RHA) in relation to all other elements in the same DNA editing agent molecule. The terminology of 5′ and 3′ is well accepted and is well-known to a person of ordinary skill in the art.

In certain embodiments, the polynucleotide cassette comprises the formula 5′-LHIA-OIE-LIE-RHA-3′. In certain embodiments, the OIE is upstream to the LIE. In certain embodiments, the first promoter in the OIE is functionally linked to the second nucleotide sequence encoding the inducer-activated site-specific recombinase enzyme and is not further functionally linked to the third nucleotide sequence encoding the lethality-promoting protein.

In certain embodiments, the polynucleotide cassette comprises the formula 5′-LHA-LIE-OIE-RHA-3′. In certain embodiments, the LIE is upstream to the OIE. In certain embodiments, the first promoter in the OIE is functionally linked to the second nucleotide sequence encoding the inducer-activated site-specific recombinase enzyme and is not further functionally linked to the third nucleotide sequence encoding the lethality-promoting protein.

In certain embodiments, the DNA editing agent comprises a formula 5′-LHA-OIE-LIE-RHA-3′, wherein (i) LHA comprises the sequence set forth in SEQ ID NO: 105, (ii) OIE comprises the sequences set forth in SEQ ID NOs: 101-103, or the sequences set forth in SEQ ID NO: 107, SEQ ID NO: 103, and SEQ ID NO: 108, (iii) LIE comprises the sequence set forth in SEQ ID NO: 92 or comprises the sequence set forth in SEQ ID NO: 94 or the sequence set forth in SEQ ID NO: 96 or the sequence set forth in SEQ ID NO: 98, (iv) RHA comprises the sequence set forth in SEQ ID NO: 106, or (v) any combination of (i), (ii), (iii) and (iv).

In certain embodiments, the OIE comprises the sequence set forth in SEQ ID NO: 101, which is connected to the sequence set forth in SEQ ID NO: 103, which is connected to the sequence set forth in SEQ ID NO: 102. In certain embodiments, the OIE comprises the sequence set forth in SEQ ID NO: 102, which is connected to the sequence set forth in SEQ ID NO: 103, which is connected to the sequence set forth in SEQ ID NO: 101.

In certain embodiments, the OIE comprises the sequence set forth in SEQ ID NO: 107, which is connected to the sequence set forth in SEQ ID NO: 103, which is connected to the sequence set forth in SEQ ID NO: 108. In certain embodiments, the OIE comprises the sequence set forth in SEQ ID NO: 108, which is connected to the sequence set forth in SEQ ID NO: 103, which is connected to the sequence set forth in SEQ ID NO: 107.

In certain embodiments, (i) the LHA comprises the sequence set forth in SEQ ID NO: 105, (ii) the OIE comprises the sequence set forth in SEQ ID NO: 100, which is connected to the sequence set forth in SEQ ID NO: 116, which is connected to the sequence set forth in SEQ ID NO: 101, which is connected to the sequence set forth in SEQ ID NO: 103, which is connected to the sequence set forth in SEQ ID NO: 102, which is connected to the sequence set forth in SEQ ID NO: 104, which is connected to the sequence set forth in SEQ ID NO: 116, or the OIE comprises the sequence of SEQ ID NO:100, which is connected to the sequence of SEQ ID NO:116, which is connected to the sequence of SEQ ID NO:102, which is connected to the sequence of SEQ ID NO:103, which is connected to the sequence of SEQ ID NO:101, which is connected to the sequence of SEQ ID NO:104, which is connected to the sequence of SEQ ID NO:116, or the OIE comprises the sequence set forth in SEQ ID NO: 100, which is connected to the sequence set forth in SEQ ID NO: 116, which is connected to the sequence set forth in SEQ ID NO: 107, which is connected to the sequence set forth in SEQ ID NO: 103, which is connected to the sequence set forth in SEQ ID NO: 108, which is connected to the sequence set forth in SEQ ID NO: 104, which is connected to the sequence set forth in SEQ ID NO: 116, or the OIE comprises the sequence of SEQ ID NO:100, which is connected to the sequence of SEQ ID NO:116, which is connected to the sequence of SEQ ID NO:108, which is connected to the sequence of SEQ ID NO:103, which is connected to the sequence of SEQ ID NO:107, which is connected to the sequence of SEQ ID NO:104, which is connected to the sequence of SEQ ID NO:116, (iii) the LIE comprises the sequence set forth in SEQ ID NO: 92 or comprises the sequence set forth in SEQ ID NO: 94 or the sequence set forth in SEQ ID NO: 96 or the sequence set forth in SEQ ID NO: 98, connected to the sequence set forth in SEQ ID NO: 104, and (iv) RHA comprises the sequence set forth in SEQ ID NO: 106.

In certain embodiments, the DNA editing agent comprises the sequence of one of SEQ ID NOs:120-127.

In certain embodiments, the DNA editing agent may comprise nucleotide sequence that encodes a reporter polypeptide. In certain embodiments, the reporter polypeptide can be a green fluorescence protein (GFP) (SEQ ID NO: 115), or mCherry/RFP (SEQ ID NO: 119).

In certain embodiments, the codons encoding the proteins of the DNA editing agent are “optimized” codons, i.e., the codons are those that appear frequently in, e.g., highly expressed genes in the bird species, instead of those codons that are frequently used by, for example, an influenza virus. Such codon usage provides for efficient expression of the protein in avian cells. Codon usage patterns are known in the literature for highly expressed genes of many species (e.g., Nakamura et al., 1996, Nucleic Acids Res. 24(1):214-5; McEwan et al., 1998, Biotechniques. 24(1):131-6, 138).

### Left and Right Homology Arms

It is generally accepted that the size of the homology arms for HR should be proportionate to the size of the insert between the arms. One of ordinary skill in the art would readily determine and construct a homology arm with suitable length. In one embodiment, the homology arm can be as short as 50 bases. In certain embodiments, (i) the length of the LHA is about 0.5 to about 5 kilobases (kb); (ii) the length of the RHA is about 0.5 to about 5 kb; or (iii) any combination of (i) and (ii). In certain embodiments, (i) the length of the LHA is about 1.5 kb; (ii) the length of the RHA is about 1.5 kb; or (iii) any combination of (i) and (ii). In certain embodiments, the LHR and/or RHA can be as short as 50 bases.

In certain embodiments, the length of the LHA is about 0.5 to about 5 kilobases (kb). In certain embodiments, the length of the LHA is about 0.5 kb. In certain embodiments, the length of the LHA is about 1 kb. In certain embodiments, the length of the LHA is about 1.5 kb. In certain embodiments, the length of the LHA is about 2 kb. In certain embodiments, the length of the LHA is about 2.5 kb. In certain embodiments, the length of the LHA is about 3 kb. In certain embodiments, the length of the LHA is about 3.5 kb. In certain embodiments, the length of the LHA is about 4 kb. In certain embodiments, the length of the LHA is about 4.5 kb. In certain embodiments, the length of the LHA is about 5 kb. In certain embodiments, the LHR can be as short as 50 bases.

In certain embodiments, the length of the RHA is about 0.5 to about 5 kilobases (kb). In certain embodiments, the length of the RHA is about 0.5 kb. In certain embodiments, the length of the RHA is about 1 kb. In certain embodiments, the length of the RHA is about 1.5 kb. In certain embodiments, the length of the RHA is about 2 kb. In certain embodiments, the length of the RHA is about 2.5 kb. In certain embodiments, the length of the RHA is about 3 kb. In certain embodiments, the length of the RHA is about 3.5 kb. In certain embodiments, the length of the RHA is about 4 kb. In certain embodiments, the length of the RHA is about 4.5 kb. In certain embodiments, the length of the RHA is about 5 kb. In certain embodiments, the RHA can be as short as 50 bases.

In certain embodiments, the length of each of the LHA and RHA is about 0.5 kb. In certain embodiments, the length of each of the LHA and RHA is about 1 kb. In certain embodiments, the length of each of the LHA and RHA is about 1.5 kb. In certain embodiments, the length of each of the LHA and RHA is about 2 kb. In certain embodiments, the length of each of the LHA and RHA is about 2.5 kb. In certain embodiments, the length of each of the LHA and RHA is about 3 kb. In certain embodiments, the length of each of the LHA and RHA is about 3.5 kb. In certain embodiments, the length of each of the LHA and RHA is about 4 kb. In certain embodiments, the length of each of the LHA and RHA is about 4.5 kb. In certain embodiments, the length of each of the LHA and RHA is about 5 kb. In certain embodiments, the LHR and RHA can be as short as 50 bases.

In certain embodiments, the length of each of the left and the right homology arms is sufficient to allow specific recombination into chromosomal DNA of a bird. In one embodiment, the LHA and/or the RHA are at least 500 nucleotides long, for example, between 500-3000 nucleotides long. Typically, the required size of the LHA and/or the RHA homology arms relies on the length of the cassettes which are flanked by these arms. Smaller cassettes require shorter arms and vice versa. In one embodiment, the homology arm can be as short as 50 bases.

In certain embodiments, (i) the LHA is substantially homologous to a corresponding first nucleotide sequence located in an openly transcribed region on chromosome Z of a bird; (ii) the RHA is substantially homologous to a corresponding second nucleotide sequence located in an openly transcribed region on chromosome Z of a bird; or (iii) both (i) and (ii).

As it would be apparent to those skilled in the art, a first sequence is “substantially homologous” to a second sequence if the first sequence and the second sequence are similar or identical in sequence, as long as the first sequence and the second sequence can replace one another by homologous recombination. Method to test and identify homologous recombination are well-known in the art.

In certain embodiments, the first nucleotide sequence in the LHA is 50% to 100% identical in sequence to a first corresponding nucleotide sequence on chromosome Z. In certain embodiments, the first nucleotide sequence in the LHA is 80% to 100% identical in sequence to a first corresponding nucleotide sequence on chromosome Z. In certain embodiments, the first nucleotide sequence in the LHA is 85% to 100% identical in sequence to a first corresponding nucleotide sequence on chromosome Z. In certain embodiments, the first nucleotide sequence in the LHA is 90% to 100% identical in sequence to a first corresponding nucleotide sequence on chromosome Z. In certain embodiments, the first nucleotide sequence in the LHA is 95% to 100% identical in sequence to a first corresponding nucleotide sequence on chromosome Z. In certain embodiments, the first nucleotide sequence in the LHA is 99% to 100% identical in sequence to a first corresponding nucleotide sequence on chromosome Z. In certain embodiments, the first nucleotide sequence in the LHA is 100% identical in sequence to a first corresponding nucleotide sequence on chromosome Z.

In certain embodiments, the fourth nucleotide sequence in the RHA is 50% to 100% identical in sequence to a second corresponding nucleotide sequence on chromosome Z. In certain embodiments, the fourth nucleotide sequence in the RHA is 80% to 100% identical in sequence to a second corresponding nucleotide sequence on chromosome Z. In certain embodiments, the fourth nucleotide sequence in the RHA is 85% to 100% identical in sequence to a second corresponding nucleotide sequence on chromosome Z. In certain embodiments, the fourth nucleotide sequence in the RHA is 90% to 100% identical in sequence to a second corresponding nucleotide sequence on chromosome Z. In certain embodiments, the fourth nucleotide sequence in the RHA is 95% to 100% identical in sequence to a second corresponding nucleotide sequence on chromosome Z. In certain embodiments, the fourth nucleotide sequence in the RHA is 99% to 100% identical in sequence to a second corresponding nucleotide sequence on chromosome Z. In certain embodiments, the fourth nucleotide sequence in the RHA is 100% identical in sequence to a second corresponding nucleotide sequence on chromosome Z.

In certain embodiments, the LHA and/or the RHA is homologous or show homology or identity of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to at least one nucleotide sequence within a target loci within chromosome Z of a bird that serves as the integration site.

A person of skill in the art would understand that the term “openly transcribed region in a chromosome” generally refers to regions of the chromosome which include genes that are transcribed in a level sufficient to allow other genes to be easily transcribed as well. Non-limiting examples of openly transcribed regions are regions in proximity to house-keeping genes which are highly transcribed during the life of the cell or the organism. Other non-limiting examples of openly transcribed regions are regions in-between loci (e.g. chromatin regulatory elements, non-coding DNA, “junk DNA”, etc.). Non-limiting examples of poorly transcribed regions are regions at the ends of each chromosomes, called telomers, which are not transcribed during the life of the cell or the organism. In certain embodiments, the openly transcribed region is located at or downstream to the Hint1Z gene on chromosome Z of a bird.

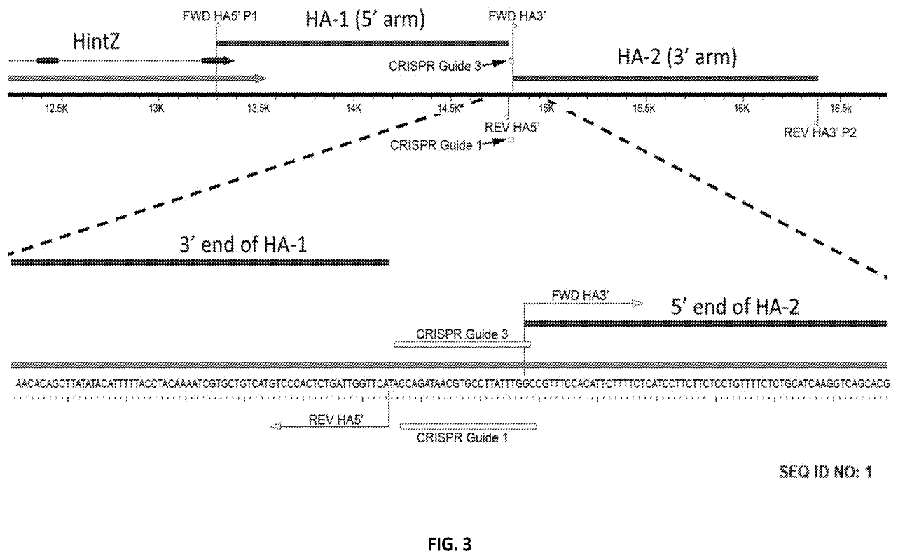

In one embodiment, the LHA and/or the RHA correspond to a genomic sequence which is present on the Z chromosome in birds. In certain embodiments, the genomic sequence is located at or downstream of a gene which is transcriptionally active (for example, at or downstream to the HintlZ gene (GeneID: 395424)). Another contemplated target is Isl1 (Gene ID 369383), also on chromosome Z, which is expressed starting from early stages of embryogenesis. FIG. 3 illustrates an embodiment of the homology arms on chromosome Z, downstream to the Hint1Z gene.

In certain embodiments, the first 5′ nucleotide of LHA corresponds to chromosome Z, position 44,914,961. In certain embodiments, the first corresponding nucleotide sequence of LHA is located at chromosome Z, position 44,914,961 to position 44,916,456.

In certain embodiments, the first 5′ nucleotide of LHA corresponds to Gallus gallus chromosome Z, Assembly GRCg6a, NC_006127.5, position 44,914,961. In certain embodiments, the first corresponding nucleotide sequence of LHA is located at Gallus gallus chromosome Z, Assembly GRCg6a, NC 006127.5, position 44,914,961 to position 44,916,456.

In certain embodiments, the first 5′ nucleotide of RHA corresponds to chromosome Z, position 44,916,480. In certain embodiments, the second corresponding nucleotide sequence of RHA is located at chromosome Z, position 44,916,480 to position 44,918,043.

In certain embodiments, the first 5′ nucleotide of RHA corresponds to Gallus gallus chromosome Z, Assembly GRCg6a, NC_006127.5, position 44,916,480. In certain embodiments, the second corresponding nucleotide sequence of RHA is located at Gallus gallus chromosome Z, Assembly GRCg6a, NC_006127.5, position 44,916,480 to position 44,918,043.

In certain embodiments, (i) the LHA comprises the nucleotide sequence set forth in SEQ ID NO: 105, or a fragment thereof, e.g. at least 50, or at least 500 consecutive nucleotides of the nucleotide sequence set forth in SEQ ID NO: 105; (ii) the RHA comprises the nucleotide sequence set forth in SEQ ID NO: 106, or a fragment thereof, e.g. at least 50, or at least 500 consecutive nucleotides of the nucleotide sequence set forth in SEQ ID NO: 106; or (iii) both (i) and (ii). In certain embodiments, (i) the LHA comprises the nucleotide sequence set forth in SEQ ID NO: 105, or a fragment of at least 1000 consecutive nucleotides of the nucleotide sequence set forth in SEQ ID NO: 105; (ii) the RHA comprises the nucleotide sequence set forth in SEQ ID NO: 106, or a fragment of at least 1000 consecutive nucleotides of the nucleotide sequence set forth in SEQ ID NO: 106; or (iii) both (i) and (ii).

In certain embodiments, the LHA comprises the nucleotide sequence set forth in SEQ ID NO: 105. In certain embodiments, the LHA comprises at least 50, or at least 500 consecutive nucleotides from the nucleotide sequence set forth in SEQ ID NO: 105. In certain embodiments, the LHA comprises at least 1000 consecutive nucleotides from the nucleotide sequence set forth in SEQ ID NO: 105. In certain embodiments, the LHA comprises 500 consecutive nucleotides from the nucleotide sequence set forth in SEQ ID NO: 105. In certain embodiments, the LHA comprises 1000 consecutive nucleotides from the nucleotide sequence set forth in SEQ ID NO: 105.

In certain embodiments, the RHA comprises the nucleotide sequence set forth in SEQ ID NO: 106. In certain embodiments, the RHA comprises at least 50, or at least 500 consecutive nucleotides from the nucleotide sequence set forth in SEQ ID NO: 106. In certain embodiments, the RHA comprises at least 1000 consecutive nucleotides from the nucleotide sequence set forth in SEQ ID NO: 106. In certain embodiments, the RHA comprises 500 consecutive nucleotides from the nucleotide sequence set forth in SEQ ID NO: 106. In certain embodiments, the RHA comprises 1000 consecutive nucleotides from the nucleotide sequence set forth in SEQ ID NO: 106.

In certain embodiments, LHA comprises the nucleotide sequence set forth in SEQ ID NO: 105, or a fragment of at least 50, or at least 500 consecutive nucleotides of the nucleotide sequence set forth in SEQ ID NO: 105; and the RHA comprises the nucleotide sequence set forth in SEQ ID NO: 106, or a fragment of at least 50, or at least 500 consecutive nucleotides of the nucleotide sequence set forth in SEQ ID NO: 106. In certain embodiments, LHA comprises the nucleotide sequence set forth in SEQ ID NO: 105, or a fragment of at least 1000 consecutive nucleotides of the nucleotide sequence set forth in SEQ ID NO: 105; and the RHA comprises the nucleotide sequence set forth in SEQ ID NO: 106, or a fragment of at least 1000 consecutive nucleotides of the nucleotide sequence set forth in SEQ ID NO: 106. In certain embodiments, LHA comprises the nucleotide sequence set forth in SEQ ID NO: 105; and the RHA comprises the nucleotide sequence set forth in SEQ ID NO: 106.

### Optogenetic-Inducible Element

### Promoters

As it would be apparent to those skilled in the art, a “promoter functionally linked to a nucleotide sequence” encompasses that the promotor is located upstream to and is involved in cis in the transcription of the nucleotide sequence. A non-limiting example of a promoter functionally linked to a nucleotide sequence is the first promoter in the OIE which drives the transcription of the nucleotide sequence in the OIE encoding an inducer-activated site-specific recombinase enzyme.

In certain embodiments, the first promoter is a constitutive promoter in birds. As it would be apparent to those skilled in the art, a “constitutive promoter” encompasses a promoter that allows for continual transcription of its associated nucleotide sequence or gene.

In certain embodiments, the first promoter is an inducible promoter in birds. As it would be apparent to those skilled in the art, an “inducible promoter” encompasses a promoter that allows for non-continual transcription of its associated nucleotide sequence or gene. In certain embodiments, the non-continual transcription is modulated by an inducer. In certain embodiments, the inducer is exogenous to a cell of a bird.

The DNA editing agent of this aspect of the present invention will typically comprise a promoter which is operatively linked to drive the expression of the lethality protein or the endonuclease enzyme.

Examples of promoters that may be used in the DNA editing agent include but are not limited to PGK promoter of lentiviruses, CMV promoter, human Synapsin I promoter (hSyn), and the CAG promoter as in the pCAGGS expression vector.

Irrespective of whether the DNA editing agent encodes an endonuclease or a lethality protein, expression of these effector proteins are regulated by a switch which is comprised (e.g. encoded) in the DNA editing agent.

In one embodiment, the first nucleic acid sequence may encode a lethality protein which is operatively linked to a nucleotide sequence encoding a switch that controls the expression of the lethality protein, wherein the switch is regulated by an inducer.

As used herein, the term “switch” refers to a single component or a set of components that act in a coordinated manner to affect a change, encompassing all aspects of biological function such as activation, repression, enhancement or termination of that function. In one embodiment, switches relate to inducible and repressible systems used in gene regulation. In general, an inducible system may be off unless there is the presence of some molecule or energy form (called an inducer) that allows for gene expression. The molecule is said to “induce expression”. The manner by which this happens is dependent on the control mechanisms as well as differences in cell type. A repressible system is on except in the presence of some molecule or energy form (called a corepressor) that suppresses gene expression. The molecule is said to “repress expression”. The manner by which this happens is dependent on the control mechanisms as well as differences in cell type.

The term “inducible” as used herein may encompass all aspects of a switch irrespective of the molecular mechanism involved. Accordingly, a switch as comprehended by the invention may include but is not limited to antibiotic based inducible systems, electromagnetic energy based inducible systems, small molecule based inducible systems, nuclear receptor based inducible systems and hormone based inducible systems. In some embodiments the switch may be a tetracycline (Tet)/DOX inducible system, a light inducible systems, a Abscisic acid (ABA) inducible system, a cumate repressor/operator system, a 40 HT/estrogen inducible system, an ecdysone-based inducible systems or a FKBP12/FRAP (FKBP12-rapamycin complex) inducible system.

It will be appreciated that the inducer should be able to penetrate the egg of the bird. Furthermore, the inducer itself should not be toxic to or alter the development of the embryo inside the egg.

Exemplary inducers contemplated by the present invention include, but are not limited to heat, ultrasound, electromagnetic energy and a chemical. The inducers may be delivered to the egg during the process of egg production inside the hen's body prior to oviposition.

According to a specific embodiment, the switch is induced using electromagnetic energy (for example a component of visible light). The component of visible light may have a wavelength in the range of 450 nm-700 nm or between 450 nm-500 nm, i.e. blue light. The blue light may be of intensity of at least 0.2 mW/cm², or of at least 4 mW/cm².

The component of visible light may have a wavelength in the range of 620 nm-700 nm, i.e. red light.

Single or multiple applications of visible light, in any order and in any combination are contemplated. The visible light may be delivered as single or multiple continuous applications, or as pulses (pulsatile delivery).

Examples of such optogenetic switches are described in Muller et al., Biol Chem. 2015 Feb; 396(2):145-52. doi: 10.1515/hsz-2014-0199; Motta Mena et al., Nat Chem Biol. 2014 March; 10(3): 196-202; and WO 2014018423, the contents of each being incorporated herein by reference.

Other methods of energy activation are contemplated, in particular, electric field energy and/or ultrasound which have a similar effect. If necessary, the proteins pairings of the switch may be altered and/or modified for maximal effect by another energy source.

In one embodiment, the switch drives the expression of an effector molecule which is expressed when the switch is turned on using the inducer. Exemplary promoters for driving the expression of the effector molecule include but are not limited to PGK promoter of lentiviruses, the pCAGG promoter, CMV promoter, EF1-a promoter, and the human Synapsin I promoter (hSyn).

According to a particular embodiment the effector molecule is a site specific recombinase.

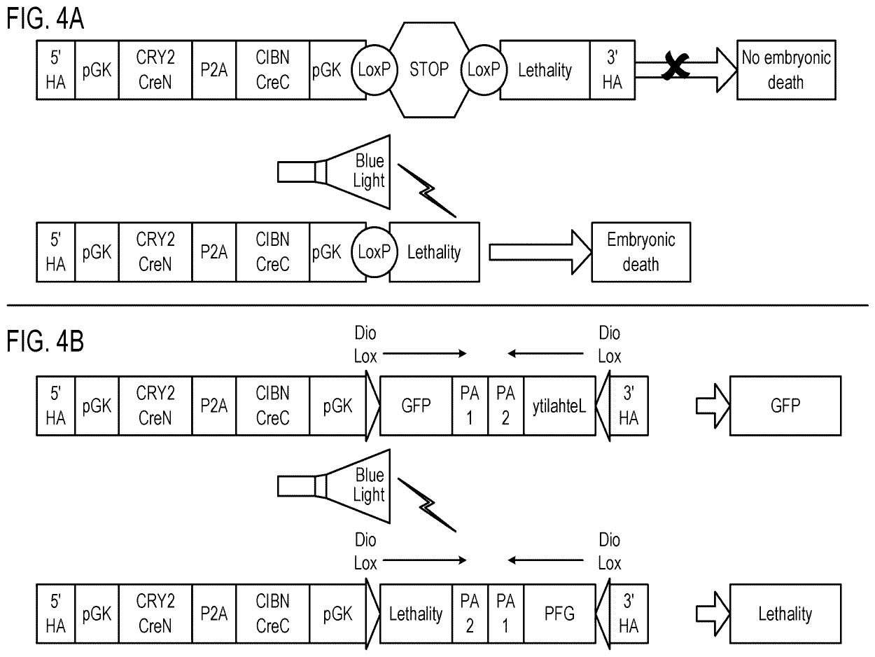

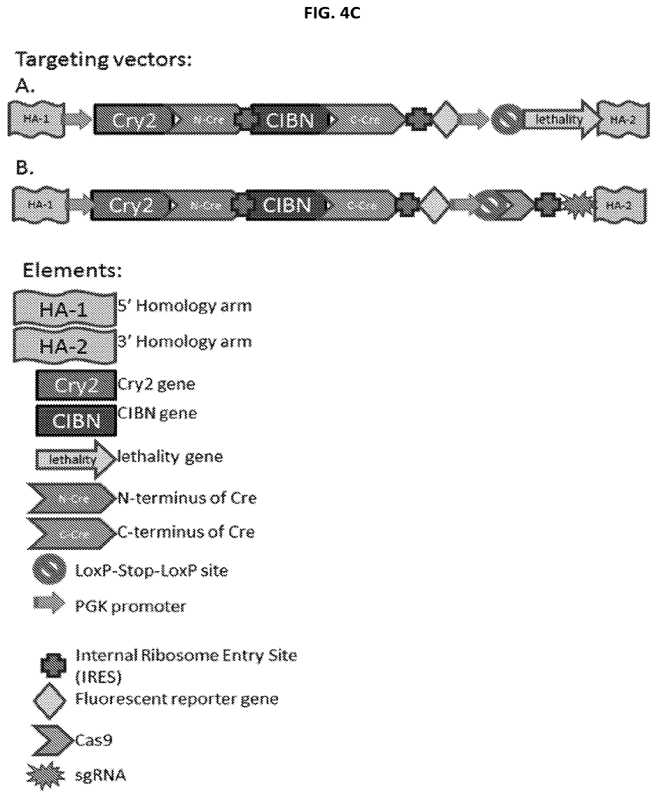

Exemplary optogenetic switches are illustrated in FIGS. 4A-C, each of which utilize the light-sensitive dimerizing protein domains cryptochrome 2 (CRY2) and CIB1 from Arabidopsis thaliana and a site specific recombinase as the effector molecule. The CRY2 is fused in frame to one half of a Cre recombinase whereas the CIB1 is fused in frame to the other half of a Cre recombinase—i.e. a split recombinase enzyme. Thus, when the inducer is provided (blue light is shone), the CRY2 and the CIB1 heterodimerize producing a functional Cre recombinase which is able to carry out site specific recombination.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively.

Additionally, contemplated recombinase recognition sites include, but are not limited to Lox511, Lox5171, Lox2272, m2, Lox71, Lox66, FRT, F₁, F₂, F₃, F₄, F₅, FRT(LE), FRT(RE), attB, attP, attL, and attR.

For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and re-ligation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences. An exemplary targeting vector using the Cre/Lox recombination is illustrated in FIGS. 4A and C. An exemplary targeting vector using the Flp/FRT recombination is illustrated in FIGS. 4B.

### Inducer-Activated Site-Specific Recombinase

In certain embodiments, the inducer-activated site-specific recombinase enzyme can be Cre recombinase (Cre) (SEQ ID NO: 113), or Mag (SEQ ID NO: 114 and SEQ ID NO: 65).

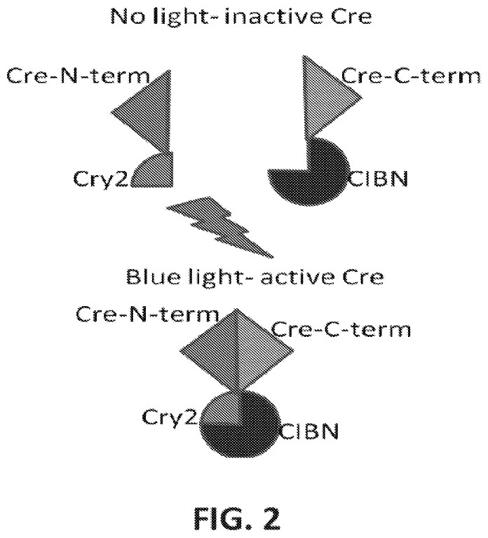

According to certain embodiments, the inducer initiates or increases the transcription of the nucleotide sequence encoding the inducer-activated site-specific recombinase enzyme. In other embodiments, the inducer initiates or increases the translation of the mRNA coding for the inducer-activated site-specific recombinase. In yet other embodiments, the inducer initiates or increases the activity of the inducer-activated site-specific recombinase enzyme. According to certain embodiments, the inducer initiates or increases the formation of a functional inducer-activated site-specific recombinase enzyme by complexing non-functional fragments of site-specific recombinase to each other. As it would be apparent to those skilled in the art, a plurality of non-functional fragments (peptides) of an enzyme, where the enzyme is found in nature as a polypeptide, may co-interact to form a functional enzyme, despite each fragment (peptide) not being covalently linked to other fragments (peptides) as illustrated in FIG. 2. In certain embodiments, the inducer-activated site-specific recombinase comprises non-functional peptide fragments of an inducer-activated site-specific recombinase that combine to form an active inducer-activated site-specific recombinase enzyme in the presence of the inducer.

In certain embodiments, the expression of the inducer-activated site-specific recombinase enzyme is induced by an inducer. In certain embodiments, the inducer is electromagnetic energy. In certain embodiments, the electromagnetic energy is visible light of a wavelength of 380-740 nm, or a component of visible light. In certain embodiments, the component of visible light is blue light of a wavelength of 450-485 nm.

In one embodiment, the switch drives the expression of an effector molecule which is expressed when the switch is turned on using the inducer. Exemplary promoters for driving the expression of the effector molecule include but are not limited to PGK promoter of lentiviruses, the pCAGG promoter, CMV promoter, EFl-a promoter, and the human Synapsin I promoter (hSyn).

According to a particular embodiment the effector molecule is a site specific recombinase.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 and 500 milliseconds, preferably between 1 and 100 milliseconds. The electric field may be applied continuously or in a pulsed manner for 5 about minutes. As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Notably, an alternative approach can be used, in which the activating enzyme (i.e. recombinase enzyme such as Cre for example) is separated from the lethality gene cassette. In this case, the activating enzyme is inserted into the genome of either the male or female bird and the in-active lethality cassette is inserted on the Z chromosome of the corresponding sex of the bird. In this case, the activation of lethality in male embryos is performed merely by crossing the two transgenic parents.

### Lethality-Inducing Element

As it would be apparent to those skilled in the art, a “first protein operatively linked to the activity of a second protein” encompasses that the second protein controls the operation of the first protein in trans. A non-limiting example of a first protein operatively linked to the activity of a second protein is the lethality-promoting protein encoded in the LIE which becomes active by the activity of the inducer-activated site-specific recombinase enzyme encoded in the OIE. The terminology of “in cis” and “in trans” is well accepted and understood to a person of ordinary skill in the art.

In some embodiments, as the inducer-activated site-specific recombinase enzyme activates the lethality-promoting protein in trans, the respective position of the OIE and the LIE in the same molecule, or in different molecules, may be different in different embodiments. In certain embodiments, the OIE and the LIE are in the same molecule. In certain embodiments, the OIE and the LIE are in different molecules.

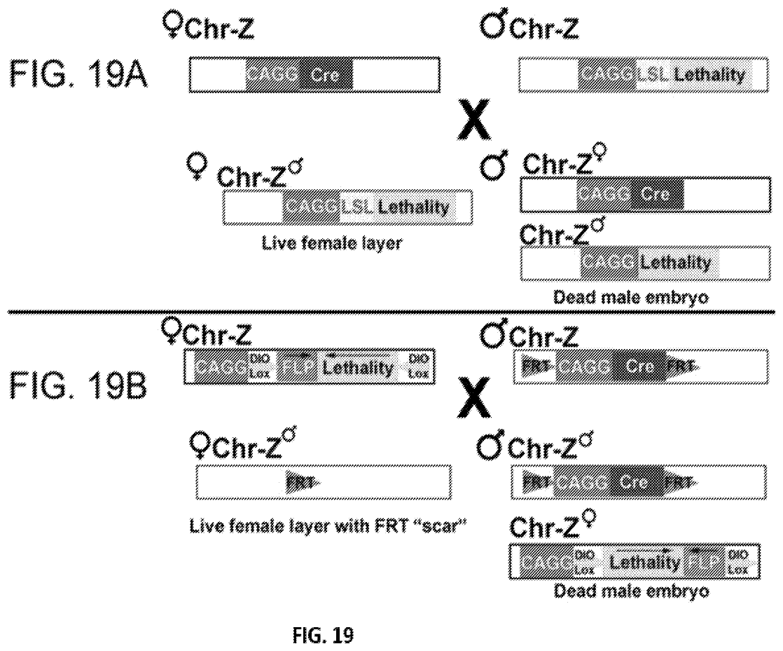

In certain embodiments, the activating enzyme (e.g. recombinase enzyme, such as Cre) is separated from the lethality gene cassette. In this case, the activating enzyme is inserted into the genome of either the male or female bird and the inactive lethality cassette is inserted on the Z chromosome of the corresponding sex of the bird. In this case, the activation of lethality in male embryos is performed merely by crossing the two transgenic parents. FIGS. 19A-19B are embodiments of targeting vectors in which the activating enzyme (Cre for example) is separated from the lethality gene cassette.

In certain embodiments, the LIE further comprises a second promoter functionally linked to the third nucleotide sequence encoding the lethality-promoting protein. In certain embodiments, the second promoter is a constitutive promoter in birds. In certain embodiments, the second promoter is an inducible promoter in birds. In certain embodiments, the second promoter can be pCAGG (SEQ ID NO: 100), pGK (SEQ ID NO: 109), pCMV (SEQ ID NO: 110), phSyn (SEQ ID NO: 111), or pEFI-a (SEQ ID NO: 112).

### Lethality-Promoting Protein

As used herein, the term “lethality protein” refers to a protein that is lethal to an avian embryo (e.g. male embryo) thus preventing the hatching of a live male bird from the egg. Examples of lethality proteins include, but are not limited to a toxin, a cytotoxic protein, pro-apoptotic protein, a BMP antagonist, an inhibitor of Wnt signaling and an FGF antagonist.

Exemplary toxins contemplated by the present invention include, but are not limited to Pseudomonas exotoxin (GenBank Accession No. ABU63124), diphtheria toxin (GenBank Accession No. AAV70486) and ricin toxin (GenBank Accession No. EEF27734).

In certain embodiments, the lethality-inducing protein can be a toxin, a pro-apoptotic protein, an inhibitor of the Wingless/Integrated (Wnt) signaling pathway, a bone morphogenetic protein (BMP) antagonist, a fibroblast growth factor (FGF) antagonist, or a lethality-inducing fragment thereof. In certain embodiments, the lethality-inducing protein is a toxin or a lethality-inducing fragment thereof. In certain embodiments, the lethality-inducing protein is a pro-apoptotic protein or a lethality-inducing fragment thereof. In certain embodiments, the lethality-inducing protein is an inhibitor of the Wnt signaling pathway or a lethality-inducing fragment thereof. In certain embodiments, the lethality-inducing protein is a BMP antagonist or a lethality-inducing fragment thereof. In certain embodiments, the lethality-inducing protein is an FGF antagonist or a lethality-inducing fragment thereof. As it would be apparent to those skilled in the art, a “lethality-inducing fragment” of a molecule is any fragment of a molecule that suffices to induce lethality.

In certain embodiments, lethality is mediated by a RNA-guided DNA endonuclease enzyme. In certain embodiments, the DNA editing agent further comprises a nucleotide sequence that encodes for a guide RNA that targets an essential gene of the bird, this nucleotide sequence being operatively linked to the activity of the inducer-activated site-specific recombinase enzyme. In certain embodiments, the essential gene can be bone morphogenetic protein receptor, type IA (BMPR1A, Gene ID: 396308), bone morphogenetic protein 2 (BMP2, Gene ID: 378779), bone morphogenetic protein 4 (BMP4, Gene ID: 396165), or fibroblast growth factor receptor 1 (FGFR1, Gene ID: 396516).

As it would be apparent to those skilled in the art, a “RNA-guided DNA endonuclease enzyme” encompasses a DNA endonuclease enzyme which unwinds DNA and looks for sites complementary to a guide RNA molecule. In certain embodiments, the guide RNA molecule comprises the nucleotide sequence set forth in one of SEQ ID NOs:66-77.

In some embodiments, the nucleic acid sequence further comprises a targeting sequence (or guide sequence) which targets the endonuclease to disrupt a gene which is essential for survival of the embryo. In this way, a lethal phenotype is brought about in the embryo and a live (male) chick cannot hatch from the egg.

### Safe Lock Element

In one embodiment, the DNA editing agent disclosed herein may further comprise a “safe-lock” element which ensures the optogenic-lethality mechanism is inactive until the safe-lock element is removed. This element basically renders the optogenic-lethality system inactive by default. The optogenic-lethality system would become active only when the genome-edited cells are exposed to an agent which could remove the safe-lock element from the DNA editing agent. This “safe-lock” mechanism ensures that throughout the production process, cells which have undergone HR need not be protected from light, since the optogenic system is essentially inactive.

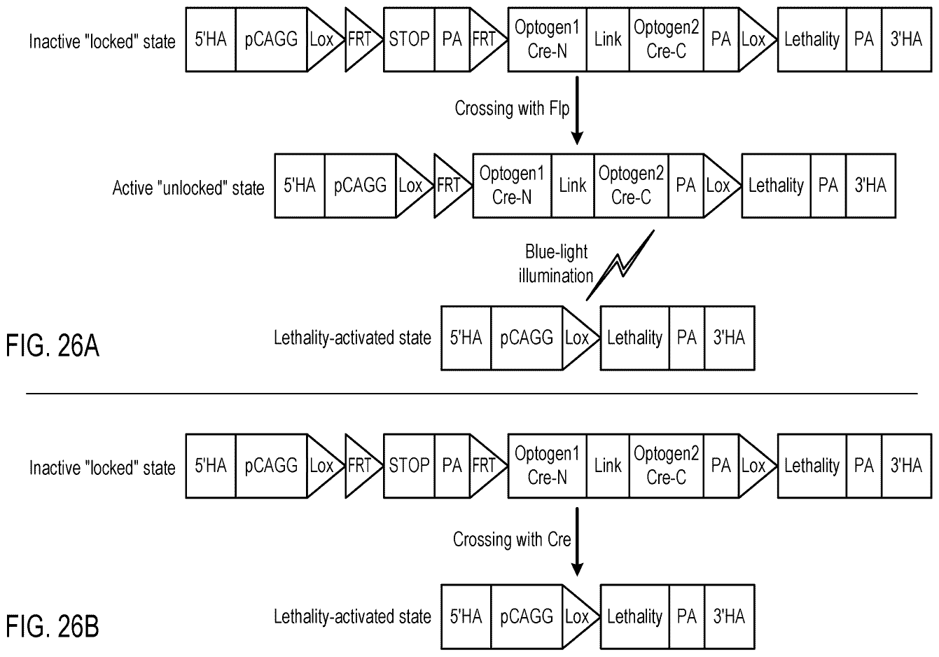

In one embodiment, the safe-lock element is inserted downstream to the promoter in the OIE but upstream of the sequence encoding the inducer-activated site-specific recombinase. The safe-lock element comprises nucleotide sequences (STOP element) that prevent transcription of the inducer-activated site-specific recombinase encoded by the OIE (see FIG. 26A, inactive “locked” state). In one embodiment, the safe-lock element comprises a coding sequence for a protein followed by a polyadenylation site. In other embodiments, any other sequences which will prevent the transcription of downstream coding sequences can also be used as a safe lock element. In one embodiment, the safe-lock element is flanked by two FRT sites. Thus, this safe-lock element can be removed upon the expression of the Flp recombinase. Once the safe-lock element is removed, the optogenes encoded in the OIE could be transcribed and become active in a light-dependent manner (FIG. 26A, active “unlocked” state).

In another embodiment, the safe-lock element and the sequence encoding the inducer-activated site-specific recombinase are flanked by the Lox sequences (FIG. 26B, inactive “locked” state). Upon expressing the Cre recombinase, the safe-lock element as well as the sequence encoding the inducer-activated site-specific recombinase could be removed, thus allowing the expression of the lethality-promoting protein encoded in the LIE (FIG. 26B, lethality-activated state).

### Chimeric Chicks

As used herein, the term “chimeric”, “chimera” or “chimeric chick” refers to a bird cell that contains the DNA editing agent disclosed herein, or a bird that has cells containing the DNA editing agent disclosed herein. It should also be noted that chimera embryos or chimera adult birds can also be referred as “surrogate”; hence, these terms can be used interchangeably. Representative examples of chimeric bird cells include, but are not limited to, bird primordial germ cells (PGCs) such as gonadal PGCs, blood PGCs, germinal crescent PGCs, or gametes that contain the DNA editing agent disclosed herein. Representative examples of chimeric bird include, but are not limited to, chicken, turkey, duck, geese, quail, pheasant, or ostrich that has cells containing the DNA editing agent disclosed herein.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

In one embodiment, there is provided a bird cell comprising an exogenous polynucleotide cassette as disclosed herein. In some embodiments, the cassette comprises a formula 5′-OIE-LIE-3′ or a formula 5′-LIE-OIE-3′, wherein (i) the OIE is optogenetic-inducible element, comprising a first promoter functionally linked to a second nucleotide sequence encoding an inducer-activated site-specific recombinase enzyme; and (ii) the LIE is a lethality-inducing element, comprising a third nucleotide sequence encoding a lethality-promoting protein, operatively linked to the activity of the inducer-activated site-specific recombinase enzyme. The exogenous polynucleotide cassette is stably integrated into the Z chromosome of the cell.

Further provided, in another aspect, is a bird cell population, comprising bird cells that comprise an exogenous polynucleotide cassette as disclosed herein. In some embodiments, the cassette comprises a formula 5′-OIE-LIE-3′ or a formula 5′-LIE-OIE-3′, wherein (i) the OIE is optogenetic-inducible element, comprising a first promoter functionally linked to a second nucleotide sequence encoding an inducer-activated site-specific recombinase enzyme; and (ii) the LIE is a lethality-inducing element, comprising a third nucleotide sequence encoding a lethality-promoting protein, operatively linked to the activity of the inducer-activated site-specific recombinase enzyme. The exogenous polynucleotide cassette is stably integrated into the Z chromosome of the cell.

In certain embodiments, the cells of the bird comprise an exogenous polynucleotide cassette, comprising a formula 5′-LHA-OIE-LIE-RHA-3′ or a formula 5′-LHA-LIE-OIE-RHA-3′, wherein (i) the LHA is left homology arm, comprising a first nucleotide sequence that is substantially homologous to a first corresponding nucleotide sequence on chromosome Z of a bird; (ii) the OIE is optogenetic-inducible element, comprising a first promoter functionally linked to a second nucleotide sequence encoding an inducer-activated site-specific recombinase enzyme; (iii) the LIE is lethality-inducing element, comprising a third nucleotide sequence encoding a lethality-promoting protein, operatively linked to the activity of the inducer-activated site-specific recombinase enzyme; and (iv) the RHA is right homology arm, comprising a fourth nucleotide sequence, that is substantially homologous to a second corresponding nucleotide sequence on chromosome Z of a bird.

In certain embodiments, the cells of the bird comprising the exogenous polynucleotide cassette comprise bird primordial germ cells (PGCs). In certain embodiments, the bird PGCs can be gonadal PGCs, blood PGCs, or germinal crescent PGCs.

As used herein, the terms “primordial germ cell” and “PGC” refer to a diploid cell that is present in the early embryo and that can differentiate/develop into haploid gametes (i.e. spermatozoa and ova) in an adult bird. Primordial germ cells can be isolated from different developmental stages and from various sites in a developing avian embryo as is known to those of skill in the art including, but not limited to the genital ridge, the developing gonad, the blood, and the germinal crescent. See e.g. Chang et al., Cell Biol Int 21:495-9, 1997; Chang et al., Cell Biol Int 19:143-9, 1995; Allioli et al., Dev Biol 165:30-7, 1994; Swift, Am J Physiol 15:483-516; and PCT International Publication No. WO 99/06533. The genital ridge is a section of a developing embryo that is known to a person of ordinary skill in the art. See e.g., Strelchenko, Theriogenology 45: 130-141, 1996; Lavoir, J Reprod Dev 37: 413-424, 1994. Typically, PGCs are stain positively in the periodic acid-Schiff (PAS) technique. In several species, PGCs can be identified using an anti-SSEA antibody (one notable exception being turkeys, the PGCs from which do not display the SSEA antigen). Various techniques for isolation and purification ofPGCs are known in the art, including the concentration of PGCs from blood using Ficoll density gradient centrifugation (Yasuda et al., J Reprod Fertil 96:521-528, 1992).

The in vitro culture of PGCs is possible using a medium containing chicken and bovine serum, conditioned media, feeder cells and growth factors such as FGF2 (van de Lavoir et al. 2006, Nature 441:766-769. doi:10.1038/nature04831); Choi et al. 2010, PLoS ONE 5:e12968. doi:10.1371/journal.pone.0012968; MacDonald et al.2010. PLoS ONE 5:e15518. doi:10.1371/journal.pone.0015518). Recently, it has been shown that a feeder replacement medium containing growth factors to activate the FGF, insulin and TGF-0 signalling pathways could be used to propagate PGCs (Whyte et al. 2015, Stem Cell Rep 5:1171-1182. doi:10.1016/j.stemcr.2015.10.008). Furthermore in this report, use of ovotransferrin as a replacement for the iron-carrying proteins present in avian serum permitted feeder-free and serum-free propagation of PGCs, with cells maintaining a high rate of proliferation.

Primordial germ cells (PGCs) can be provided and formulated for carrying out the presently disclosed subject matter by any suitable technique, and stored, frozen, cultured, or the like prior to use as desired. For example, primordial germ cells can be collected from donor embryos at an appropriate embryonic stage. Stages of avian development are referred to herein by one of two art-recognized staging systems: the Eyal-Giladi & Kochav system (EG&K; see Eyal-Giladi & Kochav, Dev Biol 49:321-327, 1976), which uses Roman numerals to refer to pre-primitive streak stages of development, and the Hamburger & Hamilton staging system (H&H; see e.g., Hamburger & Hamilton, J Morphol 88:49-92, 1951), which uses Arabic numerals to reference to post-laying stages. Unless otherwise indicated, the stages referred to herein are stages as per the H&H staging system.

For example, PGCs can be isolated at stage 4, or the germinal crescent stage, through stage 30, with cells being collected from blood, genital ridge, or gonad in the later stages. The primordial germ cells are, in general, twice the size of somatic cells and easily distinguished and separated therefrom on the basis of size. Male (or homogametic) primordial germ cells (ZZ) can be distinguished from heterogametic primordial germ cells (Zw) by any suitable technique, such as collecting germ cells from a particular donor and typing other cells from that donor, the collected cells being of the same chromosome type as the typed cells.

An alternative to the use of PGCs is the direct transfection of spermatozoa using a DNA editing agent disclosed herein—see for example Cooper et al., 2016 Transgenic Res 26:331-347, doi:10.1007/s 11248-016-0003-0.

To produce chimeric birds from PGCs edited in vitro, the exogenous edited cells are injected intravenously into surrogate host embryos, at a stage when their endogenous PGCs are migrating to the genital ridge. The “donor” PGCs may be of the same species as the surrogate host embryo or of a different species. The edited “donor” PGCs must remain viable and in one embodiment, out-compete the endogenous PGCs if they are to colonise the forming gonad and transmit the edited chromosome(s) through the germline. To provide donor PGCs with an advantage, the number of endogenous PGCs can be reduced by chemical or genetic ablation (Smith et al. 2015, Andrology 3:1035-1049. doi:10.1111/andr.12107). Exposing the blastoderm of surrogate embryos to emulsified Busulfan has been shown to increase germline transmission of donor PGCs to over 90%, though this rate drops significantly if PGCs have been cultured or cryopreserved (Nakamura et al. 2008, Reprod Fertil Dev 20:900-907. doi:10.1071/RD08138; Naito et al. 2015, Anim Reprod Sci. 153:50-61. doi:10.1016/j.anireprosci.2014.12.003). Other methods of skewing the ratio of edited PGCs to native PGCs are described in US Application No. 20060095980.

Optionally, the PGCs may be transplanted into adult gonads as known in the art, see for example Trefil et al., 2017 Sci Rep, October 27; 7(1):14246 doi: 10.1038/s41598-017-14475-w.

The genetically modified cells (e.g. PGCs) can be formulated for administration to other birds by dissociating the cells (e.g., by mechanical dissociation) and intimately admixing the cells with a pharmaceutically acceptable carrier (e.g., phosphate buffered saline solution). The primordial germ cells are in one embodiment gonadal primordial germ cells, and in another embodiment blood primordial germ cells (“gonad” or “blood” referring to their tissue of origin in the original embryonic donor). The primordial germ cells administered can be heterogametic (Zw) or homogametic (ZZ). PGCs can be administered in physiologically acceptable carrier, in one embodiment at a pH of from about 6 to about 8 or 8.5, in a suitable amount to achieve the desired effect (e.g., 100 to 30,000 PGCs per embryo). The PGCs can be administered free of other ingredients or cells, or other cells and ingredients can be administered along with the PGCs.

Administration of the primordial germ cells to the recipient animal in-ovo can be carried out at any suitable time at which the PGCs can still migrate to the developing gonads. In one embodiment, administration is carried out from about stage IX according to the Eyal-Giladi & Kochav (EG&K) staging system to about stage 30 according to the Hamburger & Hamilton staging system of embryonic development, and in another embodiment, at stage 15. For chickens, the time of administration is thus during days 1, 2, 3, or 4 of embryonic development: in one embodiment day 2 to day 2.5. Administration is typically by injection into any suitable target site, such as the region defined by the amnion (including the embryo), the yolk sac, etc. In one embodiment, injection is into the embryo itself (including the embryo body wall), and in alternative embodiments, intravascular or intracoelomic injection into the embryo can be employed. In other embodiments, the injection is performed into the heart. The methods of the presently disclosed subject matter can be carried out with prior sterilization of the recipient bird in ovo (e.g. by chemical treatment using Busulfan of by gamma or X-ray irradiation). As used herein, the term “sterilization” refers to render partially or completely incapable of producing gametes derived from endogenous PGCs). When donor gametes are collected from such a recipient, they can be collected as a mixture with gametes of the donor and the recipient. This mixture can be used directly, or the mixture can be further processed to enrich the proportion of donor gametes therein.

The in-ovo administration of the primordial germ cells can be carried out by any suitable technique, either manually or in an automated manner. In one embodiment, in-ovo administration is performed by injection. The mechanism of in-ovo administration is not critical, but it is the mechanism should not unduly damage the tissues and organs of the embryo or the extraembryonic membranes surrounding it so that the treatment will not unduly decrease hatch rate. A hypodermic syringe fitted with a needle of about 18 to 26 gauge is suitable for the purpose. A sharpened pulled glass pipette with an opening of about 20-50 microns diameter may be used. Depending on the precise stage of development and position of the embryo, a one-inch needle will terminate either in the fluid above the chick or in the chick itself. A pilot hole can be punched or drilled through the shell prior to insertion of the needle to prevent damaging or dulling of the needle. If desired, the egg can be sealed with a substantially bacteria-impermeable sealing material such as wax or the like to prevent subsequent entry of undesirable bacteria. It is envisioned that a high-speed injection system for avian embryos will be particularly suitable for practicing the presently disclosed subject matter. All such devices, as adapted for practicing the methods disclosed herein, comprise an injector containing a formulation of the primordial germ cells as described herein, with the injector positioned to inject an egg carried by the apparatus in the appropriate location within the egg. In addition, a sealing apparatus operatively connected to the injection apparatus can be provided for sealing the hole in the egg after injection thereof. In another embodiment, a pulled glass micropipette can be used to introduce the PGSc into the appropriate location within the egg—for example directly into the blood stream, either to a vein or an artery or directly into the heart.

Once the eggs have been injected with the modified PGCs, the chimeric embryo is incubated to hatch. It is raised to sexual maturity, wherein the chimeric bird produces gametes derived from the donor PGCs.

The gametes, (either eggs or sperm) from the chimeras (or from material that has been directly genetically manipulated, as described herein above) are then used to raise founder chickens (F1). Molecular biology techniques known in the art (e.g. PCR and/or Southern blot) may be used to confirm germ-line transmission. F1 chickens may be back-crossed to generate homozygous ZZ carrier males and carrier females (F2).

Gametes from founder chickens—F2, can then be used to expand the breeding colonies. The colonies are typically grown until sexual maturity. Fertile eggs obtained from these flocks may be tested for early embryonic mortality of the males by exposure to an inducer which elicits the lethal phenotype, (e.g. blue light illumination). Following induction, the eggs are incubated (for example for 8 days) and screened (e.g. by light-candling) to detect for early embryonic mortality.

In one embodiment, there is provided a method of generating a chimeric bird, comprising administering to a bird cell population an exogenous polynucleotide cassette. In some embodiments, the cassette has a formula 5′-OIE-LIE-3′ or a formula 5′-LIE-OIE-3′, wherein (i) the OIE is optogenetic-inducible element, comprising a first promoter functionally linked to a second nucleotide sequence encoding an inducer-activated site-specific recombinase enzyme; and (ii) the LIE is a lethality-inducing element, comprising a third nucleotide sequence encoding a lethality-promoting protein, operatively linked to the activity of the inducer-activated site-specific recombinase enzyme. The exogenous polynucleotide cassette is stably integrated into the Z chromosome of the cells. These genome-edited cells are then injected into a recipient bird embryo.

In another embodiment, there is provided a method of generating a chimeric bird, comprising administering to a bird cell population an exogenous polynucleotide cassette. In some embodiments, the cassette has a formula 5′-LHA-OIE-LIE-RHA-3′ or a formula 5′-LHA-LIE-OIE-RHA-3′, wherein (i) the LHA is left homology arm, comprising a first nucleotide sequence that is substantially homologous to a first corresponding nucleotide sequence on chromosome Z of a bird; (ii) the OIE is optogenetic-inducible element, comprising a first promoter functionally linked to a second nucleotide sequence encoding an inducer-activated site-specific recombinase enzyme; (iii) the LIE is lethality-inducing element, comprising a third nucleotide sequence encoding a lethality-promoting protein, operatively linked to the activity of the inducer-activated site-specific recombinase enzyme; and (iv) the RHA is right homology arm, comprising a fourth nucleotide sequence, that is substantially homologous to a second corresponding nucleotide sequence on chromosome Z of a bird. The exogenous polynucleotide cassette is stably integrated into the Z chromosome of the cells. These genome-edited cells are then injected into a recipient bird embryo.

In certain embodiments, the method further comprises incubating the chimeric bird embryo, in-ovo, until hatching. In certain embodiments, the method further comprises raising the chimeric bird to sexual maturity, wherein the chimeric bird produces gametes derived from the administered cells.

In certain embodiments, the genome-edited cells are administered by in-ovo injection. In another embodiment, the embryos can be taken out of the egg-shell, injected “ex-ovo” and then placed back in a surrogate egg-shell. In certain embodiments, the administrated cell population is derived from the same avian species as the recipient bird embryo. In certain embodiments, the administrated cell population is derived from a different avian species as the recipient bird embryo.

In another embodiment, the chimeric bird can be generated by injecting the genome-edited PGCs to blastoderm. In general, the genome-edited PGCs can be returned or injected back to where endogenous PGCs are located. In one embodiment, the genome-edited PGCs can be returned back to the blastoderm. Alternatively, the genome-edited PGCs can be returned back to the germinal crescent, blood, embryonic gonads, or even to adult gonads.

In certain embodiments, the genome-edited bird cell population is administered when the recipient embryo is about stage IX according to the Eyal-Giladi & Kochav staging system. In certain embodiments, the bird cell population is administered when the recipient embryo is about stage 30 according to the Hamburger & Hamilton staging system. In certain embodiments, the bird cell population is administered when the recipient embryo is about stage IX according to the Eyal-Giladi & Kochav staging system; and about stage 30 according to the Hamburger & Hamilton staging system. In certain embodiments, the bird cell population is administered when the recipient embryo is after stage 14 according to the Hamburger & Hamilton staging system.

In certain embodiments, the genome-edited bird cell population is administered after irradiation of the embryo. In certain embodiments, the irradiation comprises γ-irradiation or X-ray irradiation. In certain embodiments, the irradiation comprises 600-800 rad of γ irradiation. In certain embodiments, the irradiation comprises 600-800 rad of irradiation. In certain embodiments, the irradiation comprises 400-1000 rad of irradiation. In certain embodiments, the irradiation comprises 200-1200 rad of irradiation.

Further provided, in another aspect, is a chimeric bird obtainable from the methods described above.

### Methods of Use

In one embodiment, there is provided a method of generating a cell of a bird, comprising the step of contacting a cell of a bird with an exogenous polynucleotide cassette. In some embodiments, the cassette has a formula 5′-OIE-LIE-3′ or a formula 5′-LIE-OIE-3′ as described herein. The exogenous polynucleotide cassette is stably integrated into the Z chromosome of the cells. In another embodiment, the method comprises the step of contacting the cell of the bird with an exogenous polynucleotide cassette having a formula 5′-LHA-OIE-LIE-RHA-3′ or a formula 5′-LHA-LIE-OIE-RHA-3′ as described herein. The exogenous polynucleotide cassette is stably integrated into the Z chromosome of the cells.

In another embodiment, there is provided a method of inducing lethality in a male embryo in a fertilized egg of a bird, comprising the steps of: administering the DNA editing agent disclosed herein to a population of bird cells to generate genome-edited bird cells; transferring these genome-edited bird cells to recipient bird embryos; and exposing the embryos to an inducer that elicits expression of the lethality-promoting protein encoded by the DNA editing agent, thereby inducing lethality of the male embryo in the fertilized egg of the bird. The various elements of the DNA editing agent, such as optogenetic-inducible element, inducer-activated site-specific recombinase, inducer, lethality-promoting protein, etc have been discussed above.

In one embodiment, the DNA editing agent used in the above method of inducing lethality in a male embryo comprises (i) a LHA comprising the sequence of SEQ ID NO:105, (ii) an OIE comprising the sequence of SEQ ID NO:100, which is connected to the sequence of SEQ ID NO:116, which is connected to the sequence of SEQ ID NO:101, which is connected to the sequence of SEQ ID NO:103, which is connected to the sequence of SEQ ID NO:102, which is connected to the sequence of SEQ ID NO:104, which is connected to the sequence of SEQ ID NO:116, or the OIE comprises the sequence of SEQ ID NO:100, which is connected to the sequence of SEQ ID NO:116, which is connected to the sequence of SEQ ID NO:107, which is connected to the sequence of SEQ ID NO:103, which is connected to the sequence of SEQ ID NO:108, which is connected to the sequence of SEQ ID NO:104, which is connected to the sequence of SEQ ID NO:116, (iii) a LIE comprising the sequence of SEQ ID NO:92, or SEQ ID NO:94, or SEQ ID NO:96, or SEQ ID NO:98, and (iv) a RHA comprising the sequence of SEQ ID NO: 106.

In another embodiment, there is provided a method of inducing lethality in a male embryo in a fertilized egg of a bird, comprising the steps of administering to a population of bird cells the DNA editing agent containing a safe-lock element as disclosed herein, thereby generating genome-edited bird cells; transferring these genome-edited bird cells to recipient bird embryos; and exposing the embryos to an agent that removes the STOP element from the STOP element, thereby eliciting expression of the lethality-promoting protein encoded by the DNA editing agent and inducing lethality of the male embryo in the fertilized egg of the bird. The various elements of the DNA editing agent, such as optogenetic-inducible element, inducer-activated site-specific recombinase, inducer, safe-lock element, lethality-promoting protein, etc have been discussed above. In one embodiment, the DNA editing agent used in the above method of inducing lethality in a male embryo comprises the sequence of one of SEQ ID NOs:120-127.

In one embodiment, removal of the STOP element from the DNA editing agent requires the expression of Flp when the STOP element is flanked by two FRT sites (see FIG. 26A, inactive “locked” state). In one embodiment, expression of Flp may be accomplished by contacting the genome-edited bird cells with a nucleotide sequence encoding the Flp protein (e.g. SEQ ID NO:129 or SEQ ID NO:131). Upon expressing the Flp recombinase, the STOP element inserted between the promoter and the sequence encoding the inducer-activated site-specific recombinase in OLE is removed, thus allowing the expression of the inducer-activated site-specific recombinase (FIG. 26A, active “unlocked” state). Upon further exposure to an inducer (e.g. blue light), the inducer-activated site-specific recombinase would be activated, resulting in the removal of OLE and the expression of the lethality-promoting protein encoded in the LIE (FIG. 26A, lethality-activated state).

In another embodiment, removal of the STOP element may be accomplished by contacting the genome-edited bird cells with a nucleotide sequence encoding the Cre protein (e.g. SEQ ID NO:128 or SEQ ID NO:132). In one embodiment, the safe-lock element and the sequence encoding the inducer-activated site-specific recombinase are flanked by the Lox sequences (FIG. 26B, inactive “locked” state). Upon expressing the Cre recombinase, the safe-lock element as well as the sequence encoding the inducer-activated site-specific recombinase are removed, thus allowing the expression of the lethality-promoting protein encoded in the LIE (FIG. 26B, lethality-activated state).

In another embodiment, the step of lethality induction can be done at any developmental stage, for example soon after oviposition. The sooner death is induced, the earlier the embryo will die. Embryonic mortality is thus achieved thereafter. If a toxin or pro-apoptotic agent is used to induce embryonic death, the embryo dies soon after induction. If LIE is based on a gene that disrupts essential signaling pathway, such as the BMP, for example by expressing the BMP4 antagonist Noggin, cell death will be induced at the developmental time point in which the pathway is active and required. For example, BMP is active during stages comprising blastulation, gastrulation, neurulation, organogenesis. In certain embodiments, lethality is induced during oviposition, and the embryo dies within about 36 hours after exposure to Noggin. As an example, if the optogenetic system is activated soon after oviposition (Stages X-XIII EG&K), the embryo will die at that stage. BMP4 knock-out mice embryos die in-uterus from 6.5 days post coitum (dpc) to ~8.5-9 dpc which is equivalent to the first 30-36 hours of incubation in chicken embryo development. In certain embodiments, the lethality is induced during the 21-day period from fertilization to hatching. In certain embodiments, the lethality is induced more than once during the 21 days from fertilization to hatching. In certain embodiments, the lethality is induced in the egg before early blastulation stages known as stages X-XIII EG&K (Eyal-Giladi and Kochav, 1976).

In certain embodiments, the method is performed in-vivo. In certain embodiments, the method is performed ex-vivo. In certain embodiments, the method is performed in-ovo. In certain embodiments, the method is performed in-vitro.

It is appreciated that certain features of certain embodiments, which are, for clarity, described in the context of separate embodiments, may also be provided in combination in a single embodiment. Conversely, various features of certain embodiments, which are, for brevity, described in the context of a single embodiment, may also be provided separately or in any suitable sub-combination or as suitable in any other described embodiment. Certain features described in the context of various embodiments are not to be considered essential features of those embodiments, unless the embodiment is inoperative without those elements.

Various embodiments and aspects as delineated hereinabove and as claimed in the claims section below find experimental support in the following examples.

### EXAMPLES

Reference is now made to the following examples, which together with the above descriptions illustrate some embodiments in a non-limiting fashion.

Generally, the nomenclature used herein and the laboratory procedures utilized include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

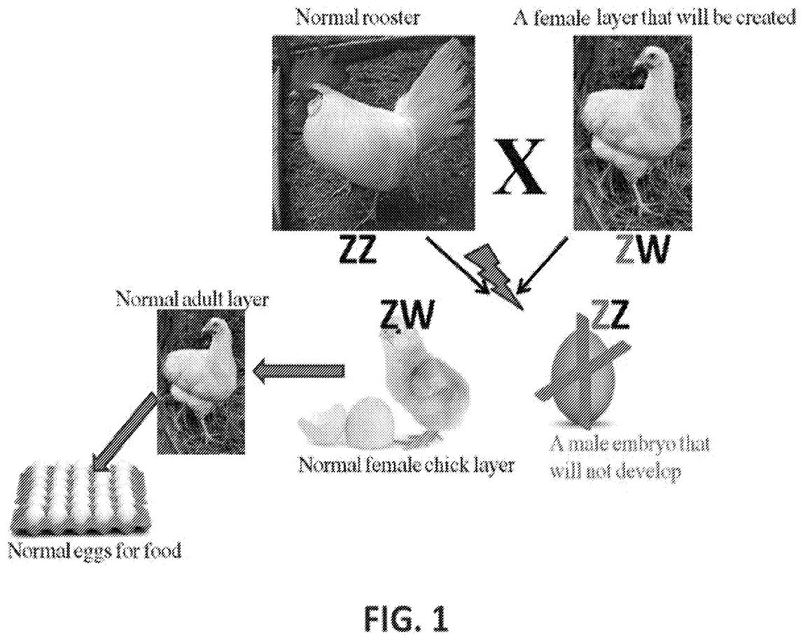

Generating a genome modified chicken line is a multi-step process. The final product is a female layer-hens line which is completely identical, with respect to genome content, to the layer which is used today in the industry (see FIG. 1).

In one embodiment, the workflow comprises 5 main steps: (1) Generating and cultivating chicken Primordial Germ-Cells (PGCs) lines; (2) Genome modification in cultured PGCs; (3) Transplantation of modified PGCs to embryos, and producing chimera chickens that will be screened for germ-line transmission and identifying potential founder carriers; (4) Breeding founder chickens from genetic material obtained from the chimeras; (5) Expanding the founder chickens colonies to founder flocks and re-verifying the germline transmission.

### Materials and Methods

PGC Culture Medium: Avian PGC culture medium consists of DMEM (Gibco) calcium free medium diluted with water to 250 mOsmol/L, containing 12.0 mM glucose, 2.0 mM GlutaMax (Gibco), 1.2 mM pyruvate (Gibco), 1×MEM vitamin (Gibco), 1×B-27 supplement (Gibco), 1×NEAA (Gibco), 0.1 mM β-mercaptoethanol (Gibco), 1×nucleosides (Biological industries), 0.2% ovalbumin (Sigma), 0.1 mg/ml sodium heparin (Sigma), CaCl₂ 0.15 mM (Sigma), 1×MEM vitamin (Gibco), 1×Pen/Strep (Biological industries), 0.2% chicken serum (Sigma) in avian DMEM. The following growth factors were added before use: human Activin A, 25 ng/mL (Peprotech); human FGF2 4 ng/mL (R&D Biosystems), ovotransferin (5 g/ml) (Sigma). AkoDMEM refers to a Diluted medium containing glucose, pyruvate and vitamins.

PGC Line Derivation: PGC lines were derived by placing ~1.0-3.0 μL of blood isolated from stage 14 to 16 (H&H) embryos in 300 μL medium in a 48-well plate. Medium was changed every 2 days. When total cell number reached 1×10⁵, total volume of medium was changed every 2 days and cells were propagated at 2-4×10⁵ cells/ml medium. Cells were frozen in PGC culture medium containing 10% DMSO, temperature was gradually decreased to −80 °C, stored for 1-3 days and transferred to liquid nitrogen.

Sex Determination and PGC Line Characterization: Each PGC line was characterized for sexing, mRNA expression of PGC markers, and protein expression of the known PGCs marker SSEA14. DNA from the donor embryo was isolated and kept for future reference. For sexing, DNA from 2-4×10⁵ PGCs cells was collected, re-suspended in tail buffer (102-T, Viagen) containing 100 μg/ml Proteinase K (Sigma) and incubated at 55 °C for 3 hours. The Proteinase K was inactivated at 85 °C for 45 minutes. PCR for sex determination was performed with primers from W chromosome that target female chromosome (P17, P18) and Ribosomal S18 (P19, P20) as a control. For gene expression analysis, RNA was purified using TRIZOL reagent and 1 μg of RNA was used for cDNA library production by reverse transcription PCR reaction (GoScript Reverse transcriptase, Promega). The cDNA served as a template for PCR by using Dazl, Sox2, cPouV, Nanog, Klf4, cVH primers, P21-P22, P23-P24, P25-P26, P27-P28, P29-P30, P31-P32, respectively.

Immunohistochemistry With Anti SSEA1 Antibody: Cells were collected, fixed with 4% PFA, blocked with 5% normal goat serum in PBS 0.1% triton and stained at 1:100 dilution of anti-SSEA1 antibody (DSHB, Hybridoma bank) in blocking buffer overnight. After washing the cells for 30 min with PBS 0.1% triton secondary antibody was added (Alexa Fluor 488, molecular probes) for 1 hour, cells counterstained with DAPI (Sigma), mounted with mounting medium (Histomount, electron microscopy sciences) and covered.

PGCs Transfection, Selection and FACS Sorting: Plasmid transfection of PGCs was done using lipofection or electroporation. For lipofection, Lipofectamine 2000 was used according to the manufacturer's protocol. 3-5×10⁵ cells were seeded in 96 well plate in AkoDMEM containing NEAA, pyruvate, vitamins, CaCl₂ and growth factors (activinA, hFGF and ovatransferin). 100 ng of plasmid, and 0.25 μl of Lipofectamine 2000 (invitrogen) were diluted separately in 20 μl of OPTI-MEM mix together, incubated for 20 minutes and pipetted on the cells. For electroporation, 3×10⁵ to 1.5×10⁶ cells were washed in AkoDMEM and electroporated at 1000V, 12 ms, 3 pulses on Neon electroporator (Invitrogen), and seeded immediately in 96 or 48 well plate, respectively, in antibiotics-free PGCs medium. Medium was changed after 1-3 hours. Selection with 25-100 g/ml G418 started 72 hours later for 2-4 weeks. Following selection, cells were individually isolated manually or by FACS sorting. For FACS sorting, gentle cells pipetting was done and cells were sorted in PGCs culture medium. Positive GFP cells sorted with FACS Aria II to new 96 wells plate, a single cell per well, or pooled. (FACS analysis performed using a BD FACS Aria II flow cytometer (BD, USA).

### Plasmid Preparation

Cloning of CRISPR Plasmids: CRISPR sequences were design using CRISPR design tool, Zhang lab, MIT. px330-GFP plasmid (modified from Addgene plasmid #42230) was cut using BbsI restriction enzyme and served us as the backbone for CRISPR site insertion to form the sgRNA. The oligos for the sgRNA CRISPR sites—CRISPR1, CRISPR3 (oligos P34-P35 and P36-P37, respectively) were denatured at 95 °C for 30 seconds, slowly annealed and ligated to the BbsI cut plasmids, transformed into E. coli, purified and sequence verified as described (Cong L, et al., Science. 2013 Jan. 3. 10.1126/science.1231143 PubMed 23287718).

Cloning ofpJet-HAs Plasmid: The genomic region downstream to the HINT1Z locus on the Z chromosome, containing both the 5′HA and 3′HA was amplified from PGCs DNA with P1 and P2 primers, using PCR (Kapa, Roche). PCR product was purified and ligated into pJet1.2 plasmid (Invitrogen) according to manufacturer protocol.

Construction of Targeting Vector: The pCAGG-IRES-Neo-GFP plasmid was used as template for PCR, using P5-P6 primers, to amplify the insert pCAGG-IRES-Neo-GFP. pJet-HAs plasmid was used as template for PCR, using P3-P4 primers, to amplify the vector containing 5′HA and 3′HA. Gibson assembly reaction was done to the purified vector and insert PCR products taking 0.03 μm, 0.06 μm linearized product, respectively. Gibson assembly reaction products were transformed to E. coli for plasmid preparation which was sequence verified.

Construction of the pCAGG-Optogene Vector: To generate the pCAGG-Optogene vector the optogenes plasmids pmCherry-CIBN-CreC and pmCherry-Cry2-CreN11 were used as a template to amplify the optogenes using the P40-P41 and P42-P43 primers, which yielded 1.3 kb and 2.1 kb products respectively. These two products share overlap sequences at the P2A site which was introduced in primers P41 and P42. Single-cycle overhang extension PCR was used to unite to two fragments, to a single 3.5 kb product which was cleaned from an agarose gel. This product was ligated to pJet1.2 shuttle vector that was used as a temple for PCR using primers P44 and P45, which contain tails with SmaI and NheI restriction sites, respectively. This product was digested using the appropriate restriction enzymes and was used as an insert for ligation to ligated to SmaI and NheI digested pCAGG-IRES-GFP plasmid that served as a vector. The ligation products were transformed to E. coi bacteria and the propagated plasmid was sequenced verified.

Construction of pGK-DTA-IRES-GFP Vector: To generate the pGK-DTA-IRES-GFP, the expression vector pSK BS-PGK-DTA was used as a template for PCR with primers P46 and P47 which contain extensions sequences for the XmaI and NheI restriction sites respectively. The 0.65 kb product was digested with the respective enzymes and was used as an insert for ligation to the XmaI-NheI complementary site in the pGK-IRES-GFP plasmid that served as a vector for the ligation. The ligation products were transformed to E coli bacteria and the propagated plasmid was sequenced verified.

In-ovo Electroporation: In-ovo electroporation was conducted essentially as was previously described. Fertile eggs were incubated for 56-60 h at 37.8 °C, the eggshell was windowed and plasmid DNA at a concentration of ~2 μg/μl was injected using a sharpened micro-pipette with an opening of 10-15 μm in diameter to the neural tube. Three pulses of 25 V, 30 ms were delivered using ECM 830 square wave electroporation system (BTX). Following electroporation, the eggshell was sealed with parafilm and the embryos were further incubated until analysis.

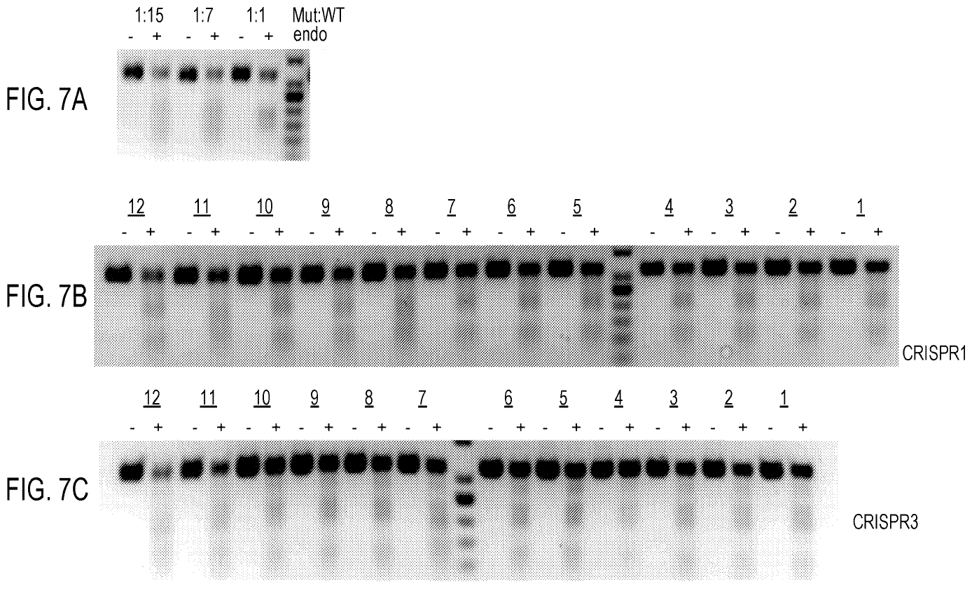

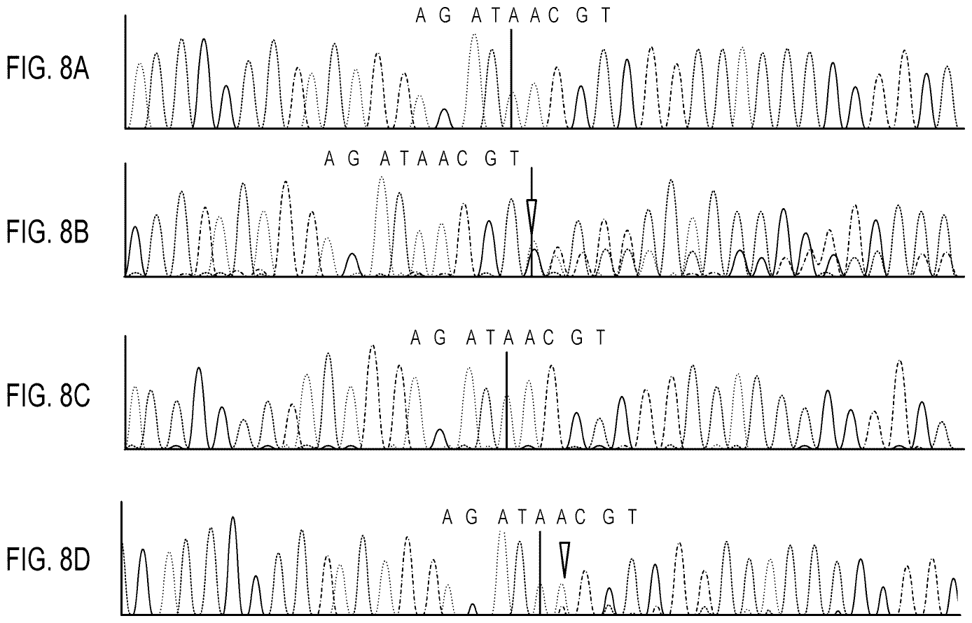

Endonuclease Assay: PGCs were transfected with CRISPR1 or CRISPR3 plasmids using Lipofectamine 2000 reagent. Forty-eight hours later, individual GFP positive cells were isolated into 96 well plate and grown to form pure colonies. DNA was collected and a 350 bp region flanking the CRISPR sites was PCR amplified with P38-P39 primers. The PCR products undergo denaturation at 95 °C and slowly annealed and incubated with T7 endonuclease for 1 h at 37 °C. For calibration purposes and as a positive control, the 350 bp PCR product was sub-cloned to pJet1.2 and the CRISPR site was mutated using site-directed mutagenesis. The mutation that was introduced replaced the WT sequence ATACCAGATAACGTgCCTTATTTGGCCGTT (SEQ ID NO:2) with ATACCAGATAACGTaatCCTTATTTGGCCGTT (SEQ ID NO:3). This artificial mutation served as a positive control to both the endonuclease assay (FIG. 7A) and for control sequencing (FIG. 8B).

Southern Blot Assay: Dig-labeling for the 5′HA, 3′HA and Neo gene probes were prepared by PCR amplification (Longamp, NEB) with primers P13-P14, P15-P16 and P11-P12, respectively, using DIG DNA labeling Mix (Roche). Fifteen g of genomic DNA were digested overnight at 37 °C with BglII restriction enzyme. DNA fragments were separated by electrophoresis on 0.8% (w/v) agarose gel (20 V, 12 h) and transferred onto positively charged nylon membranes (GE Healthcare). Following transfer, humid membranes were cross linked using a UV light set to 254 nm for 3 minutes on each side then rinsed with 2×SSC. Membranes were pre-hybridized for 2 hours at 42 °C using DIG Easy-Hyb hybridization solution (Roche). Probes (50 ng/ml) were denatured by heating to 95 °C for 5 minutes and immediately plunged into ice. Denatured probes were added to 10 ml warm DIG Easy-Hyb solution and hybridized for 12 hours at 42 °C. Membranes were washed twice for 10 minutes in 2×SSC, 0.1% SDS at room temperature under agitation and then washed 3 times for 30 minutes in 0.2×SSC, 0.1% SDS at 65 °C under agitation. Further washing and blocking was done with a DIG wash and block buffer set (Roche) and according to their protocol. DIG labeling was detected using Anti-Digoxigenin-AP antibody 1:10000 (Roche) followed by chemiluminescence reaction using CDP-Star reagent (Roche). Images were taken using G:BOX gel imaging system (Syngene).

PGC Injection To Embryos And Whole Mount Staining: Freshly laid eggs were incubated with the pointed end up for 58-62 hours at 37.8 °C with 55% humidity. Following incubation, 4-8 mm window was opened in the egg shell and 3000-8000 PGCs were injected to the blood stream using sharpened micropipette with opening of ~30-40 μm. The window was covered with white egg membrane and further sealed with a Parafilm (Parafilm) or a Leukoplast (BSN medical GmbH) tape. Embryos were incubated until hatching. Some gonads of injected embryos were isolated and taken for whole-mount GFP staining. Gonads were fixed in 4% PFA, washed for 2 h with PBS blocked with 5% normal donkey serum in PBS 1% Triton and stained at 1:20 dilution of mouse anti-SSEA1 antibody or rabbit anti GFP antibody 1:500 (Abcam) in blocking buffer overnight. After washing for 2 h with PBS 1% triton, a secondary donkey anti mouse cy3 antibody 1:500 was added (Jackson Immunoresearch laboratories) or secondary alexa488 anti rabbit antibody 1:500 (Molecular Probes) for 3 hours in blocking buffer. Tissue counterstained with DAPI (Sigma) and mounted in glycerol, and imaged by confocal microscope (Leica, TCS SPE, Wetzlar, Germany).

The sequences for primers P1 to P32 are listed in SEQ ID NOs:4-35, the sequences for primers P34 to P47 are listed in SEQ ID NOs:36-49.

The sequences for the plasmids are listed below:

1. pX330-GFP (SEQ ID NO: 50); 2. CRISPR1 (SEQ ID NO: 51); 3. CRISPR3 (SEQ ID NO: 52); 4. pJet-Has (SEQ ID NO: 53); 5. pCAGG-Neo-IRES-GFP (SEQ ID NO: 54); 6. Targeting Vector (SEQ ID NO: 55); 7. pmCherry-Cry2-CreN (SEQ ID NO: 56); 8. pmCherry-CIBN-CreC (SEQ ID NO: 57); 9. pB-RAGE-GFP (SEQ ID NO: 58); 10. pCAGG-IRES-GFP (SEQ ID NO: 59); 11. pCAGG-Optogenes (SEQ ID NO: 60); 12. pB-RAGE-mCherry (SEQ ID NO: 61); 13. pSK BS-PGK-DTA (SEQ ID NO: 62); 14. pGK-IRES-GFP (SEQ ID NO: 63); 15. pGK-DTA-IRES-GFP (SEQ ID NO: 64).

### Results

### PGC Lines Derivation And Characterization

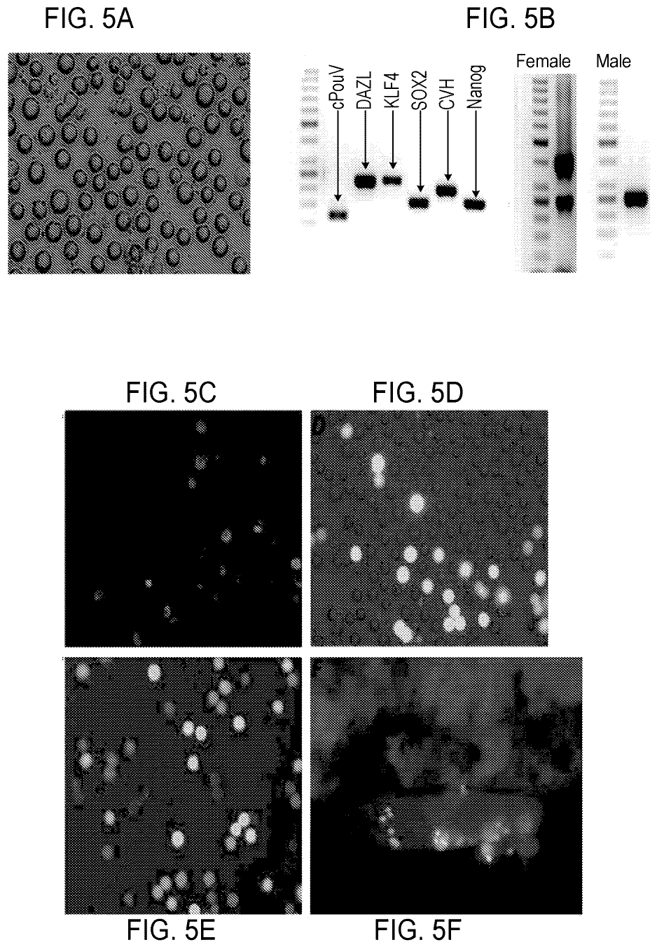

During the earliest stages of embryonic development, soon after oviposition and prior to the initiation of gastrulation, PGCs migrate rostrally to the Germinal Crescent region at the anterior part of the extra-embryonic mesoderm layer. It is thought that this migration “protects” the PGCs from undergoing differentiation processes as somatic cells do. It is not until the formation of the Area Opaca Vasculosa, blood and heart-beat, after about 2.5 days of incubation (Stage 14-17 H&H), that the cells return to the embryo through the blood stream and colonize the Genital Ridge which will give rise to the gonads. At these stages, using a micropipette with a ~40-60 μm diameter opening, 1-3 μl of blood was collected from the vasculature system of the embryos and transferred into a well containing PGC culture medium in a 48 well plate. The PGC culture medium allows for fast division of the PGCs (20-24 hours of cell cycle) while retaining their undifferentiated state under feeder free conditions. After 2-3 weeks in culture, blood cells degraded and disappeared. Within another 1-2 more week, the cultured PGCs became confluent (FIG. 5A). These cells can be further grown for gene modification or can be successfully frozen and thawed for latter modifications. Chicken PGCs in culture have been extensively characterized in the literature using morphological features, protein and mRNA expression patterns and finally by their ability of gonad migration when injected back into the vasculature of a stage-matched recipient embryo. These characteristics were examined in the produced PGC cell cultures to show that they keep the well-established PGC features. Morphologically, the PGCs are big, slightly granulated cells about 15-20 m diameter containing large nuclei. The PGCs are totipotent cells, thus they express pluripotent markers such as the cPouV, SOX2, KLF4 and Nanog and two unique germ cells markers—cVH and DAZL. For each PGC line, DNA was extracted for sex determination using primers for the Ribosomal S18 (P19-P20, 256 bp product size) as a positive control and primers for the W chromosome (P17-P18, 415 bp product size) to identify females (FIG. 5B). Additionally, the PGCs express the membrane SSEA-1 antigen4 (FIG. 5C).

Ten lines of PGCs were established from layers and broilers, both male and female lines. Plasmid transfection was carried out using cationic-lipid transfection reagent Lipofectamine 2000 which interacts with the negatively charged DNA, allowing its penetration into the cell. Transfecting with GFP encoding plasmid (pCAGG-GFP) resulted in about 15-20% transfection efficiency (FIG. 5D). Further, transfection of PGCs using electroporation resulted in higher efficiency of up to 90% (FIG. 5E). To demonstrate that cultured PGCs successfully colonize the gonads, GFP-expressing PGCs were injected into the blood stream of stage 14-16 H&H and the embryos were incubated for 10 days. The embryos were dissected, and GFP-positive cells were identified in the gonads (FIG. 5F).

### Designing the CRISPR-Cas9 Targets On The Z Chromosome

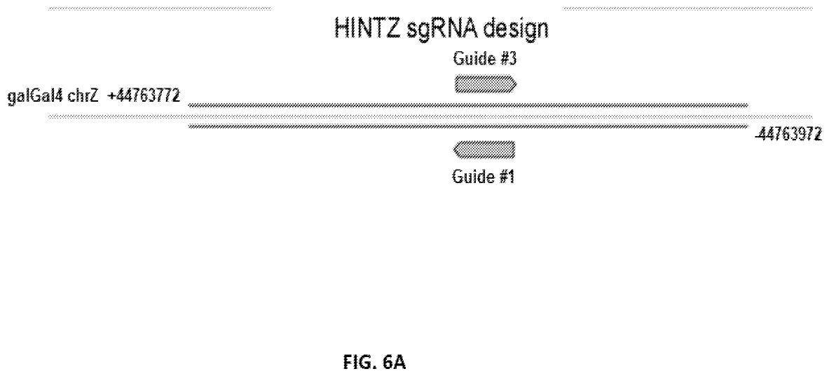

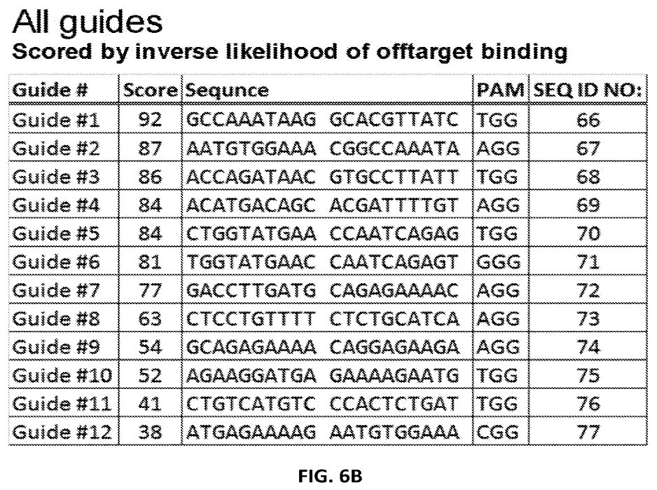

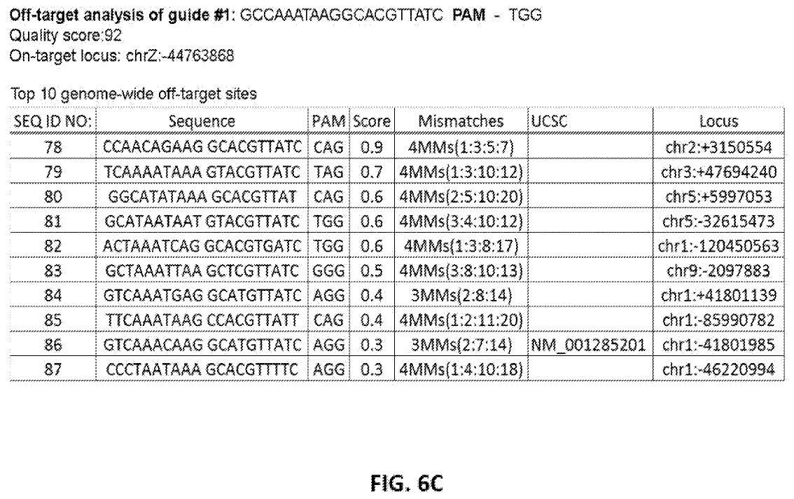

In one embodiment, DNA editing into the Z chromosome were done using CRISPR-Cas9 and homologous recombination processes. While CRISPR-Cas9 system will directly cut the DNA at a specific site of the Z chromosome, the endogenous repair system using homology recombination process will allow targeted insertion of the desired DNA into the precise location. For this purpose, constructing a targeting vector plasmid which contains the homology arms corresponding to the insertion site on the Z chromosome is required. The site for DNA insertion at the Z chromosome downstream the coding gene HINT1Z was chosen. The use of the CRISPR system has been shown in many studies to improve direct DNA insertion events. Extensively used for that purpose, the px330 plasmid includes the sgRNA site and the Cas9 enzyme. The sgRNA site contains a unique sequence which directs the Cas9 enzyme to the target site and leads to specific genome targeted DSDB. Using a CRISPR design engine tool, a unique sequence for the sgRNA was identified as shown in FIG. 6A. The top 12 guides, according to their score are depicted in FIG. 6B. The sequences for guides 1 to 12 are listed in SEQ ID NOs:66-77.

Guides #1 and #3 were chosen by conventional similarities of the secondary structure and were used to check possible off-target sites in the chicken genome which scored by the mismatch extent. The top 10 results of search for potential off-targets for guide #1 are shown in FIG. 6C and SEQ ID NOs:78-87. Notably, the top 6 off-targets have 4 mismatches, highlighting the specificity of this guide.

DNA sequence insertion was carried out by cutting a modified px330 plasmid, which contains in-frame GFP fused to the c-terminus of Cas-9. Annealed primers containing the sgRNA sequences were ligated to the BbsI restriction enzyme as previously described. (FIG. 6B). Ligation products were transformed to E. coli, plasmids were purified and sgRNA insertions were verified by sequencing.

### Activity Validation of The CRISPR-Cas9 System

By growing PGCs in feeder free culture medium, pure colonies originating from single cells were obtained, thereby allowing characterization of the efficiency of the CRISPR-Cas9 system. To this end PGCs were transfected with either pX330-GFP-CRISPR1 and pX330-GFP CRISPR3 plasmids, and clonal colonies were grown. Total genomic DNA was extracted from colonies originating from single cells expressing GFP. The DNA was analyzed by endonuclease assay and sequenced. For the endonuclease assay, a positive control was designed. This control was a 320 bp PCR product with inserted mutations at the predicted site for CRISPR-Cas9 activity. This product was mixed with similar length WT product in different ratios, 1:15, 1:7, 1:1-mutated:WT respectively, and the annealed mixture was subjected to endonuclease activity (FIG. 7A). Two short bands at the predicted size of 136 bp and 184 bp were clearly visible at the ratios of 1:7 and 1:1, indicating the assay was properly working. Similarly, the same assay was performed on genomic DNA obtained from 12 colonies, transfected with either CRISPR1 and CRISPR3 plasmids (FIGS. 7B, 7C). In 9 out of the 12 colonies, a clear doublet at the predicted size was observed. This indicates that both CRISPR1 and CRISPR3 plasmids, efficiently generate DSDBs at the predicted site.

For sequencing analysis, PCR products that were used for endonuclease assay (FIGS. 7A-7C), were also sequenced (FIGS. 8A-8D). Sequencing of the WT negative control revealed the predicted cleavage site of CRISPR1 (FIG. 8A). Sequencing a mixture of WT and artificially mutated product, as a positive control, revealed the appearance of double peaks on the DNA chromatogram, immediately after the predicted cleavage site (arrowhead, FIG. 8B). Similar sequencing of the same genomic region in the transfected colonies revealed both negative (FIG. 8C) and positive (arrowhead, FIG. 8D) colonies, whereas the latter were >70% of the cases.

### Constructing Targeting Vector For Genome Integration

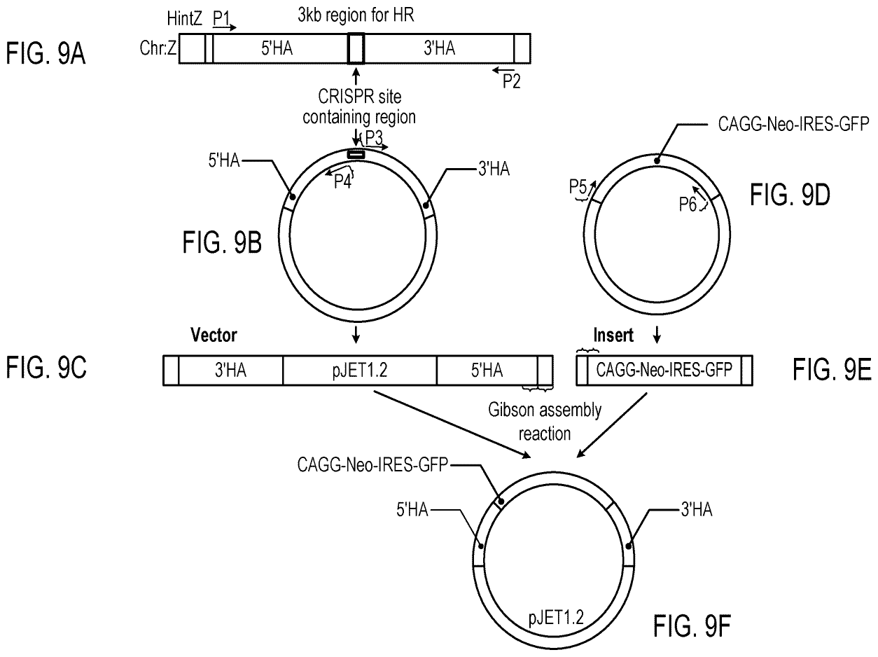

To demonstrate a targeted genomic integration to the Z chromosome, using HR, a targeting vector was designed (FIGS. 9A-9F). The vector contains a pCAGG promoter followed by the neomycin selections gene, internal ribosome entry site (IRES), GFP and the Rabbit beta-globin polyadenylation site. This cassette was flanked by ~1.5 kb homology arms at the 5′ and 3′ ends respectively. To generate this vector, a ~3 kb DNA fragment, containing both homology arms was amplified using primers P1 and P2, and ligated to a shuttle vector pjet1.2. Full sequencing of this fragment was found to be identical to the chicken genome sequence. This plasmid—pJet-HAs, was used as a template to generate a linearized PCR product containing two separated homology arms excluding a 23 bp sequence between them, which contains the CRISPR sgRNA sites. The amplification was done using the P3 and P4 primers which contain sequences, at their 5′ end, which correspond to the edges of the pCAGG-Neo-IRES-GFP cassette. This linear PCR product is referred to as the “vector”. The pCAGG-Neo-IRES-GFP plasmid was used as a template to generate a linear PCR product. This fragment was amplified using primers P5 and P6 containing sequences which correspond to the 3′ and 5′ ends of the 5′HA and 3′HA ends, respectively. This product is referred to as the “insert”. The vector and the insert were stitched together using the Gibson assembly reaction to create the final targeting vector.

### Homologous Recombination To The Z Chromosome Using The Targeting Vector And CRISPR Plasmids

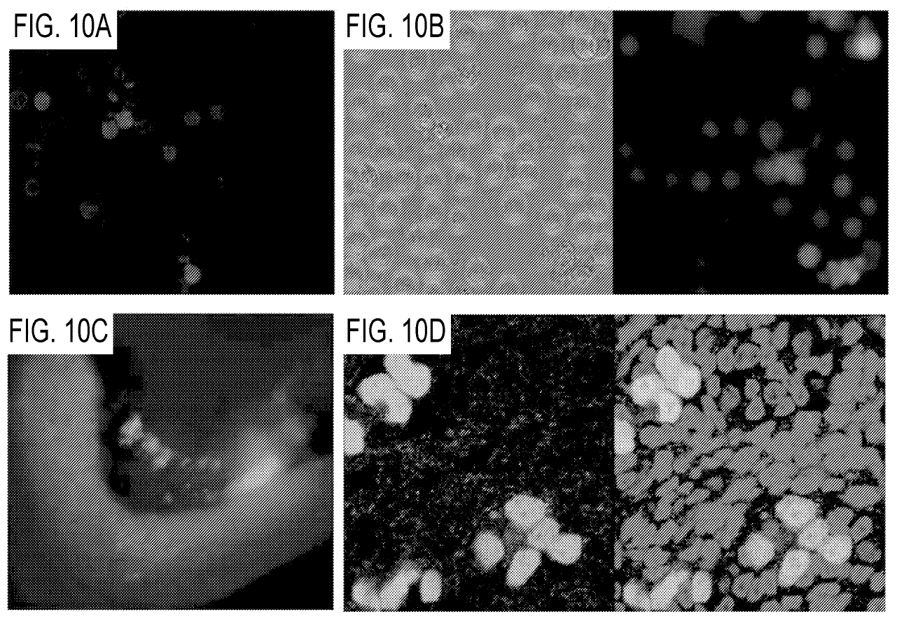

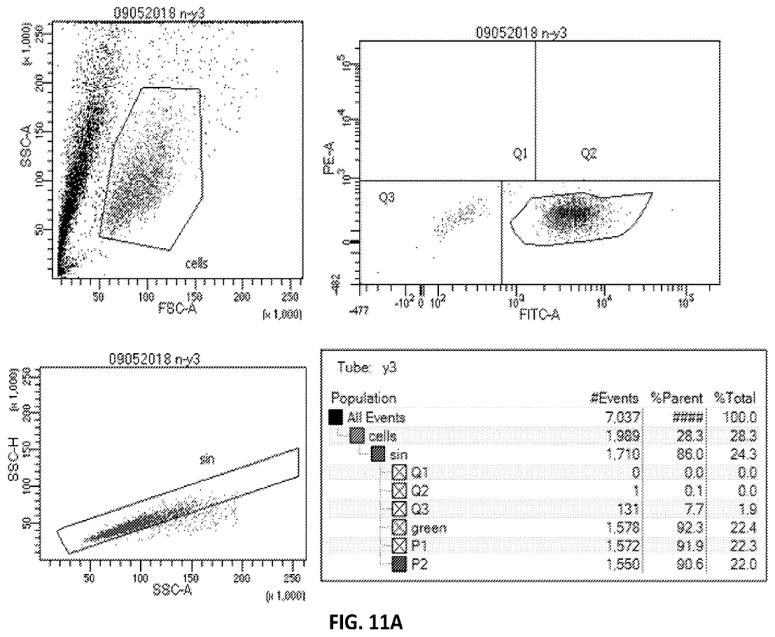

The ability to obtain pure PGCs colonies from a single cell enables the identification of positive colonies that underwent correctly inserted HR, using methods such as PCR and Southern blot. For PGC transfection, lipofection with a 5-10% (FIG. 10A) transfection efficiency or electroporation with >40% efficiency were used. Transfection was carried out with two plasmids, the targeting vector and one of the two CRISPR plasmids described above (CRISPR1 or CRISPR3). Following the transfection, the cells were left to recover for 24 hours, and transferred to G-418 containing media for selection. After two weeks of selection, only G-418 resistant cells survived, of them >99% were GFP positive (FIG. 10B). To verify that the cells retain their ability to colonize the gonads, they were injected into host embryos as was described above in FIG. 1F (FIG. 10C). The gonads were immuno-stained with anti-GFP antibody and the colonization of GFP-positive PGCs cells in the gonads was verified using confocal microscope (FIG. 10D).

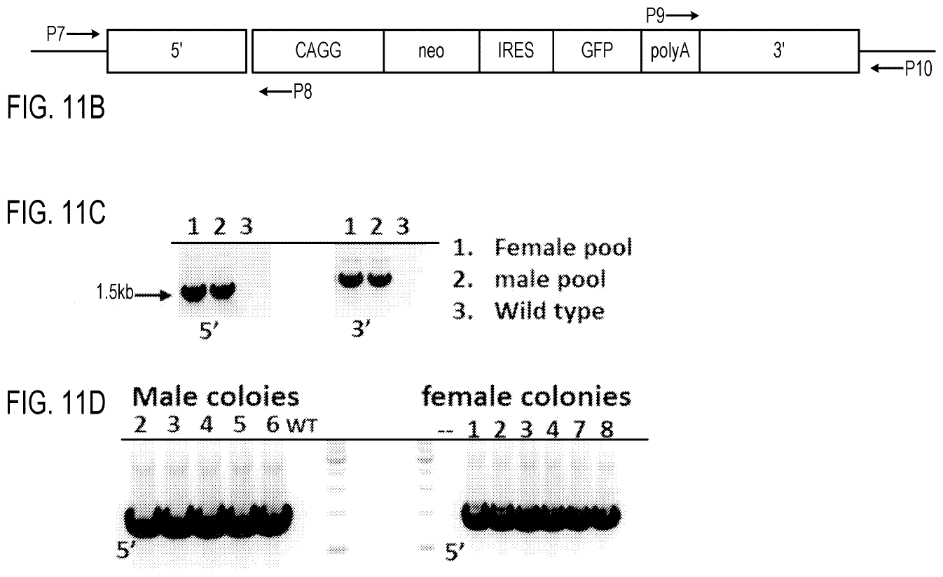

The G-418 resistant, GFP-positive cells consist of a potentially heterogeneous population. Thus, in order to verify the HR integration, and to obtain pure homogenous population, single GFP-positive cells were separated using FACS sorting to 96 well plate (FIG. 11A). Pure colonies were raised and genomic DNA was extracted for PCR and Southern blot analysis. In parallel, pooled GFP-positive cells were FACS sorted. For PCR analysis, two sets of primers were designed. The first, forward P7 upstream to the 5′HA and reverse P8 from the CAGG promoter (1.6 kb product size), and the second, forward P9 from the rabbit beta-globin polyadenylation site and reverse P10 downstream to the 3′HA (1.8 kb product length, FIG. 11B). Both in pooled cells (FIG. 11C) and in pure colonies (FIG. 11D), the expected products for the 5′ and 3′ were detected, indicating that correct HR integration had occur in these cells.

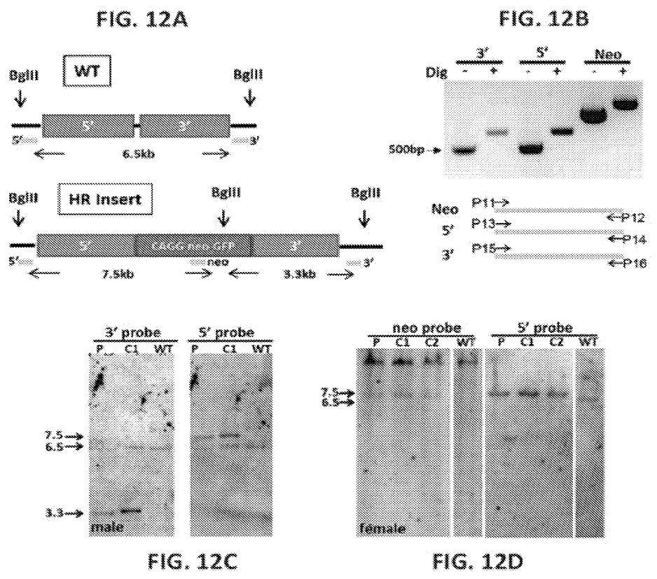

To further verify the correct HR integration as well as to confirm that only a single copy of the targeting vector was integrated into the genome, a Southern blot analysis was conducted. Two PGCs cell lines from male and female donors were analyzed. Notably, the female line has only a single copy of the Z chromosome. Three digoxigenin-labeled (dig-labeled) DNA probes were designed (see FIG. 12A and FIG. 12B). The first two probes, amplified using primers P11-P12 and P13-P14, 500 bp long each, are located upstream and downstream to the 5′ and 3′ HAs respectively. The third probe, amplified using primers P15-P16, 704 bp long, is designed to detect the Neo gene inside the targeting vector, thus it allows for confirmation that only a single copy of the vector was integrated. The BglII restriction enzyme was used to cleave the genomic DNA for analysis. Two restriction sites, ~6.5 kb apart from each other, are located on the WT chromosome, upstream and downstream to the 5′ and 3′ probes respectively. Additional BglII site is located in targeting vector, yielding a predicted 7.5 kb and 3.3 kb fragments to identify correct HR integration. The results of the Southern blot analysis on the genomic DNA extracted from the male PGCs line revealed 2 bands at the predicted size, 6.5 kb for the WT allele and 7.5 kb and 3.3 kb for the allele which underwent correct HR integration, for the 5′ and 3′ sites, respectively. This was confirmed for both the DNA from pooled cells as well as for pure colonies (FIG. 12C). A similar analysis was carried out for the female PGC cell line. In this case a single band at the predicted size of 7.5 kb for the 5′ integration site was found. As the female genome contains only a single copy of the Z chromosome, no WT allele (6.5 kb) was detected. Probing the Neo gene, revealed a single band at the predicted size of 7.5 kb, confirming that only a single copy of the targeting vector was integrated into the genome (FIG. 12D).

### Validation of The Optogenetic System In HEK293 Cells In-Vitro And In Chicken Embryos In-ovo.

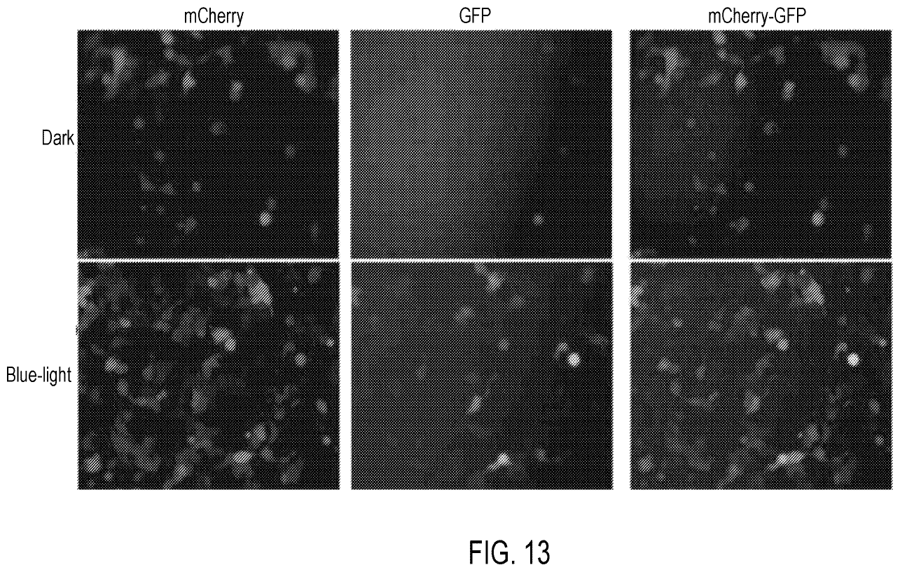

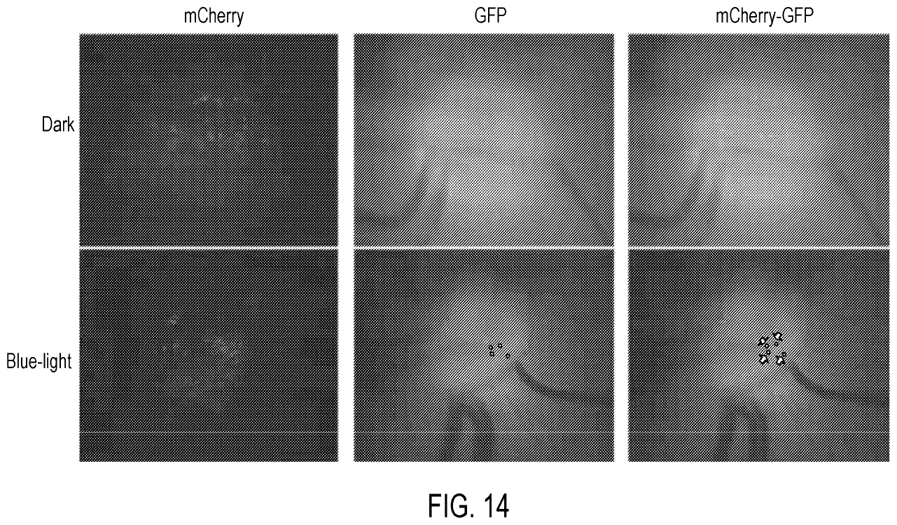

To verify the activity of the inducible system in-vitro and in chicken embryos in-ovo, three plasmids: pmCherry-Cry2-CreN, pmCherry-CIBN-CreC and the reporter PB-RAGE-GFP were transfected to HEK293 cells (FIG. 13) and to chick embryos (FIG. 14). The first two optogenetic plasmids encode the reporter gene mCherry which confirms successful transfection. The PB-RAGE-GFP epression vector contains a multiple stop codons sequence flanked by LoxP sites upstream to the GFP coding region. Upon Cre activation, the STOP codons are removed thus allowing the GFP to be expressed. While in negative-control HEK293 cells that were triple-transfected and kept in the dark, there were no GFP-positive cells (FIG. 13, upper row). In cells that were exposed to blue-light illumination, 24 hours after transfection, many cells expressed GFP (FIG. 13, lower row), confirming the activation of the optogenetic system in these cells.

To verify the activity of the optogenetic system in-ovo, a triple transfection with pmCherry-Cry2-CreN, pmCherry-CIBN-CreC and PB-RAGE-GFP plasmids by electroporation into chicken embryo neural tubes at stage 16 H&H was performed. Twelve hours following electroporation, experimental group embryos were subjected to 15 seconds of blue-light illumination, while negative control embryos were kept in the dark. The embryos were incubated for an additional 12 hours and checked for GFP expression under fluorescent stereoscope (FIG. 14). While in embryos that were kept in dark (FIG. 14, upper row), only mCherry was expressed, thus confirming successful electroporation, in embryos of the experimental group, GFP positive cells were clearly evident (FIG. 9, lower row), confirming that the light-inducible Cre was activated.

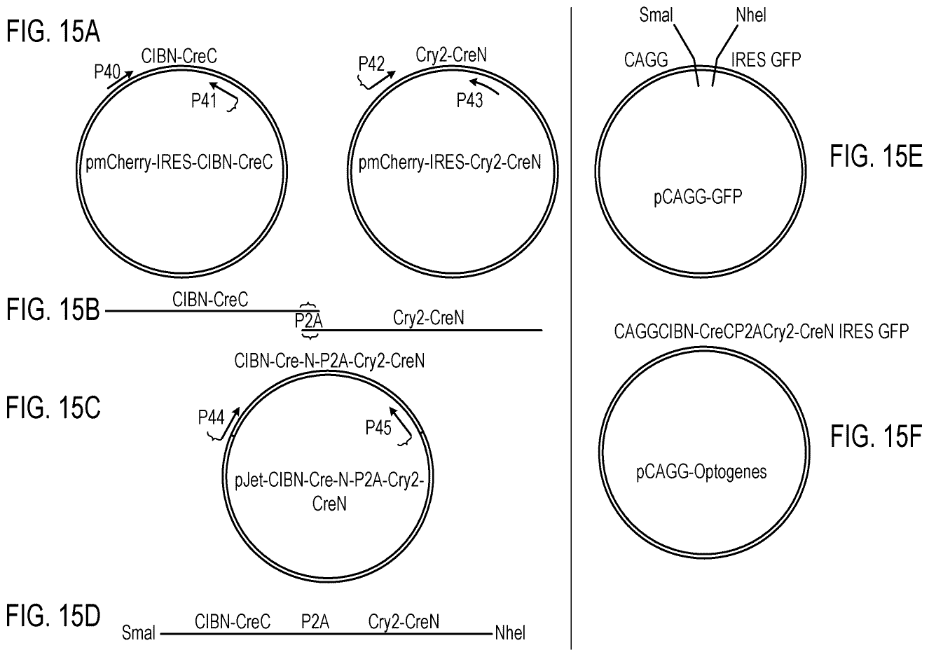

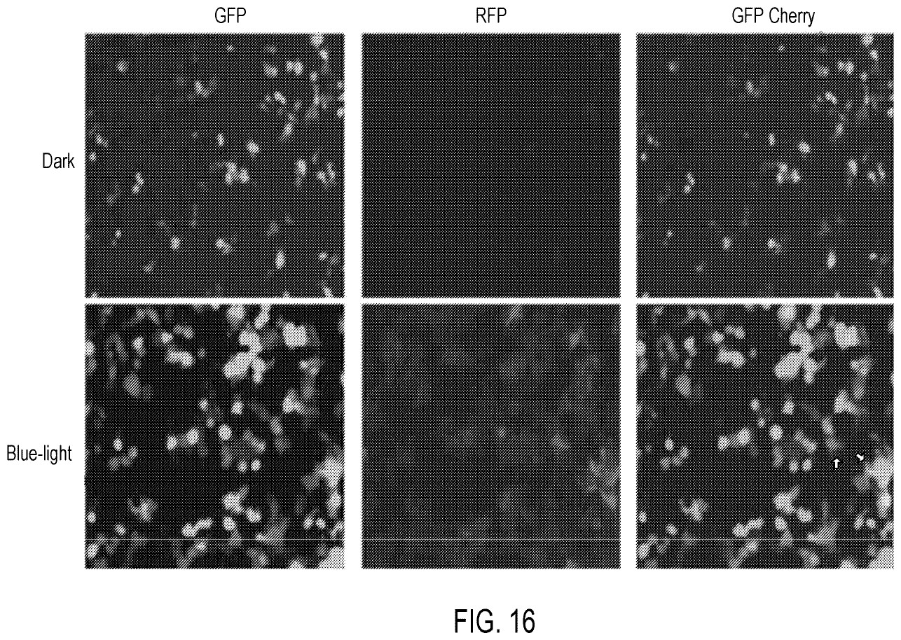

The optogene plasmids pmCherry-Cry2-CreN and pmCherry-CIBN-CreC drive the expression of the genes using the CMV promoter which is unfavorable in chicken cells. To overcome this and to combine the two to a single vector, a plasmid vector which drives the expression of CIBN-CreC and Cry2-CreN, linked by the P2A self-cleaving peptide, followed by IREG-GFP, under the CAGG promoter, was designed, which is highly active in chicken cells. Synthesis of pCAGG-CIBN-CreC-P2A-Cry2-CreN-IRES-GFP was based on a modification of the original optogenes plasmids described in Kennedy et al., 2010 (Nat Methods. 2010 December; 7(12): 973-975). Each of these plasmids encode mCherry followed by IRES sequence with either CIBN-CreC (a truncated form of CIB1 fused to the C-terminal of the Cre enzyme) or CRY2-CreN (Cryptochrome 2 fused to the N-terminal of the Cre enzyme. FIG. 15A). The goal of the following cloning was to join the two fusion optogenes with self-cleaving peptide P2A, under the CAGG promoter, followed by IRES-GFP. To this end, the CIBN-CreC plasmid was used as a template for PCR with P40 and P41 primers and the CRY2-CreN plasmid was used as a template for PCR with P42 and P43 primers (FIG. 15A). Notably primers P41 and P42, which contain the P2A cleavage site, share overlap sequence that allows the two products to be merge by a single-cycle overhang extension PCR (FIG. 15B). This product which contains CIBN-CreC-P2A-CRY2-CreN was ligated to a shuttle vector pJet1.2, and was sequences verified (FIG. 15C). This plasmid served as template for PCR with primers P44 and P45 which added to the product the SmaI and NheI restriction site on the 5′ and 3′ ends, respectively (FIG. 15D). This product was digested with the restriction enzymes and ligated to the pCAGG-TRES-GFP plasmid which was also cut using the same enzymes (FIG. 15E). This ligation product contains the CAGG promoter, followed by CIBN-CreC, P2A self-cleaving peptide, Cry2-CreN, TRES, GFP and the rabbit beta-globin poly-adenylation site (referred to herein as pCAGG-Optogenes), and was sequenced verified (FIG. 15F).

To verify the activity of the pCAGG-Optogene vector in-vitro, the plasmid, which expresses GFP as a reporter for successful transfection, was co-transfected into HEK293 cells with pB-RAGE-mCherry. Like the the PB-RAGE-GFP vector described above (FIG. 13), the pB-RAGE-mCherry contains a multiple stop codon sequence flanked by LoxP sites upstream to the mCherry coding region. Upon Cre activation, the stop codons are removed thus allowing the mCherry to be expressed (FIG. 16). While in HEK293 cells that were co-transfected and kept in the dark there were no mCherry-positive cells (FIG. 16, upper row). In contrast, cells that were exposed to blue-light illumination, many cells were expressing mCherry (FIG. 16, lower row), confirming that the single-vector strategy of the pCAGG-Optogenes, preserves the optogenetic properties of the system.

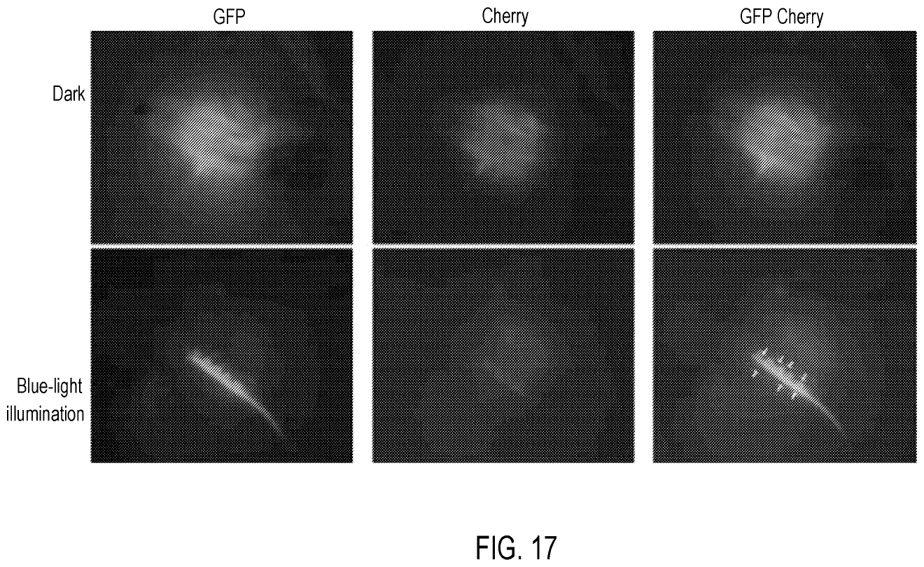

To verify the activity of the pCAGG-Optogenes vector in living chick embryos in-ovo, the plasmid was co-transfected by electroporation to stage 14-16 H&H chick embryos together with pB-RAGE-mCherry. Twelve hours following electroporation, negative-control group eggs were kept in the dark, while experimental group embryos were exposed to blue-light for 15 seconds (FIG. 17). Both groups were further incubated for 12 h and examined under a fluorecent stereoscope. Following incubation, both groups revealed high level of GFP expression, indicating the successful electroporation. However, only in the light-exposed group (FIG. 17, lower row), mCherry-positive expressing cells were identified, indicating that the optogenetic system, using the single-vector strategy of the pCAGG-Optogenes, was activated in a light-inducible manner.

### Inducing Lethality In Chick Embryos

To demonstrate the feasibility of causing mortality using a toxin, the coding region of the DTA12, commonly used as negative selection marker, was cloned into an expression vector containing the pGK promoter followed by an IRES GFP (pGK-IRES-GFP). This plasmid also served as a negative control. The DTA coding region was cloned upstream to the IRES sequence giving rise to the pGK-DTA-TRES-GFP, which upon expression in cells inhibits protein synthesis which leads to cellular death.

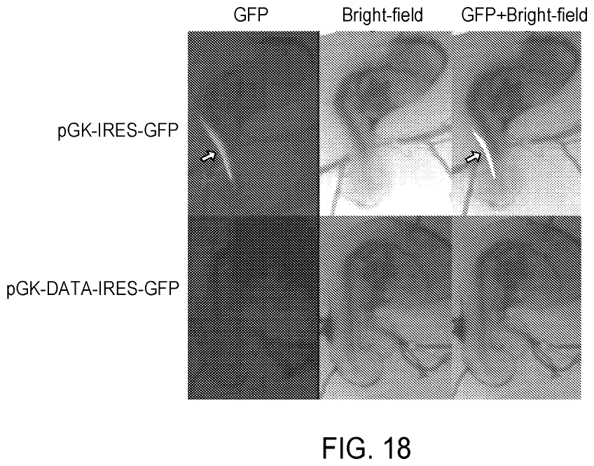

To test the effects of DTA expression in chicken embryos, stage 14-16 H&H embryos were electroporated with either pGK-IRES-GFP, as a negative control or with pGK-DTA-IRES-GFP vector. Twelve hours following electroporation, the embryos were analysed for the expression of GFP under a fluorescent microscope (FIG. 18). While in control embryos, GFP was widely expressed in the neural tube (FIG. 18), in DTA expressing embryos, no GFP expression was detected, indicating that protein synthesis was blocked in these cells.

### Example 2

The aim of the experiments described herein was to introduce a lethality-inducing cassette into to the Z chromosome of either male or female-derived PGCs. The final goal was to obtain hens harboring a genomic insertion on the Z chromosome. This chromosome will segregate only to the next generation male embryos that upon blue-light induction will activate a lethality-inducing cassette, thus the male embryos will die at early stages of embryogenesis.

The cassette AKA the targeting vector (TV) has 3 elements as described herein. The first element is the “Homology arms”. The second element is the “Optogenetic inducible element”. The third element is the “lethality-inducing cassette”. In one embodiment, two ~1.5 kb homology arms are located on both 5′ and 3′ ends of the TV. This is designed to direct the homologous recombination (HR) downstream to the HINT1Z locus located on the Z chromosome. This site for HR was chosen since the HINT1Z gene is transcribed in PGCs as well as in whole blastoderms (these are freshly laid chick embryos). All other openly transcribed regions on the Z chromosome are also potentially good candidates for this purpose.

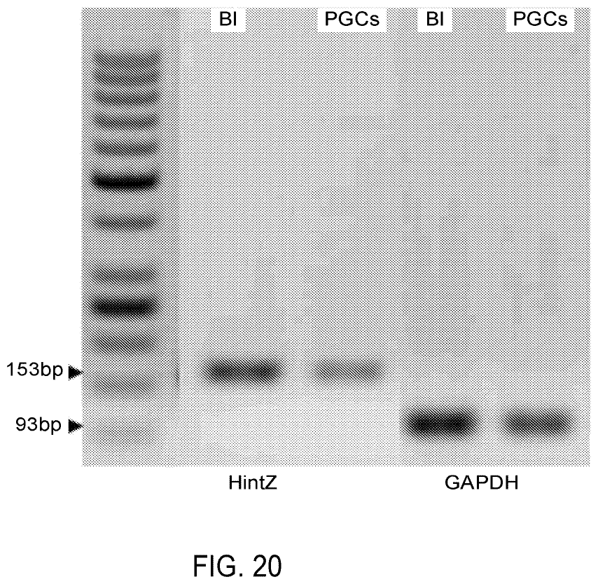

FIG. 20 shows the RT-PCR using FWD primer (SEQ ID NO: 88) and REV primer (SEQ ID NO: 89) on cDNA from PGCs and whole blastoderms. Product predicted size 153 bp.

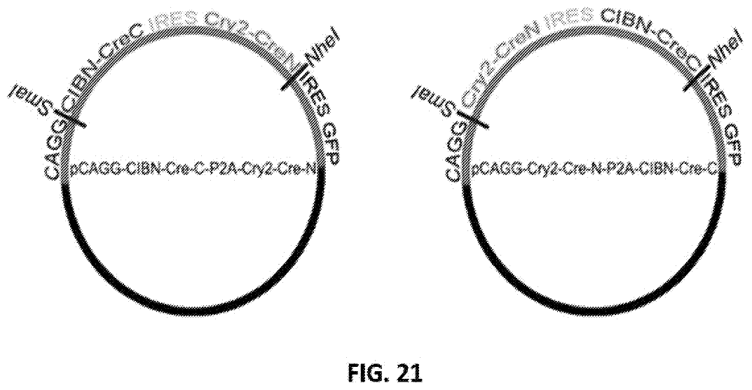

The second element is the “Optogenetic inducible element”. In one embodiment, the optogenetic systems are based on proteins that under certain light wavelengths are excited and change conformation that allows them to be dimerized. It is possible to fuse these proteins with additional proteins so that there will be transcription of a target gene only when the two-optogenetic proteins dimerize. For example, if the first protein is fused with a DNA targeting domain, such as Gal4-BD (Gal4 binding domain), and the second protein is fused with a transcription activator, such as Gal4-AD (Gal4 activation domain), it is possible to activate downstream genes. Similarly, a repression domain could be used to repress gene expression. However, an issue with optogenes is that they have a certain amount of basal dimerization and therefore gene activation that happens regardless of light induction. Finding the right balance between sensitivity to induction and basal dimerization is key to their use. A modified version of the system developed in Kennedy, M. J. et al. (Rapid blue-light mediated induction of protein interactions in living cells. Nat. Methods 7, 973-975, 2010) was used. The original optogene plasmids are pmCherry-Cry2-CreN and pmCherry-CIBN-CreC that drive gene expression using the CMV promoter, which is unfavorable in chicken cells. To overcome this and to combine the two to a single vector, a plasmid vector was designed to use the CAGG promoter, which is highly active in chicken cells, to drive the expression of CIBN-CreC and Cry2-CreN that are linked by the IRES sequence and followed by IREG-GFP. Synthesis of pCAGG-CIBN-CreC-IRES-Cry2-CreN-IRES-GFP was based on a modification of the original optogenes plasmids described in Kennedy et al., 2010 (Nat Methods. 2010, 7(12): 973-975). The goal of cloning was to join the two fusion optogenes with IRES, under the CAGG promoter, followed by IRES-GFP. Construction of similar plasmid in which the order of the optogenes, namely Cry2-CreN followed by CIBN-CreC, was also performed successfully and yielded similar results. FIG. 21 shows a schematic representation of the above described plasmids.

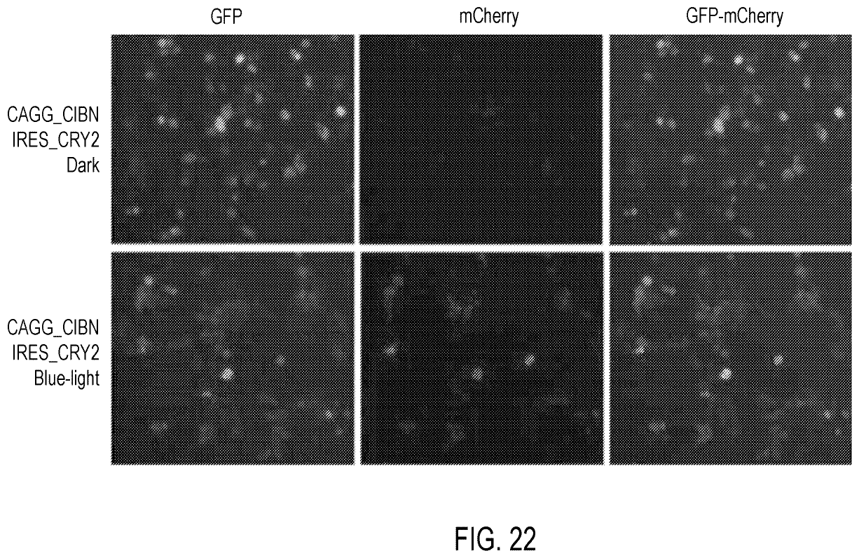

To verify the activity of the pCAGG-Optogene vector in-vitro, the plasmid that expresses GFP as a reporter for successful transfection was co-transfected into HEK293 cells with a reporter plasmid pB-RAGE-mCherry. This plasmid contains Lox-STOP-Lox (LSL, RAGE) element between the pCAGG promoter and the mCherry reporter gene. Thus, when co-transfected with the optogene plasmid, upon blue-light illumination and Cre activation, the LSL is removed and the mCherry is expressed. In the dark when the system is inactive, only the reporter GFP from the optogene plasmid is expressed, as shown in FIG. 22.

To validate the activity of the optogenetic system in living chick embryos in-ovo, the optogenetic plasmid and the reporter pB-RAGE-mCherry plasmid were co-electroporated to the neural tube of 56-60 h incubated chick embryos. The plasmid DNA mixture was injected to the lumen of the neural tube using a sharpened micropipette and electroporation was applied using 2 tungsten electrodes, 3-5 mm apart (Electroporator BTX830). Four pulses of 30V, 45 ms each were delivered with 745 ms interval between each pulse. Following electroporation the eggs were sealed and incubated for 12-18 h. Then, while eggs in control group were kept in dark, eggs in experimental group were exposed to 1 min of blue light illumination through the eggshell. Both groups were placed back in the incubator for another 12-18 hours before analysis.

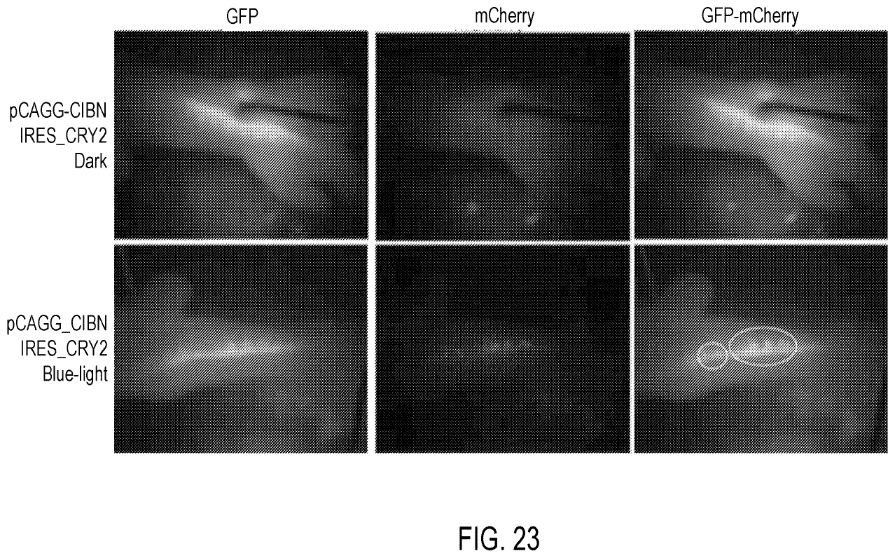

As shown in FIG. 23, in all experimental group, the reporter GFP was highly and widely expressed in all embryos, indicating a successful electroporation and expression of the plasmids. There was no mCherry expression in eggs that were kept in the dark. In contrast, mCherry was expressed in the neural tube in eggs illuminated by blue-light.

An improved system of the CIBN-Cry2 can be made by modifying the Cry2. This modification includes an insertion of L348F mutation in a truncated Cry2 gene which contains only the first 535 amino acids—Cry2-A535-L348F, as published in Nature Chemical Biology volume12, pages425-430 (2016).

As an alternative approach to the CIBN-Cry2 optogenetic system, another conceptually similar system can be used to achieve the same goals. This system is based on the MAGNET system, in which two optogenetic proteins—positively charged β-Mag and negatively charged N-Mag dimerize upon blue light illumination. Each of these proteins is fused to inactive part of the Cre recombinase enzyme and upon dimerization of the two optogenes, an active form of Cre is created (Nature Chemical Biology volume12, 1059-1064 2016).

The third element is the “lethality-inducing cassette”. In one embodiment, the lethality-inducing cassette comprises a gene (e.g. a toxin) which drives early embryonic death by means of cell death or severe intervention in molecular signaling pathway that is required for early stages of embryogenesis (e.g. BMP4). For example, this can be achieved by inducing programmed cell death (apoptosis) with toxins such as the Diphtheria toxin A (DTA), by expressing Caspase genes such as Caspase3 or a mutated constitutively-active mutated form of Caspase 3, or by expressing the inhibitor protein of BMP4—Noggin. The nucleotide sequence for DTA is set forth in SEQ ID NO: 92. The amino acid sequence of DTA is set forth in SEQ ID NO: 93.

In one embodiment, the nucleotide sequence for Chicken Casp3 CDS (WT) is set forth in SEQ ID NO: 94. The amino acid sequence for Chicken Casp3 CDS (WT) is set forth in SEQ ID NO: 95.

In one embodiment, the nucleotide sequence for constitutively-active (mutated) form of Chicken Casp3 is set forth in SEQ ID NO: 96. The amino acid sequence for constitutively-active (mutated) form of Chicken Casp3 protein is set forth in SEQ ID NO: 97.

In one embodiment, the nucleotide sequence for Noggin is set forth in SEQ ID NO: 98. The amino acid sequence for Noggin is set forth in SEQ ID NO: 99.

In certain embodiments, the “lethality-inducing cassette” is based on a gene that disrupts an essential signaling pathway. The sooner death is induced, the earlier the embryo will die. In certain embodiments, the essential signaling pathway involves a bone morphogenetic protein (BMP). In certain embodiments, the BMP is BMP4. In certain embodiments, the lethality-promoting protein is an antagonist of BMP4. In certain embodiments, the BMP4 antagonist is Noggin. In certain embodiments, the expression of the lethality-promoting protein Noggin is induced at a developmental stage in which the BMP pathway is active and required. In certain embodiments, the BMP pathway is active or induced during blastulation, gastrulation, neurulation, or organogenesis. In certain embodiments, the optogenetic system is activated soon after oviposition (Stages X-XIII EG&K). In certain embodiments, the optogenetic system is activated to induce the lethality-promoting protein at Stages X. In certain embodiments, the optogenetic system is activated at Stages XI. In certain embodiments, the optogenetic system is activated at Stages XII. In certain embodiments, the optogenetic system is activated at Stages XIII. In certain embodiments, the optogenetic system is activated at any given time during embryonic development starting at fertilization until hatching. In certain embodiments, the lethality is induced during the 21 days period from fertilization to hatching. In certain embodiments, the lethality is induced 1 day after fertilization. In certain embodiments, the lethality is induced 2 days after fertilization. In certain embodiments, the lethality is induced 3 days after fertilization. In certain embodiments, the lethality is induced 4 days after fertilization. In certain embodiments, the lethality is induced 5 days after fertilization. In certain embodiments, the lethality is induced 6 days after fertilization. In certain embodiments, the lethality is induced 7 days after fertilization. In certain embodiments, the lethality is induced 8 days after fertilization. In certain embodiments, the lethality is induced 9 days after fertilization. In certain embodiments, the lethality is induced 10 days after fertilization. In certain embodiments, the lethality is induced 11 days after fertilization. In certain embodiments, the lethality is induced 12 days after fertilization. In certain embodiments, the lethality is induced 13 days after fertilization. In certain embodiments, the lethality is induced 14 days after fertilization. In certain embodiments, the lethality is induced 15 days after fertilization. In certain embodiments, the lethality is induced 16 days after fertilization. In certain embodiments, the lethality is induced 17 days after fertilization. In certain embodiments, the lethality is induced 18 days after fertilization. In certain embodiments, the lethality is induced 19 days after fertilization. In certain embodiments, the lethality is induced 20 days after fertilization. In certain embodiments, the lethality is induced 21 day after fertilization. In certain embodiments, the optogenetic system is activated during chicken embryo development after up to 30 hours of incubation. In certain embodiments, the optogenetic system is activated during chicken embryo development after up to 31 hours of incubation. In certain embodiments, the optogenetic system is activated during chicken embryo development after up to 32 hours of incubation. In certain embodiments, the optogenetic system is activated during chicken embryo development after up to 33 hours of incubation. In certain embodiments, the optogenetic system is activated during chicken embryo development after up to 34 hours of incubation. In certain embodiments, the optogenetic system is activated during chicken embryo development after up to 35 hours of incubation. In certain embodiments, the optogenetic system is activated during chicken embryo development after up to 36 hours of incubation. In certain embodiments, the optogenetic system is activated within the bird. In certain embodiments, the optogenetic system is activated prior to the formation of an eggshell surrounding the embryo. In certain embodiments, the optogenetic system is activated within the bird prior to the formation of an eggshell surrounding the embryo. In certain embodiments, the optogenetic system is activated by directly contacting the embryo with the inducer. In certain embodiments, the inducer is inserted into the egg. In certain embodiments, the eggshell is first at least partly opened, and the inducer is then administered directly to the embryo.

To validate induction of cellular death in chicken cells, the DTA and the two forms of caspase 3 were cloned in an expression vectors with the PGK (Phosphoglycerate Kinase) or CAGG promoters followed by TRES GFP which served to confirm the transfection efficiency. The PGK promoter is weaker and slower than the pCAGG in chicken embryonic cells.

In one embodiment, the vectors created were: PGK-DTA-TRES-GFP, pCAGG-DTA-IRES-GFP, PGK-CASP3-IRES-GFP, pCAGG-CASP3-IRES-GFP, PGK-mCASP3-IRES-GFP, pCAGG-mCASP3-IRES-GFP. The expression vectors without the lethality-inducing genes were used as controls (PGK-TRES-GFP and pCAGG-TRES-GFP).

The expression vectors were transfected to PGCs by electroporation and the cells were incubated for 24, 48 and 72 h before being analyzed using flow cytometry which detected positively expressing cells by the green fluorescence protein (GFP) and dead cells that were identified using Propidium Iodide (PI) staining. The nucleotide sequence encoding the GFP is set forth in SEQ ID NO:115.

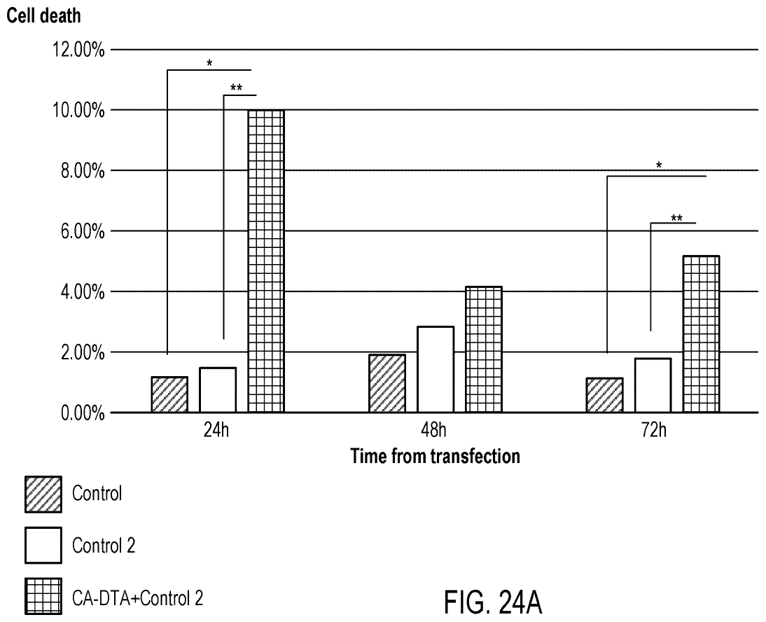

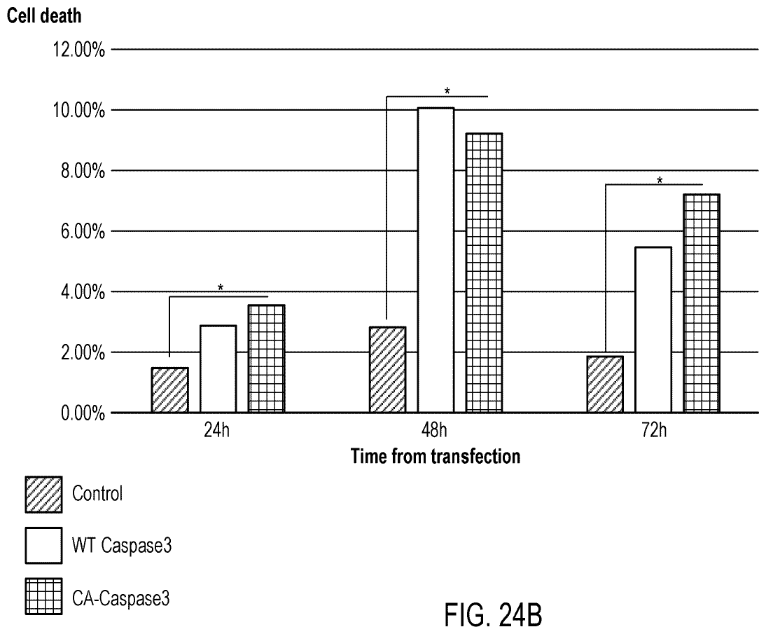

The results presented in FIGS. 24A-24B demonstrate the proportion of dead cells out of the total GFP positive cells.

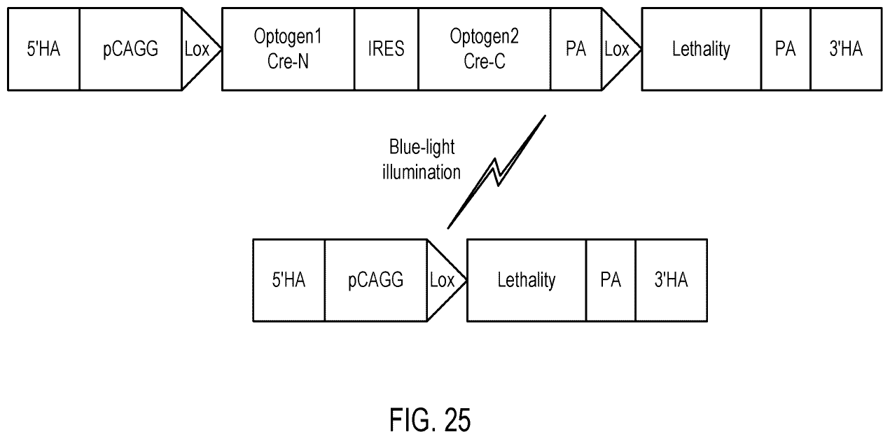

In one embodiment, a targeting vector consolidating the above described three elements into a single active unit which can be integrated to the Z chromosome was constructed. As shown in FIG. 25, the unit is flanked by the 5′ and 3′ homology arms. The promoter (pCAGG) drives the expression of the two units of the optogenetic system which are separated by an TRES sequence. The optogenetic system is followed by a polyadenylation sequence (PA). The optogenes cassette is flanked by two LoxP sites. The optogenetic system can be based on the CIBN-Cry2, the MAGNET or any other system as described above. Following the second LoxP site is the lethality-inducing coding sequence which is followed by a second polyadenylation sequence. As this lethality-inducing coding sequence has no promoter, it is inactive until a blue-light illumination is applied which results in the dimerization of the optogenes and activation of the Cre enzyme which removes the optogenes cassette flanked between the LoxP sites. This excision results in placing the lethality coding sequence directly downstream to the pCAGG promoter which drives its expression. This strategy benefits from a shorter design since it contains a single promoter and does not require “STOP” sequence.

In one embodiment, the nucleotide sequence for the pCAGG promotor is set forth in SEQ ID NO:100. The nucleotide sequence for the pGK promotor is set forth in SEQ ID NO:109. The nucleotide sequence for the pCMV promotor is set forth in SEQ ID NO:110. The nucleotide sequence for the phSyn promotor is set forth in SEQ ID NO:111. The nucleotide sequence for the pEFI-a promotor is set forth in SEQ ID NO:112.

In one embodiment, the nucleotide sequence for Optogen 1—NLS-Cry2-A535-L348F-CreN (AA 19-104) is set forth in SEQ ID NO:101. The nucleotide sequence for Optogen 2-CIBN(aal-170)-NLS-Cre-C(aa106-343) is set forth in SEQ ID NO:102. The nucleotide sequence for Cre recombinase is set forth in SEQ ID NO:113. The nucleotide sequence for CreN (AA 19-104) is set forth in SEQ ID NO:117. The nucleotide sequence for CreC (AA 106-343) is set forth in SEQ ID NO:118.

In one embodiment, the nucleotide sequence for the IRES sequence is set forth in SEQ ID NO:103. The nucleotide sequence for the polyadenylation site sequence (Rabbit beta-globin) is set forth in SEQ ID NO:104. The nucleotide sequence for the 5′ homology arm (LHA) is set forth in SEQ ID NO:105. The nucleotide sequence for the 3′ homology arm (RHA) is set forth in SEQ ID NO:106.

In one embodiment, regarding the MAGNET system, the following sequences are alternative optogens that can be used instead of the Cry2-CIBN system. For example, the nucleotide sequence for the CreN(aal8-59)_N-Mag_NLS is set forth in SEQ ID NO: 107. The nucleotide sequence for the NLS_P-Mag_Cre-C(aa60-237) is set forth in SEQ ID NO: 108.

In one embodiment, transplantation of modified PGCs to embryos and producing chimera chickens that will be screened for germ-line transmission and potential founder carriers were carried out as follows. Following the generation of the targeting vector, PGC lines, which originated from both female and male embryos, underwent co-electroporation with px330-all-in-one CRISPR-Cas9-GFP encoding plasmid and the targeting vector. The CRISPR plasmid creates a DNA double-strand break at the designed site in chromosome Z, thus facilitating homologous recombination of the targeting vector. The CRISPR plasmid encodes Cas9 fused in-frame with GFP, thus allowing identification of successful transfection. 24-72 hrs following transfection, positive single cells were FACS sorted to single cells in 96-well plate and were grown to form pure PGCs colonies which were screened by PCR and southern blot to identify colonies which underwent proper homologous recombination.

Chimera chickens were generated as follows. The chimera chickens are a mean to transform genetically modified PGCs into functional gametes. To generating chimeras, stage 14-16 H&H embryos were injected to the bloodstream (e.g. to the heart) with >3000 PGCs (normally up to 8000-10000). These cells colonized the embryonic gonads alongside the endogenous PGCs, thus they are referred as chimeras. The efficiency of germline transmission, i.e. the ability to transform genetically modified PGCs to functional PGCs, rely on several factors including the ratio between the endogenous and injected PGCs in the gonad. In order to reduce the amount of endogenous PGCs, freshly-laid fertile eggs are γ irradiated with 600-800 rad. In certain embodiments, the irradiation comprises 600-800 rad of irradiation. In certain embodiments, the irradiation comprises 400-1000 rad of irradiation. In certain embodiments, the irradiation comprises 200-1200 rad of irradiation.

It is thought that PGCs are more susceptible to γ irradiation than somatic cells or that following irradiation the PGCs are less competent to regenerate as somatic cells do. Thus, after irradiation the total amount of endogenous PGCs is reduced. Following irradiation, the eggs were normally incubated until the embryos reach stage 14-16 H&H which is adequate for PGCs injection. As an indication for the efficiency of the irradiation, the incubation time required for the embryos to reach stages 14-16 H&H increases in about 10 h and as much as 70% of the irradiated embryos fail do normally develop and thus are not being used for PGCs injection. Different chicken strains, eggshells type, thick and color may require different irradiation conditions, thus the amount of energy warrants for calibration. Following Injection, the eggs are sealed and incubated until the chimera chicks hatch. The chicks are then reared to sexual maturity and in males, sperm is collected for analysis of germline transmission using semi-quantitative PCR or Real-Time PCR.

### Example 3

The present example presents targeting vectors containing an addition element, an element referred herein as the “safe-lock” element. This element basically locks the optogenic-lethality system, so by default, the system is inactive. Only by crossing with “safe-unlocking” strain would the optogenic-lethality system become active. This element benefits the entire system by better protecting the breeds, since in order to be activated there is a need to use additional “unlocking” strain. This also ensures that throughout the production process, cells which have undergone HR need not be protected from light, since the optogenic system is essentially inactive.

The present example discloses eight “All-in-one” targeting vectors, and demonstrates both in-vitro and in-vivo that they work as designed. The data also show that the MAGNET optogenic system is working in chicken embryos and is able to activate the lethality-inducing mechanism. The MAGNET system has been described above. Moreover, the data show that Noggin, as an embryonic lethality-inducing gene, stops embryonic development from oviposition (from the freshly-laid eggs).

The eight targeting vectors disclosed herein cover 8 combinatorial options of 2 optogenic systems (MAGNET and CIBN-Cry2) and 4 lethality-inducing genes (DTA, Noggin, caCASP3, and mCherry, which is used for control and verification process). To clarify, mCherry as such does not induce lethality and was used to confirm the action of the other elements.

As discussed above, the aim of the experiments described herein is to introduce a lethality-inducing cassette into the Z chromosome of either male or female-derived PGCs. The final goal is to obtain hens harboring a genomic insertion on the Z chromosome. This chromosome will segregate only to the next generation male embryos that upon blue-light induction will activate a lethality-inducing cassette, thus the male embryos will die at early stages of embryogenesis. Therefore, due to sex chromosomal segregation, the female laying hen does not acquire modified genetic material, since it gets a WT Z chromosome from the male rooster side and WT W chromosome from the mother hen side (see FIG. 1). In this example, the targeting vectors comprise 4 elements as described below (see examples in FIG. 26).

As shown in FIG. 26, the first element is the “Homology arms”. In one embodiment, two ~1.5 kb homology arms are located on both 5′ and 3′ ends of the vector. This is designed to direct the homologous recombination (HR) downstream to the HINT1Z locus located on the Z chromosome. This site for HR was chosen since the HINT1Z gene is transcribed in PGCs as well as in whole blastoderms (these are freshly laid chick embryos). All other openly transcribed regions on the Z chromosome are also potentially good candidates for this purpose.

The second element is a “safe-lock” mechanism which ensures that the optogenic-lethality mechanism is inactive until the STOP cassette is removed. The STOP cassette is flanked by two FRT sites and it is located between the pCAGG promoter and the optogenes, thereby preventing the expression of the optogenes. Upon crossing with a Flp expressing strain as described below, the STOP element is removed, allowing the optogenes to be transcribed and become active in a light-dependent manner (FIG. 26A, active “unlocked” state). In this example, the “safe-lock” element comprises the coding sequences for GFP followed by a polyadenylation site. Any other sequences which will prevent the transcription of downstream elements can also be used as a “safe lock” element.

The third element is the “Optogenetic inducible element”. As discussed above, optogenetic systems are based on proteins which under certain light wavelengths will be excited and change conformation to dimerize with a second specific protein. Examples of optogenetic systems have been discussed above.

In this example, two alternative optogenic systems were tested and used. The first, the MAGNET system, in which two optogenetic proteins—positively charged P-Mag and negatively charged N-Mag dimerize upon blue light illumination. Sequences encoding the site-specific recombinase enzyme Mag are listed in SEQ ID NO:114 and SEQ ID NO:65. Each of these proteins is fused to an inactive part of the Cre recombinase enzyme and upon dimerization of the two optogenes, an active form of Cre is created (see e.g. Nature Chemical Biology volume12, 1059-1064 2016). In this case, sequences for a self-cleaving peptide P2A (“Link” in FIG. 26) are located between the two optogenes.

The second optogenic system that was used is an improved system of the CIBN(CreC)-Cry2(CreN). In this case a modification includes an insertion of L348F mutation in a truncated Cry2 gene which contains only the first 535 amino acids—Cry2-A535-L348F (see Nature Chemical Biology 25 volume12, pages425-430 (2016). In one embodiment, the linker between the two optogenes is an TRES sequence (“Link” in FIG. 26).

The fourth element is the “lethality-inducing cassette”. In one embodiment, the lethality-inducing cassette comprises a gene which promotes early embryonic death by means of cell death or severe intervention in molecular signaling pathway required for early stages of embryogenesis, such as the BMP4. As discussed above, examples of the lethality-inducing genes include, but not limited to, toxins such as the Diphtheria toxin A (DTA), Caspase genes such as Caspase3 or a mutated constitutively-active mutated form of Caspase 3, or the inhibitor protein of BMP4-Noggin.

The table below lists the features of these eight targeting vectors. In one embodiment, these vectors were cloned into pJet1.2 shuttle vector plasmid.

| Targeting | | Optogenes | Lethality | SEQ |
|---|---|---|---|---|
| Vector | Optogenic system | linker | gene | ID NO |
| TV1 | Negative MAGNET-Positive | P2A | Noggin | 120 |
| | MAGNET | | | |
| TV2 | Negative MAGNET-Positive | P2A | DTA | 121 |
| | MAGNET | | | |
| TV3 | Negative MAGNET-Positive | P2A | caCASP3 | 122 |
| | MAGNET | | | |
| TV4 | Negative MAGNET-Positive | P2A | mCherry | 123 |
| | MAGNET | | | |
| TV5 | [CIBN-CreC]-[Cry2-Δ535- | IRES | Noggin | 124 |
| | L348F-CreN] | | | |
| TV6 | [CIBN-CreC]-[Cry2-Δ535- | IRES | DTA | 125 |
| | L348F-CreN] | | | |
| TV7 | [CIBN-CreC]-[Cry2-Δ535- | IRES | caCASP3 | 126 |
| | L348F-CreN] | | | |
| TV8 | [CIBN-CreC]-[Cry2-Δ535- | IRES | mCherry | 127 |
| | L348F-CreN] | | | |

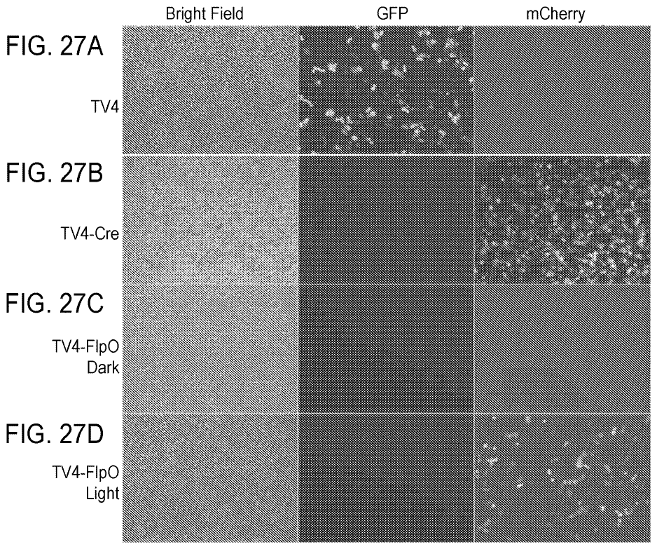

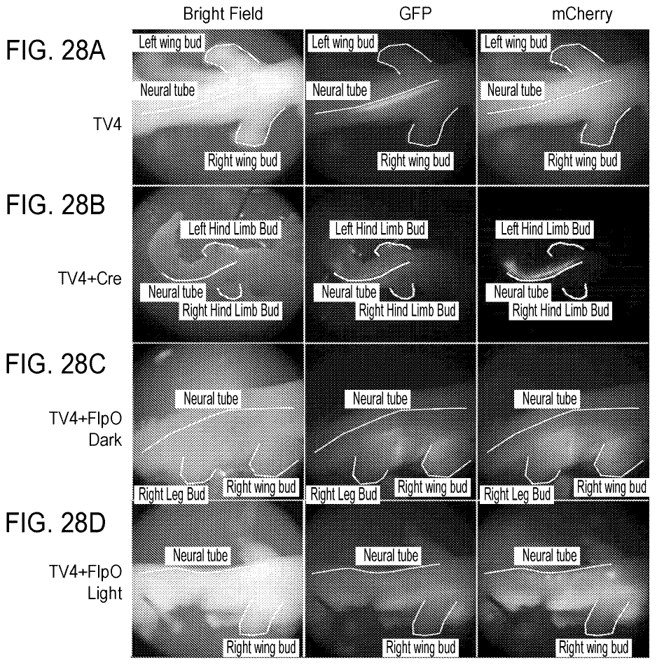

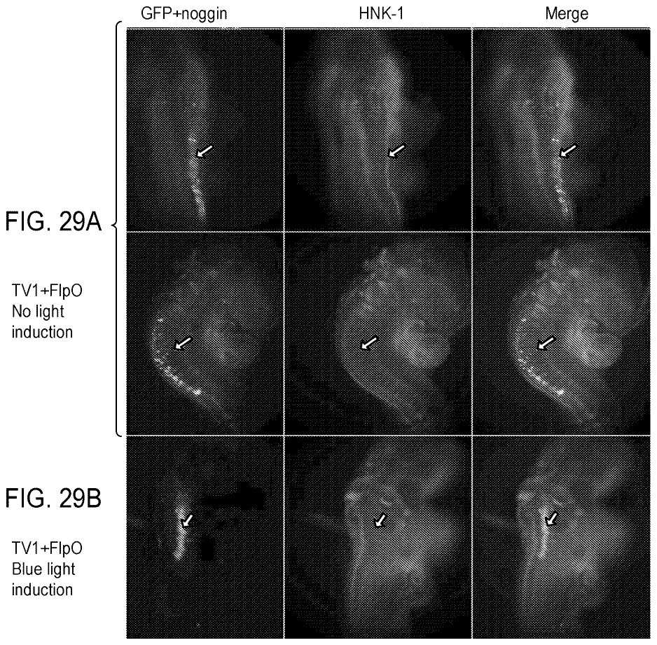

The activity of the elements in the targeting vectors (TVs) was assessed in-vitro in HEK293 cells (FIG. 27) and in-ovo in chicken embryos (FIGS. 28-29). For in-vitro validation, HEK293 cells were transfected with TV4 alone (FIG. 27A), with pCAGG-Cre (SEQ ID NO:128) (FIG. 27B), or with pCAGG-FlpO (SEQ ID NO:129) plasmids. The latter was done under two treatments, one was kept in dark (FIG. 27C) and the other was exposed for 15 seconds to blue light, 24 hours following transfection. Following illumination, the cells were further incubated for 24 hours (FIG. 27D).

When expressed alone, TV4, like all other TVs, expressed GFP, thus indicating the activity of the “safe-lock” state (FIG. 27A). When co-expressed with pCAGG-Cre plasmid, the “safe-lock” and the optogenes cassettes were removed altogether (FIG. 26B; FIG. 27B), which was indicated by the loss of expression of the GFP and the onset of mCherry expression (as a surrogate for the lethality inducing gene). This result confirms that upon excision of the “safe-lock” and optogenes elements, the lethality-inducing element becomes active. When TV4 was co-expressed with pCAGG-FlpO plasmid, only the “safe lock” element was removed, as indicated by the loss of expression of the GFP (FIG. 26A, FIGS. 27C-D), and the optogenic system became active in a light-dependent manner. There was no activation of the optogenic system and no expression of mCherry when the cells were kept in the dark, indicating the lethality cassette was not expressed (FIG. 27C). However, under the same conditions, when the cells are illuminated, the optogenic system became active and was excised (see FIG. 26A) to allow the lethality element to be expressed. This is indicated by the expression of mCherry as shown in FIG. 27D.

To demonstrate the activity of the TV elements in-vivo, chicken embryos were injected with plasmids to the neural tube and electroporated as described above. In FIG. 28, the white lines denote the dorsal mid-line of the neural tube and limb buds for orientation purposes. Four treatment groups were tested: 1. expression of TV4 alone (FIG. 28A), 2. co-electroporation of TV4 and pCAGG-Cre plasmids, as positive control (FIG. 28B), 3. Co-electroporation of TV4 and pCAGG-FlpO plasmids, which were kept in dark (FIG. 28C), and 4. Exposure to blue light for 15 sec, following co-electroporation of TV4 and pCAGG-FlpO plasmids, and further incubation for 12 hours (FIG. 28D).

When electroporated alone, TV4 expressed GFP to indicate the default inactive “safe-lock” state and no mCherry was expressed (FIG. 28A). When co-expressed with pCAGG-Cre plasmid, the “safe-lock” and the optogenes cassettes were removed (FIG. 26B; FIG. 28B), thus there was no GFP expression but mCherry was expressed. This serves as a positive control for activation of the lethality-inducing elements. By co-expressing TV4 with pCAGG-FlpO plasmid, only the FRT-flanked “safe lock” element was removed, thus there was no GFP expression (FIGS. 28C-28D) but the optogenic system was activated in light-dependent manner. There was no GFP expression when the cells were kept in the dark, indicating the optogenic system was in its active state, yet mCherry was not expressed, i.e. the lethality cassette was inactive (FIG. 28C). However, when the embryos were illuminated 12 hours following electroporation, the optogenic system became active and was excised to allow the lethality element to be expressed. This is indicated by the expression of mCherry as shown in FIG. 28D.

To demonstrate the activity of the lethality-inducing gene Noggin in a light-dependent manner, TV1, which contains the coding sequence of Noggin as a lethality-inducing element, was co-electropotrated with pCAGG-FlpO plasmid to the rostral neural tube (axial level of the mid-brain and hind-brain) of chick embryos that had been incubated for 36 hours. At this stage, neural-crest cells delaminate from the dorsal neural tube in a BMP4-dependent manner. Thus, inhibiting the BMP4 signaling pathway by ectopic expression of Noggin in the dorsal neural tube is predicted to inhibit neural crest cells delamination. In order to visualize neural crest cells, anti-HNK-1 antibodies were used for staining of the neural crest marker HNK-1.

FIG. 29 shows embryos that were electroporated in the neural tube with TV1 (SEQ ID NO:120), pCAGG-FlpO (SEQ ID NO:129) and pCAGG-IRES-GFP (SEQ ID NO:59) plasmids. The latter was added as a positive control to allow monitoring of transfected cells. Twenty four hours after transfection, in embryos that were kept in the dark throughout the experiment (FIG. 29A, upper row, dorsal view, lower row, right-lateral view), neural crest cells delaminated and migrated normally from the dorsal neural tube. The HNK-1 staining revealed bi-lateral symmetry expression pattern in the dorsal tube on both right and left neural folds (the dorsal-most aspect of the neural tube that gives rise to migrating neural crest cells), as well as on migrating neural crest cells (FIG. 29A, arrowhead). However, in embryos that were exposed to light 12 hours after electroporation, neural crest cells failed to delaminate and migrate from the tube, and HNK-1 staining revealed marked reduction in expression on the right neural fold—at the electroporated side (FIG. 29B, arrow). Thus, these results indicate that upon removal of the “safe lock” element from TV1, Noggin expression and activity is regulated in a light-dependent manner by the MAGNET optogenic system.

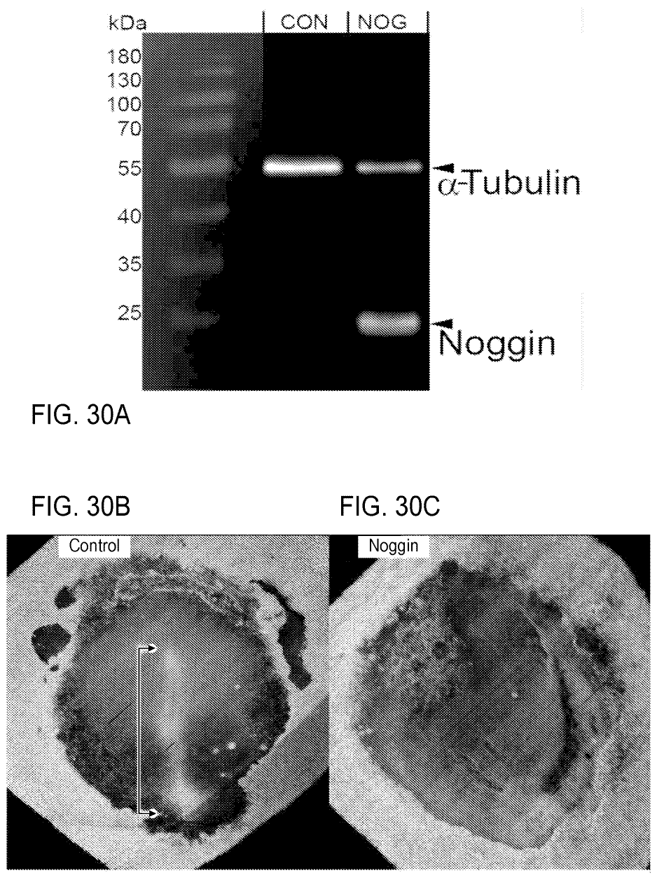

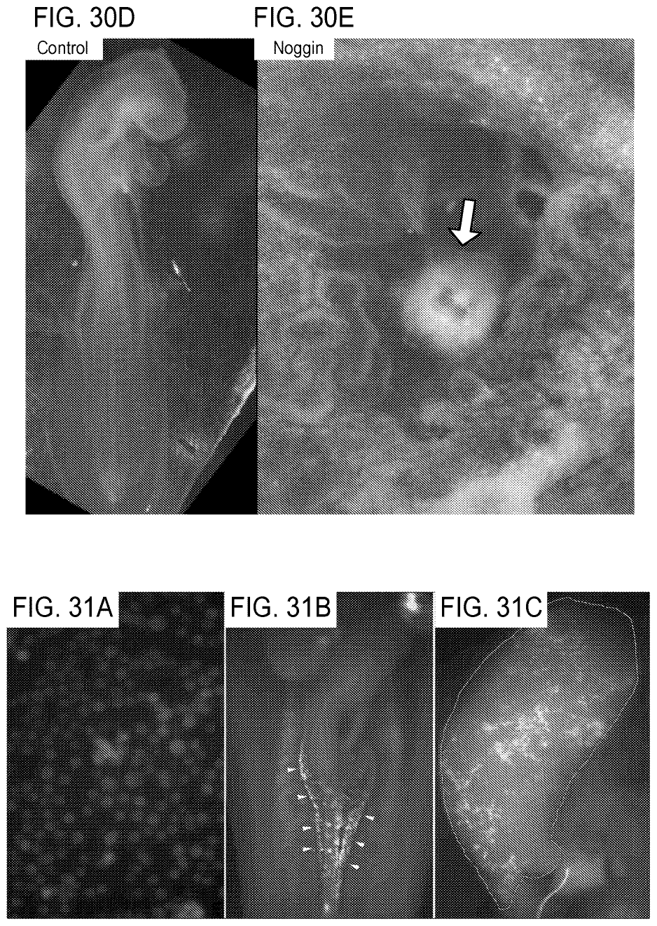

To demonstrate that Noggin is able to stop embryonic development as soon as the egg is laid, at the blastoderm embryonic stage, blastoderms were treated with exogenous source of Noggin, in-ovo (FIG. 30). To this end, pCAGG-Noggin-IRES-GFP (SEQ ID NO:130), or as negative control, pCAGG-IRES-GFP (SEQ ID NO:59) plasmids were transfected to HEK293 cells. To validate the expression of Noggin in transfected cells, total protein extracted from transfected cells was analyzed by Western blot with anti-Noggin antibody (Abcam, ab16054, predicated size ~24 kDa) and anti-α-Tubilin-HRP antibody (Abcam, ab40742, predicted size ~55 kDa) as loading control. FIG. 30A shows that while there was no Noggin expression in the negative control transfected cells (CON), cells transfected with pCAGG-Noggin-IRES-GFP (NOG) produced noggin. Prestained protein ladder was presented in the left lane (Thermo Scientific, PageRuler #26617). Conditioned media from control and Noggin expressing cells were injected into freshly-laid fertile eggs that were subsequently incubated for 24 or 54 hours, as shown in FIG. 30B-C and FIG. 30D-E, respectively. FIG. 30B-C show a High-Resolution Episcopic Microscopy (HREM) 3D reconstructed models of treated embryos. While control-treated embryos (FIG. 30B) continued to develop normally and underwent normal gastrulation, as indicated by the formation of the Primitive Streak (demarcated by arrows), Noggin-treated embryos failed to gastrulate and had no apparent Primitive Streak, indicating that the process of embryogenesis essentially stopped (FIG. 30C). When the embryos were left to further develop for 54 hours of incubation (FIG. 30D-E), control-treated embryos developed normally, underwent normal neurulation process and formed a beating heart (FIG. 30D), whereas Noggin-treated embryos formed a mass of cells with no overt characteristics (FIG. 30E, arrow).

Collectively, the results described above show that the molecular mode-of-action strategy presented in FIG. 26 works as predicted, including the “Safe-lock” elements and MAGNET optogenic system that activates the lethality-inducing element in a light-dependent manner. Additionally, inhibiting the BMP signaling pathway by treating blastoderms in freshly-laid eggs with Noggin stopped the progression of embryonic development.

### Creating Pure Lines of Pgcs Harboring an Integrated Targeting Vector on the Z Chromosome

In one embodiment, to generate PGCs with targeted integration to the Z chromosome, the ribonucleoprotein (RNP) system for delivering CRISPR/Cas9 was used. The use of RNP system benefits, among other things, from high efficiency, rapid DNA cleavage and rapid clearing of the RNP complexes from transfected cells. RNP complexes comprise of recombinant Cas9 or High-fidelity Cas9 nucleases and mixture of crRNA:tracrRNA complexes. The tracrRNA are commercially available (IDT), and the crRNA can be custom-made with specific 20 nucleotide sequence corresponding to the desired cleavage site on the targeted genomic DNA. In one embodiment, SEQ ID 66 and SEQ ID 68 disclosed herein were used to synthesize two crRNA oligos (see FIG. 8).

PGCs culture medium and lines derivation has been described above. To electroporate PGCs, 5×10⁵ cells were washed in AkoDMEM, then transferred to buffer “R” (Neon buffer, Invitrogen) containing 0.7 μg of the targeting vector plasmid (in this example TV1), 1 μM Alt-R® cas9 electroporation enhancer (IDT), and RNP complexes with final concentration of 1.5 μM recombinant Cas9 or High fidelity Cas9 nucleases (Alt-R® S.p. Cas9 Nuclease or S.p. HiFi Cas9 Nuclease V3, respectively; IDT) and 1.8 μM crRNA:tracrRNA (sgRNA complexes, Custom Alt-R® CRISPR-Cas9 crRNA and Alt-R® tracrRNA; IDT).

The crRNA:tracrRNA complexes were prepared by heating the RNA oligos to 95 °C for 1 minute, and cooling down to room temperature. RNP complexes (Cas9 protein+crRNA:tracrRNA) mixture was prepared by adding recombinant Cas9 or high-fidelity Cas9 nucleases proteins to the crRNA:tracrRNA complexes, and incubating at room temperature for 10-20 min. It is generally believed that high-fidelity Cas9 is supposed to cause less off-targets.

For electroporation, 10 μl of buffer “R”, containing the RNP complexes, electroporation enhancer reagent and the targeting vector plasmid, were added to PGCs pellet and the mixture was immediately electroporated with 3 pulses of 1000V, 13 ms duration each, using Neon electroporator (Invitrogen). Subsequently, the cells were immediately seeded in 48-wells plate in PGCs medium containing 1 μM SCR7-pyrazine (SML1546 sigma). Medium was changed after 1-4 hours, and transfected cells were allowed to recover for 7-10 days. SCR7-pyrazine was added for 48 h after electroporation. Transfected cells were individually isolated by FACS sorting. For FACS sorting, gentle cells pipetting was done and cells were sorted in PGCs culture medium. Single GFP positive cells were sorted with FACS Sony sorter (Sony) to a U-shape 96 wells plate. Sorted cells were grown for 2-3 weeks to form pure colonies. From these colonies, total genomic DNA was extracted for analysis, cryopreserved and positive colonies were injected to surrogate host embryos. PCR and Southern blot analysis used to verify correct integration of the targeting vector have been described above (see e.g. FIG. 15-16). Methods of creating surrogate chimera chicks have also been described above.

FIG. 31A shows pure female PGCs line that underwent HR with TV1, and expressed GFP. Cells from this colony were injected to surrogate host embryos that were incubated for 5 day following transplantation. FIG. 31B shows a ventral view of an embryo 5 days following PGCs injection. At this stage, the PGCs colonized the genital ridge which is the anlage of the gonads (FIG. 31B, arrowheads). Other injected embryos were incubated until hatch, and female chicks were sacrificed at day 10 post hatch to analyze the ovary. FIG. 31C shows an ovary (delineated by a line) containing numerous GFP positive PGCs. These results indicate that the PGCs that underwent HR on the Z chromosome with TV1 successfully colonize the gonads.

### Creating “Unlocking” Strains Expressing Cre or Flpo.

Transgenic chicken strains expressing the targeting vectors TV1-8 will have the “safe-lock” element in their genome, hence the optogenic-lethality system will be inactive. In order to remove the “safe-lock” element which is flanked by FRT sites (see FIG. 26A), these strains need to be crossed with FlpO-expreesing chicken strain. To generate this strain, a targeting vector containing the FlpO enzyme followed by IRES-GFP was created (TV-FlpO-IRES-GFP, SEQ ID NO:131). As a positive control and as a mean to activate the lethality-inducing cassette in a light-independent manner, a Cre-expressing breed can be used. In this case, the “safe-lock” and optogenic elements, which are flanked by LoxP sites, are removed to activate the lethality inducing element (see FIG. 26B). To generate this strain, a targeting vector containing the Cre enzyme followed by P2A-GFP was created (TV-Cre-P2A-GFP, SEQ ID NO:132). These two targeting vectors share the same 5′ and 3′ homology arms as well as the reporter gene GFP, and are designed to integrate to the Z chromosome as described above for TV1-8.

The above targeting vectors (TV-FlpO-IRES-GFP and TV-Cre-P2A-GFP) have been used to generate PGCs that were injected into surrogate chimera embryos. Methods of transfection of PGCs, FACS analysis, verification of HR integration, embryos injections, etc. have been described above.

In another embodiment, there is an option of using recombinant CRE or FlpO proteins to remove the “safe-lock” element. In one embodiment, sequences encoding the recombinant proteins contain sequences for TAT peptide, which is a cell-penetration peptide, followed by Nuclear Localization Sequence (NLS), which in turn is followed by sequences encoding either the Cre or FlpO enzymes can be applied to the cells or embryos. These recombinant proteins are effective in cultured cells, and they are commercially available. It is believed that these recombinant proteins can be injected into embryos or to adults and potentially activate the system without the need of generating Cre/Flp transgenic chickens and crossing. The accessibility of the chick embryo through an opening on the egg shell allows direct injection to the blood stream or in the vicinity of the embryo. Thus, in one embodiment, the above recombinant proteins can be directly injected into the embryo to remove the “safe-lock” element.

Although certain embodiments have been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

While certain features of aspects or embodiments have been illustrated and described herein, many modifications, substitutions, changes, and equivalents will now occur to those of ordinary skill in the art. It is, therefore, to be understood that the appended claims are intended to cover all such modifications and changes as fall within the true spirit of the aspects or embodiments provided herein.

1. A DNA editing agent, said DNA editing agent comprising a polynucleotide cassette having a formula 5′-LHA (left homology arm)-OIE (optogenetic-inducible element)-LIE (lethality-inducing element)-RHA (right homology arm)-3′, wherein
(i) the LHA comprises a first nucleotide sequence that is substantially homologous to a first corresponding nucleotide sequence on chromosome Z of a bird;
(ii) the OIE comprises a first promoter functionally linked to a second nucleotide sequence encoding an inducer-activated site-specific recombinase enzyme, wherein the inducer-activated site-specific recombinase enzyme comprises non-functional peptide fragments of an inducer-activated site-specific recombinase enzyme that combine to form an active inducer-activated site-specific recombinase enzyme in the presence of the inducer;
(iii) the LIE comprises a third nucleotide sequence encoding a lethality-promoting protein, which is operatively linked to the activity of the inducer-activated site-specific recombinase enzyme; and
(iv) the RHA comprises a fourth nucleotide sequence that is substantially homologous to a second corresponding nucleotide sequence on chromosome Z of a bird, wherein the first promoter is not functionally linked to the third nucleotide sequence.

2. The DNA editing agent of claim 1, wherein the first corresponding nucleotide sequence or the second corresponding nucleotide sequence is located in an openly transcribed region on chromosome Z.

3. The DNA editing agent of claim 1, wherein the inducer-activated site-specific recombinase enzyme comprises Cre recombinase or Mag recombinase.

4. The DNA editing agent of claim 1, wherein expression of the inducer-activated site-specific recombinase enzyme is induced by an inducer, wherein the inducer is electromagnetic energy.

5. The DNA editing agent of claim 1, wherein the inducer-activated site-specific recombinase enzyme is a CRISPR associated protein (CAS) endonuclease.

6. The DNA editing agent of claim 1, wherein the polynucleotide cassette further comprises a fifth nucleotide sequence that encodes for a guide RNA that targets an essential gene of the bird, the fifth nucleotide sequence being operatively linked to the activity of the inducer-activated site-specific recombinase enzyme.

7. The DNA editing agent of claim 1, further comprising a safe-lock element inserted downstream to the promoter in the OIE but upstream of the sequence encoding the inducer-activated site-specific recombinase, said safe-lock element comprises nucleotide sequences (STOP element) that prevent transcription of the inducer-activated site-specific recombinase encoded by the OIE.

8. A DNA editing agent, said DNA editing agent comprising a polynucleotide cassette having a formula 5′-LHA (left homology arm)-LIE (lethality-inducing element)-OIE (optogenetic-inducible element)-RHA (right homology arm)-3′, wherein
(i) the LHA comprises a first nucleotide sequence that is substantially homologous to a first corresponding nucleotide sequence on chromosome Z of a bird;
(ii) the OIE comprises a first promoter functionally linked to a second nucleotide sequence encoding an inducer-activated site-specific recombinase enzyme, wherein the inducer-activated site-specific recombinase enzyme comprises non-functional peptide fragments of an inducer-activated site-specific recombinase enzyme that combine to form an active inducer-activated site-specific recombinase enzyme in the presence of the inducer;
(iii) the LIE comprises a third nucleotide sequence encoding a lethality-promoting protein, which is operatively linked to the activity of the inducer-activated site-specific recombinase enzyme; and
(iv) the RHA comprises a fourth nucleotide sequence that is substantially homologous to a second corresponding nucleotide sequence on chromosome Z of a bird, wherein the first promoter is not functionally linked to the third nucleotide sequence.

9. The DNA editing agent of claim 8, wherein the first corresponding nucleotide sequence or the second corresponding nucleotide sequence is located in an openly transcribed region on chromosome Z.

10. The DNA editing agent of claim 8, wherein the inducer-activated site-specific recombinase enzyme comprises Cre recombinase or Mag recombinase.

11. The DNA editing agent of claim 8, wherein expression of the inducer-activated site-specific recombinase enzyme is induced by an inducer, wherein the inducer is electromagnetic energy.

12. The DNA editing agent of claim 8, wherein the inducer-activated site-specific recombinase enzyme is a CRISPR associated protein (CAS) endonuclease.

13. The DNA editing agent of claim 8, wherein the polynucleotide cassette further comprises a fifth nucleotide sequence that encodes for a guide RNA that targets an essential gene of the bird, the fifth nucleotide sequence being operatively linked to the activity of the inducer-activated site-specific recombinase enzyme.

14. The DNA editing agent of claim 8, further comprising a safe-lock element inserted downstream to the promoter in the OIE but upstream of the sequence encoding the inducer-activated site-specific recombinase, said safe-lock element comprises nucleotide sequences (STOP element) that prevent transcription of the inducer-activated site-specific recombinase encoded by the OIE.

15. A DNA comprising 5′ to 3′:
(i) a 5′ homology arm that targets a 5′ region flanking a target gene locus in Z chromosome of an avian;
(ii) a first polynucleotide comprising 5′ to 3′: a first promoter operably linked to (a) a nucleic acid sequence encoding a fusion protein comprising cryptochrome 2 (CRY2) and a N-terminal region of a recombinase, and (b) a nucleic acid sequence encoding a fusion protein comprising a truncated calcium and integrin-binding protein (CIBN) and a C-terminal region of a recombinase;
(iii) a second polynucleotide comprising 5′ to 3′: a second promoter operably linked to (a) a loxP-Stop-loxP site, and (b) a nucleic acid sequence encoding a lethality protein,
and
(iv) a 3′ homology arm that targets a 3′ region flanking the target gene locus;
wherein said DNA is capable of integrating into the target gene locus,
wherein, upon integration into said region, when said DNA is exposed to blue light, said DNA expresses a CRY2/CIBN heterodimer and an active recombinase,
wherein said active Cre recombinase is capable of excising said loxP-Stop-loxP site, thereby allowing expression of said lethality protein.

16. The DNA of claim 15, wherein the DNA is operatively linked to a recombinase recognition site.

17. The DNA of claim 15, wherein the recombinase is a Cre recombinase.

18. The DNA of claim 15, wherein the target gene locus is a histidine triad nucleotide binding proteins (HINT) gene.

19. The DNA of claim 15, wherein the lethality protein is a toxin.


SEQUENCE LISTING (132 sequences)

SEQ ID NO: 1 - DNA, 149 bp, synthetic construct
  misc_feature 1..149: note = HA1-CRISPER-HA2
  source 1..149: mol_type = other DNA
aacacagcttatatacatttttacctacaaaatcgtgctgtcatgtcccactctgattggttcataccagataacgtgccttatttggccgtttccacattcttttctcatccttcttctcctgttttctctgcatcaaggtcagcacg

SEQ ID NO: 2 - DNA, 30 bp, synthetic construct
  misc_feature 1..30: note = CRISPER WT Sequence
  source 1..30: mol_type = other DNA
ataccagataacgtgccttatttggccgtt

SEQ ID NO: 3 - DNA, 32 bp, synthetic construct
  misc_feature 1..32: note = CRISPER Mutant Sequence
  source 1..32: mol_type = other DNA
ataccagataacgtaatccttatttggccgtt

SEQ ID NO: 4 - DNA, 20 bp, synthetic construct
  misc_feature 1..20: note = Primer 1
  source 1..20: mol_type = other DNA
ttttgaatgaagggcctgag

SEQ ID NO: 5 - DNA, 20 bp, synthetic construct
  misc_feature 1..20: note = Primer 2
  source 1..20: mol_type = other DNA
ggttggttcactgttgtctg

SEQ ID NO: 6 - DNA, 45 bp, synthetic construct
  misc_feature 1..45: note = Primer 3
  source 1..45: mol_type = other DNA
gtccctcttctcttatggagatcgccgtttccacattcttttctc

SEQ ID NO: 7 - DNA, 49 bp, synthetic construct
  misc_feature 1..49: note = Primer 4
  source 1..49: mol_type = other DNA
ggtggcacttttcggggaaatgtgtgaaccaatcagagtgggacatgac

SEQ ID NO: 8 - DNA, 49 bp, synthetic construct
  misc_feature 1..49: note = Primer 5
  source 1..49: mol_type = other DNA
gtcatgtcccactctgattggttcacacatttccccgaaaagtgccacc

SEQ ID NO: 9 - DNA, 45 bp, synthetic construct
  misc_feature 1..45: note = Primer 6
  source 1..45: mol_type = other DNA
gagaaaagaatgtggaaacggcgatctccataagagaagagggac

SEQ ID NO: 10 - DNA, 19 bp, synthetic construct
  misc_feature 1..19: note = Primer 7
  source 1..19: mol_type = other DNA
gaagtgtgctgctaacctg

SEQ ID NO: 11 - DNA, 20 bp, synthetic construct
  misc_feature 1..20: note = Primer 8
  source 1..20: mol_type = other DNA
gctatgaactaatgaccccg

SEQ ID NO: 12 - DNA, 21 bp, synthetic construct
  misc_feature 1..21: note = Primer 9
  source 1..21: mol_type = other DNA
ttttcctcctctcctgactac

SEQ ID NO: 13 - DNA, 22 bp, synthetic construct
  misc_feature 1..22: note = Primer 10
  source 1..22: mol_type = other DNA
ggcctggatgataagagtcttc

SEQ ID NO: 14 - DNA, 20 bp, synthetic construct
  misc_feature 1..20: note = Primer 11
  source 1..20: mol_type = other DNA
gctattcggctatgactggg

SEQ ID NO: 15 - DNA, 20 bp, synthetic construct
  misc_feature 1..20: note = Primer 12
  source 1..20: mol_type = other DNA
gaaggcgatagaaggcgatg

SEQ ID NO: 16 - DNA, 20 bp, synthetic construct
  misc_feature 1..20: note = Primer 13
  source 1..20: mol_type = other DNA
gtggaacacagcttttccag

SEQ ID NO: 17 - DNA, 20 bp, synthetic construct
  misc_feature 1..20: note = Primer 14
  source 1..20: mol_type = other DNA
gctcttcaacttgccatttg

SEQ ID NO: 18 - DNA, 19 bp, synthetic construct
  misc_feature 1..19: note = Primer 15
  source 1..19: mol_type = other DNA
tcaacagcacgtaagcaac

SEQ ID NO: 19 - DNA, 20 bp, synthetic construct
  misc_feature 1..20: note = Primer 16
  source 1..20: mol_type = other DNA
cctgactccatttttgagcc

SEQ ID NO: 20 - DNA, 22 bp, synthetic construct
  misc_feature 1..22: note = Primer 17
  source 1..22: mol_type = other DNA
cccaaatataacacgcttcact

SEQ ID NO: 21 - DNA, 23 bp, synthetic construct
  misc_feature 1..23: note = Primer 18
  source 1..23: mol_type = other DNA
gaaatgaattattttctggcgac

SEQ ID NO: 22 - DNA, 20 bp, synthetic construct
  misc_feature 1..20: note = Primer 19
  source 1..20: mol_type = other DNA
agctctttctcgattccgtg

SEQ ID NO: 23 - DNA, 20 bp, synthetic construct
  misc_feature 1..20: note = Primer 20
  source 1..20: mol_type = other DNA
gggtagacacaagctgagcc

SEQ ID NO: 24 - DNA, 20 bp, synthetic construct
  misc_feature 1..20: note = Primer 21
  source 1..20: mol_type = other DNA
caactatcaggctccaccac

SEQ ID NO: 25 - DNA, 20 bp, synthetic construct
  misc_feature 1..20: note = Primer 22
  source 1..20: mol_type = other DNA
ctcagacggttttcagggtt

SEQ ID NO: 26 - DNA, 20 bp, synthetic construct
  misc_feature 1..20: note = Primer 23
  source 1..20: mol_type = other DNA
aggctatgggatgatgcaag

SEQ ID NO: 27 - DNA, 20 bp, synthetic construct
  misc_feature 1..20: note = Primer 24
  source 1..20: mol_type = other DNA
gtaggtaggcgatccgttca

SEQ ID NO: 28 - DNA, 20 bp, synthetic construct
  misc_feature 1..20: note = Primer 25
  source 1..20: mol_type = other DNA
cgagaccaacgtgaagggaa

SEQ ID NO: 29 - DNA, 20 bp, synthetic construct
  misc_feature 1..20: note = Primer 26
  source 1..20: mol_type = other DNA
cagacccggacaacgtcttt

SEQ ID NO: 30 - DNA, 20 bp, synthetic construct
  misc_feature 1..20: note = Primer 27
  source 1..20: mol_type = other DNA
ctctggggctcacctacaag

SEQ ID NO: 31 - DNA, 20 bp, synthetic construct
  misc_feature 1..20: note = Primer 28
  source 1..20: mol_type = other DNA
agccctggtgaaatgtaggg

SEQ ID NO: 32 - DNA, 20 bp, synthetic construct
  misc_feature 1..20: note = Primer 29
  source 1..20: mol_type = other DNA
agctctcatctcaaggcaca

SEQ ID NO: 33 - DNA, 20 bp, synthetic construct
  misc_feature 1..20: note = Primer 30
  source 1..20: mol_type = other DNA
ggaaagatccactgcttcca

SEQ ID NO: 34 - DNA, 22 bp, synthetic construct
  misc_feature 1..22: note = Primer 31
  source 1..22: mol_type = other DNA
agcacaggtggtgaacgaacca

SEQ ID NO: 35 - DNA, 23 bp, synthetic construct
  misc_feature 1..23: note = Primer 32
  source 1..23: mol_type = other DNA
tccaggcctcttgatgctaccga

SEQ ID NO: 36 - DNA, 24 bp, synthetic construct
  misc_feature 1..24: note = Primer 34
  source 1..24: mol_type = other DNA
caccgccaaataaggcacgttatc

SEQ ID NO: 37 - DNA, 24 bp, synthetic construct
  misc_feature 1..24: note = Primer 35
  source 1..24: mol_type = other DNA
aaacgataacgtgccttatttggc

SEQ ID NO: 38 - DNA, 25 bp, synthetic construct
  misc_feature 1..25: note = Primer 36
  source 1..25: mol_type = other DNA
caccgaccagataacgtgccttatt

SEQ ID NO: 39 - DNA, 24 bp, synthetic construct
  misc_feature 1..24: note = Primer 37
  source 1..24: mol_type = other DNA
aaacaataaggcacgttatctggt

SEQ ID NO: 40 - DNA, 19 bp, synthetic construct
  misc_feature 1..19: note = Primer 38
  source 1..19: mol_type = other DNA
ttgcagtggttaccgttcg

SEQ ID NO: 41 - DNA, 20 bp, synthetic construct
  misc_feature 1..20: note = Primer 39
  source 1..20: mol_type = other DNA
tagtaggcatcttgtggggg

SEQ ID NO: 42 - DNA, 20 bp, synthetic construct
  misc_feature 1..20: note = Primer 40
  source 1..20: mol_type = other DNA
atgaatggagctataggagg

SEQ ID NO: 43 - DNA, 61 bp, synthetic construct
  misc_feature 1..61: note = Primer 41
  source 1..61: mol_type = other DNA
ccacgtctcctgcttgctttaacagagagaagttcgtggcatcgccatcttccagcaggcg

SEQ ID NO: 44 - DNA, 61 bp, synthetic construct
  misc_feature 1..61: note = Primer 42
  source 1..61: mol_type = other DNA
tgttaaagcaagcaggagacgtggaagaaaaccccggtcctatgaagatggacaaaaagac

SEQ ID NO: 45 - DNA, 21 bp, synthetic construct
  misc_feature 1..21: note = Primer 43
  source 1..21: mol_type = other DNA
ttacagcccggaccgacgatg

SEQ ID NO: 46 - DNA, 32 bp, synthetic construct
  misc_feature 1..32: note = Primer 44
  source 1..32: mol_type = other DNA
atctgacccgggatgaatggagctataggagg

SEQ ID NO: 47 - DNA, 33 bp, synthetic construct
  misc_feature 1..33: note = Primer 45
  source 1..33: mol_type = other DNA
gtagctgctagcttacagcccggaccgacgatg

SEQ ID NO: 48 - DNA, 31 bp, synthetic construct
  misc_feature 1..31: note = Primer 46
  source 1..31: mol_type = other DNA
caggtccccgggatggatcctgatgatgttg

SEQ ID NO: 49 - DNA, 31 bp, synthetic construct
  misc_feature 1..31: note = Primer 47
  source 1..31: mol_type = other DNA
gcatgtgctagcttagagctttaaatctctg

SEQ ID NO: 50 - DNA, 9289 bp, synthetic construct
  misc_feature 1..9289: note = pX330-GFP
  source 1..9289: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 51 - DNA, 9291 bp, synthetic construct
  misc_feature 1..9291: note = CRISPR1
  source 1..9291: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccaaataaggcacgttatcgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 52 - DNA, 9292 bp, synthetic construct
  misc_feature 1..9292: note = CRISPR3
  source 1..9292: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaccagataacgtgccttattgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 53 - DNA, 6061 bp, synthetic construct
  misc_feature 1..6061: note = pJet-Has
  source 1..6061: mol_type = other DNA
gcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagatttttgaatgaagggcctgagggtgggcagtctgtctatcatgtacatctccatattctgggaggtcgtcagttgggctggcctcctggctaagatttttgcaccacaagagatgctgcatgtgtacaaatcactagcaaatagatttgtttcccatcaacttagccactgttaatgtaaattgttcttggatatgtgtctttggagggcaataaatgctctgaacagcacttgcacaataaagatacagcatgtgggaatgatctgtctcatgtgtcttactgatggtattggttctgtaagataaaatattgtgtctgggatgtgtttggctctactattaatggtgctctattgattgtgatttgtcatttgaaacctgaggatgcgactgtatagcagtctttcatgcatttttggaaaaaaacttaagctttttgaaagctgctgctacaactttttgtattgttataaagttttgtattgtttttttaattgtgaaattataaagatgccgtgcagggactgtttgaagcaaagtgcattgttttagaaacctacaactctagttcaagcactccatcagtatctgcttaatctttgtcatcctttgctatgagaaaatattaagcagtagtctaaaggtactatgaaactataacatagctgacattgtatttataactacgtcatgattttgatagaattgaggacttgaaaatgttaaactattcatgtagggcctcttaagatgcttaagttgtttagtaatgtaagtgtgcatttaattgagattttattgggcataatttgtccatcagtatgacactccttgtcagtgttgccttatacttgatgttgttaccggatctctgcaaggcagttattcttgaattaggctcattgaagtgtctgccagtataaatatatagcaactgttctttgtgttaaaattgagaagctaaccagtttttagtgcttctgactgttggaattctttaagcagatgccataagaaaattgtatttgtgatcaccacttctccagagtggttttaacaccaagggcattagagaaagaaaggcaggcgtgtagagaatagtggacagacaaaagctgtgagttacgttatgtttttcagctgaaaagctgtgtttggtaaaagcatatgaaatcactcaacttggaagcattctcttagttctctgatagttctgagcagcagaactcttcacctaagaggttacttcaactggaagactacctagtgcttctgatggcaactatatttaagatgagaataagaggtgtttccagtgtggtagcctcacatctgttgcagtggttaccgttcgtcctcctccgagggacacagcttggccattcactgtggtgacaccaatatgatgatcagcaaatggtgtttattcactactaaacacagcttatatacatttttacctacaaaatcgtgctgtcatgtcccactctgattggttcataccagataacgtgccttatttggccgtttccacattcttttctcatccttcttctcctgttttctctgcatcaaggtcagcacgatagcactgtctctctatgcttagggagaggcctgtcctgtacatcccgtgcccccacaagatgcctactacaacaacatcttctgcatgtcctgcatagcagtgttgggagaatgtgcactacttccactcttctgatttctattttatgtgtttgctttataccagtgttgccatttgggaattaatacatggttgatcaaatcaattgcatcacagctgtatcctgtatcagaggaacattatcaaagcttttgttgctgtatttggtatctgacctgcagataaacatgttttaggaaggttttgcaaaagtagctgtgaaatgagctggtgttgtgatttaacctgacaggcagctaaacagtataccacagagctattcacctactttccctcagtgggaaaagggaagagaactgaggggggggggaataaataagtaaataacaaaataaaactcatggattaagaaaaagactttgtactggaatggatgagaagaataatagtaatgataataatatgtcactctgaaagtaatgcctcttatttctgtggagactacaaacatacaaagagcacaacattccatagagcaaattctcagttacagaatgctattttttttttcaacacagtcaaaatcattaatttttttttgcctgcaatggacaagagctttgaagctgttctcgtaaaaatctgtactagcagaagtgacctgcaatcactactgctgaaatgcacaacccaccacatcattgtgctcacattcactgtttggtttctgtaaatgtacaggaattgtctgaaattagatatgattttttttttctccatgaaggaattcaattacacacctttgcctcatgcacttctttgtcatttttgtcagactgcttctctcctgcaatttgtctcatggcaacaaaatataatggagttctgctgggaacttccctactgccataccactatcatctgcctctgacattttggacaaatgtaataaaataggaggtattactttcagagcagaccttgtatgtatttacaaaacaagtggtacacaaaaaaaattgttcatcccaccaaccaatgcccatcctgtccctgaatagtagctgtcccccacagccttgaccagtttaggtcaacagttctgcttctgtcccctcccagctccttgtaacccctcagccccccttgctggcaggacagtatgagaagctgaaaaactagaatgtcctagttctttgcagtgctgctaatcaacaaccaaaacagtggtgtgttaccaatattgttgatatcacagcatcataccattatgaaggaagtaacccagccaaaatcaggtcagcttgctaacaagagaactgtgcataagtttaagatgtgtgtgttcctcagtaccttaaaaaataagtagtaacgttcaaatgagtagaagagtagaactgagcttaaaacatctgtcagacaacagtgaaccaaccatctttctagaagatctcctacaatattctcagctgccatggaaaatcgatgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattattatccgctcataattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 54 - DNA, 6929 bp, synthetic construct
  misc_feature 1..6929: note = pCAGG-Neo-IRES-GFP
  source 1..6929: mol_type = other DNA
tcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatctagcctccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggg

SEQ ID NO: 55 - DNA, 10461 bp, synthetic construct
  misc_feature 1..10461: note = Targeting Vector
  source 1..10461: mol_type = other DNA
gcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagatttttgaatgaagggcctgagggtgggcagtctgtctatcatgtacatctccatattctgggaggtcgtcagttgggctggcctcctggctaagatttttgcaccacaagagatgctgcatgtgtacaaatcactagcaaatagatttgtttcccatcaacttagccactgttaatgtaaattgttcttggatatgtgtctttggagggcaataaatgctctgaacagcacttgcacaataaagatacagcatgtgggaatgatctgtctcatgtgtcttactgatggtattggttctgtaagataaaatattgtgtctgggatgtgtttggctctactattaatggtgctctattgattgtgatttgtcatttgaaacctgaggatgcgactgtatagcagtctttcatgcatttttggaaaaaaacttaagctttttgaaagctgctgctacaactttttgtattgttataaagttttgtattgtttttttaattgtgaaattataaagatgccgtgcagggactgtttgaagcaaagtgcattgttttagaaacctacaactctagttcaagcactccatcagtatctgcttaatctttgtcatcctttgctatgagaaaatattaagcagtagtctaaaggtactatgaaactataacatagctgacattgtatttataactacgtcatgattttgatagaattgaggacttgaaaatgttaaactattcatgtagggcctcttaagatgcttaagttgtttagtaatgtaagtgtgcatttaattgagattttattgggcataatttgtccatcagtatgacactccttgtcagtgttgccttatacttgatgttgttaccggatctctgcaaggcagttattcttgaattaggctcattgaagtgtctgccagtataaatatatagcaactgttctttgtgttaaaattgagaagctaaccagtttttagtgcttctgactgttggaattctttaagcagatgccataagaaaattgtatttgtgatcaccacttctccagagtggttttaacaccaagggcattagagaaagaaaggcaggcgtgtagagaatagtggacagacaaaagctgtgagttacgttatgtttttcagctgaaaagctgtgtttggtaaaagcatatgaaatcactcaacttggaagcattctcttagttctctgatagttctgagcagcagaactcttcacctaagaggttacttcaactggaagactacctagtgcttctgatggcaactatatttaagatgagaataagaggtgtttccagtgtggtagcctcacatctgttgcagtggttaccgttcgtcctcctccgagggacacagcttggccattcactgtggtgacaccaatatgatgatcagcaaatggtgtttattcactactaaacacagcttatatacatttttacctacaaaatcgtgctgtcatgtcccactctgattggttcacacatttccccgaaaagtgccacctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatctagcctccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgccgtttccacattcttttctcatccttcttctcctgttttctctgcatcaaggtcagcacgatagcactgtctctctatgcttagggagaggcctgtcctgtacatcccgtgcccccacaagatgcctactacaacaacatcttctgcatgtcctgcatagcagtgttgggagaatgtgcactacttccactcttctgatttctattttatgtgtttgctttataccagtgttgccatttgggaattaatacatggttgatcaaatcaattgcatcacagctgtatcctgtatcagaggaacattatcaaagcttttgttgctgtatttggtatctgacctgcagataaacatgttttaggaaggttttgcaaaagtagctgtgaaatgagctggtgttgtgatttaacctgacaggcagctaaacagtataccacagagctattcacctactttccctcagtgggaaaagggaagagaactgaggggggggggaataaataagtaaataacaaaataaaactcatggattaagaaaaagactttgtactggaatggatgagaagaataatagtaatgataataatatgtcactctgaaagtaatgcctcttatttctgtggagactacaaacatacaaagagcacaacattccatagagcaaattctcagttacagaatgctattttttttttcaacacagtcaaaatcattaatttttttttgcctgcaatggacaagagctttgaagctgttctcgtaaaaatctgtactagcagaagtgacctgcaatcactactgctgaaatgcacaacccaccacatcattgtgctcacattcactgtttggtttctgtaaatgtacaggaattgtctgaaattagatatgattttttttttctccatgaaggaattcaattacacacctttgcctcatgcacttctttgtcatttttgtcagactgcttctctcctgcaatttgtctcatggcaacaaaatataatggagttctgctgggaacttccctactgccataccactatcatctgcctctgacattttggacaaatgtaataaaataggaggtattactttcagagcagaccttgtatgtatttacaaaacaagtggtacacaaaaaaaattgttcatcccaccaaccaatgcccatcctgtccctgaatagtagctgtcccccacagccttgaccagtttaggtcaacagttctgcttctgtcccctcccagctccttgtaacccctcagccccccttgctggcaggacagtatgagaagctgaaaaactagaatgtcctagttctttgcagtgctgctaatcaacaaccaaaacagtggtgtgttaccaatattgttgatatcacagcatcataccattatgaaggaagtaacccagccaaaatcaggtcagcttgctaacaagagaactgtgcataagtttaagatgtgtgtgttcctcagtaccttaaaaaataagtagtaacgttcaaatgagtagaagagtagaactgagcttaaaacatctgtcagacaacagtgaaccaaccatctttctagaagatctcctacaatattctcagctgccatggaaaatcgatgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattattatccgctcataattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 56 - DNA, 7679 bp, synthetic construct
  misc_feature 1..7679: note = pmCherry-Cry2-CreN
  source 1..7679: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggataggatcaagcttatcgataccgtcgacctcgagctcgccaccatgaagatggacaaaaagactatagtttggtttagaagagacctaaggattgaggataatcctgcattagcagcagctgctcacgaaggatctgtttttcctgtcttcatttggtgtcctgaagaagaaggacagttttatcctggaagagcttcaagatggtggatgaaacaatcacttgctcacttatctcaatccttgaaggctcttggatctgacctcactttaatcaaaacccacaacacgatttcagcgatcttggattgtatccgcgttaccggtgctacaaaagtcgtctttaaccacctctatgatcctgtttcgttagttcgggaccataccgtaaaggagaagctggtggaacgtgggatctctgtgcaaagctacaatggagatctattgtatgaaccgtgggagatatactgcgaaaagggcaaaccttttacgagtttcaattcttactggaagaaatgcttagatatgtcgattgaatccgttatgcttcctcctccttggcggttgatgccaataactgcagcggctgaagcgatttgggcgtgttcgattgaagaactagggctggagaatgaggccgagaaaccgagcaatgcgttgttaactagagcttggtctccaggatggagcaatgctgataagttactaaatgagttcatcgagaagcagttgatagattatgcaaagaacagcaagaaagttgttgggaattctacttcactactttctccgtatctccatttcggggaaataagcgtcagacacgttttccagtgtgcccggatgaaacaaattatatgggcaagagataagaacagtgaaggagaagaaagtgcagatctttttcttaggggaatcggtttaagagagtattctcggtatatatgtttcaacttcccgtttactcacgagcaatcgttgttgagtcatcttcggtttttcccttgggatgctgatgttgataagttcaaggcctggagacaaggcaggaccggttatccgttggtggatgccggaatgagagagctttgggctaccggatggatgcataacagaataagagtgattgtttcaagctttgctgtgaagtttcttctccttccatggaaatggggaatgaagtatttctgggatacacttttggatgctgatttggaatgtgacatccttggctggcagtatatctctgggagtatccccgatggccacgagcttgatcgcttggacaatcccgcgttacaaggcgccaaatatgacccagaaggtgagtacataaggcaatggcttcccgagcttgcgagattgccaactgaatggatccatcatccatgggacgctcctttaaccgtactcaaagcttctggtgtggaactcggaacaaactatgcgaaacccattgtagacatcgacacagctcgtgagctactagctaaagctatttcaagaacccgtgaagcacagatcatgatcggagcagcacctgatgagattgtagcagatagcttcgaggccttaggggctaataccattaaagaacctggtctttgcccatctgtgtcttctaatgaccaacaagtaccttcggctgttcgttacaacgggtcaaagagagtgaaacctgaggaagaagaagagagagacatgaagaaatctaggggattcgatgaaagggagttgttttcgactgctgaatcttcttcttcttcgagtgtgtttttcgtttcgcagtcttgctcgttggcatcagaagggaagaatctggaaggtattcaagattcatctgatcagattactacaagtttgggaaaaaatggttgcaaaggtggcggtggctctggaggtggtgggtccggaggaggcggccgcacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgtaacccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggac

SEQ ID NO: 57 - DNA, 6655 bp, synthetic construct
  misc_feature 1..6655: note = pmCherry-CIBN-CreC
  misc_feature 1717: note = n is a, c, g, or t
  source 1..6655: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacntaagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggataggatcaagcttatcgataccgtcgacctcgagctcgccaccatgaatggagctataggaggtgaccttttgctcaattttcctgacatgtcggtcctagagcgccaaagggctcacctcaagtacctcaatcccacctttgattctcctctcgccggcttctttgccgattcttcaatgattaccggcggcgagatggacagctatctttcgactgccggtttgaatcttccgatgatgtacggtgagacgacggtggaaggtgattcaagactctcaatttcgccggaaacgacgcttgggactggaaatttcaagaaacggaagtttgatacagagactaaggattgtaatgagaagaagaagaagatgacgatgaacagagatgacctagtagaagaaggagaagaagagaagtcgaaaataacagagcaaaacaatgggagcacaaaaagcatcaagaagatgaaacacaaagccaagaaagaagagaacaatttctctaatgattcatctaaagtgacgaaggaattggagaaaacggattatattcatggtggcggtggctctggaggtggtgggtccggaggaggcggccgccgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagcaccgcctacatacctcgctctgctaatcctgttaccagtggctgctgccagtggcgataagtcgtgtcttaccgggttgga

SEQ ID NO: 58 - DNA, 8968 bp, synthetic construct
  misc_feature 1..8968: note = pB-RAGE-GFP
  source 1..8968: mol_type = other DNA
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcggaattaaccctcactaaagggaacaaaagctggctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccatcaagcttaggatccggaacccttaatataacttcgtataatgtatgctatacgaagttattaggtccctcgacctgcagcccaagcttacttaccatgtcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccctcgagaaaaaaaatataaaagagatggaggaacgggaaaaagttagttgtggtgataggtggcaagtggtattccgtaagaacaacaagaaaagcatttcatattatggctgaactgagcgaacaagtgcaaaatttaagcatcaacgacaacaacgagaatggttatgttcctcctcacttaagaggaaaaccaagaagtgccagaaataacatgagcaactacaataacaacaacggcggctacaacggtggccgtggcggtggcagcttctttagcaacaaccgtcgtggtggttacggcaacggtggtttcttcggtggaaacaacggtggcagcagatctaacggccgttctggtggtagatggatcgatggcaaacatgtcccagctccaagaaacgaaaaggccgagatcgccatatttggtgtccccgaggatccggaacccttaatataacttcgtataatgtatgctatacgaagttattaggtccctcgaagaggttcactagggctagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggagtggggtggggcaggacagcaagggggaggattgggaagacaatagcaggcatgctggggatgcggtgggctctatggcttctgaggcggaaagaaccagctggggcttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 59 - DNA, 6188 bp, synthetic construct
  misc_feature 1..6188: note = pCAGG-IRES-GFP
  source 1..6188: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagcacctgcacatgggacgtcgacctgaggtaattataacccgggccctatatatggatcggctagccgatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattg

SEQ ID NO: 60 - DNA, 9644 bp, synthetic construct
  misc_feature 1..9644: note = pCAGG-Optogenes
  source 1..9644: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagcacctgcacatgggacgtcgacctgaggtaattataaccccccgggatgaatggagctataggaggtgaccttttgctcaattttcctgacatgtcggtcctagagcgccaaagggctcacctcaagtacctcaatcccacctttgattctcctctcgccggcttctttgccgattcttcaatgattaccggcggcgagatggacagctatctttcgactgccggtttgaatcttccgatgatgtacggtgagacgacggtggaaggtgattcaagactctcaatttcgccggaaacgacgcttgggactggaaatttcaagaaacggaagtttgatacagagactaaggattgtaatgagaagaagaagaagatgacgatgaacagagatgacctagtagaagaaggagaagaagagaagtcgaaaataacagagcaaaacaatgggagcacaaaaagcatcaagaagatgaaacacaaagccaagaaagaagagaacaatttctctaatgattcatctaaagtgacgaaggaattggagaaaacggattatattcatggtggcggtggctctggaggtggtgggtccggaggaggcggccgccgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatgccacgaacttctctctgttaaagcaagcaggagacgtggaagaaaaccccggtcctatgaagatggacaaaaagactatagtttggtttagaagagacctaaggattgaggataatcctgcattagcagcagctgctcacgaaggatctgtttttcctgtcttcatttggtgtcctgaagaagaaggacagttttatcctggaagagcttcaagatggtggatgaaacaatcacttgctcacttatctcaatccttgaaggctcttggatctgacctcactttaatcaaaacccacaacacgatttcagcgatcttggattgtatccgcgttaccggtgctacaaaagtcgtctttaaccacctctatgatcctgtttcgttagttcgggaccataccgtaaaggagaagctggtggaacgtgggatctctgtgcaaagctacaatggagatctattgtatgaaccgtgggagatatactgcgaaaagggcaaaccttttacgagtttcaattcttactggaagaaatgcttagatatgtcgattgaatccgttatgcttcctcctccttggcggttgatgccaataactgcagcggctgaagcgatttgggcgtgttcgattgaagaactagggctggagaatgaggccgagaaaccgagcaatgcgttgttaactagagcttggtctccaggatggagcaatgctgataagttactaaatgagttcatcgagaagcagttgatagattatgcaaagaacagcaagaaagttgttgggaattctacttcactactttctccgtatctccatttcggggaaataagcgtcagacacgttttccagtgtgcccggatgaaacaaattatatgggcaagagataagaacagtgaaggagaagaaagtgcagatctttttcttaggggaatcggtttaagagagtattctcggtatatatgtttcaacttcccgtttactcacgagcaatcgttgttgagtcatcttcggtttttcccttgggatgctgatgttgataagttcaaggcctggagacaaggcaggaccggttatccgttggtggatgccggaatgagagagctttgggctaccggatggatgcataacagaataagagtgattgtttcaagctttgctgtgaagtttcttctccttccatggaaatggggaatgaagtatttctgggatacacttttggatgctgatttggaatgtgacatccttggctggcagtatatctctgggagtatccccgatggccacgagcttgatcgcttggacaatcccgcgttacaaggcgccaaatatgacccagaaggtgagtacataaggcaatggcttcccgagcttgcgagattgccaactgaatggatccatcatccatgggacgctcctttaaccgtactcaaagcttctggtgtggaactcggaacaaactatgcgaaacccattgtagacatcgacacagctcgtgagctactagctaaagctatttcaagaacccgtgaagcacagatcatgatcggagcagcacctgatgagattgtagcagatagcttcgaggccttaggggctaataccattaaagaacctggtctttgcccatctgtgtcttctaatgaccaacaagtaccttcggctgttcgttacaacgggtcaaagagagtgaaacctgaggaagaagaagagagagacatgaagaaatctaggggattcgatgaaagggagttgttttcgactgctgaatcttcttcttcttcgagtgtgtttttcgtttcgcagtcttgctcgttggcatcagaagggaagaatctggaaggtattcaagattcatctgatcagattactacaagtttgggaaaaaatggttgcaaaggtggcggtggctctggaggtggtgggtccggaggaggcggccgcacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgtaagctagcctccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattg

SEQ ID NO: 61 - DNA, 9166 bp, synthetic construct
  misc_feature 1..9166: note = pB-RAGE-mCherry
  source 1..9166: mol_type = other DNA
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcggaattaaccctcactaaagggaacaaaagctggctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccatcaagcttaggatccggaacccttaatataacttcgtataatgtatgctatacgaagttattaggtccctcgacctgcagcccaagcttacttaccatgtcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccctcgagaaaaaaaatataaaagagatggaggaacgggaaaaagttagttgtggtgataggtggcaagtggtattccgtaagaacaacaagaaaagcatttcatattatggctgaactgagcgaacaagtgcaaaatttaagcatcaacgacaacaacgagaatggttatgttcctcctcacttaagaggaaaaccaagaagtgccagaaataacatgagcaactacaataacaacaacggcggctacaacggtggccgtggcggtggcagcttctttagcaacaaccgtcgtggtggttacggcaacggtggtttcttcggtggaaacaacggtggcagcagatctaacggccgttctggtggtagatggatcgatggcaaacatgtcccagctccaagaaacgaaaaggccgagatcgccatatttggtgtccccgaggatccggaacccttaatataacttcgtataatgtatgctatacgaagttattaggtccctcgaagaggttcactagggctagcagttataggatctccgccaccatgctgtgctgcatcagaagaactaaaccggttgagaagaatgaagaggccgatcaggagctgcagtcgacggtaccgcgggcccgggatccaccggtagcatccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggcagtggagagggcagaggaagtctgctaacatgcggtgacgtcgaggagaatcctggcccaactagtgtttaaacgcggccgcggcaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggagtggggtggggcaggacagcaagggggaggattgggaagacaatagcaggcatgctggggatgcggtgggctctatggcttctgaggcggaaagaaccagctggggcttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 62 - DNA, 4526 bp, synthetic construct
  misc_feature 1..4526: note = pSK BS-PGK-DTA
  source 1..4526: mol_type = other DNA
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatccccaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcaggtcctcgccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcagatcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcag

SEQ ID NO: 63 - DNA, 4929 bp, synthetic construct
  misc_feature 1..4929: note = pGK-IRES-GFP
  source 1..4929: mol_type = other DNA
tcgacaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgccccgggccctatatatggatcggctagccgatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggg

SEQ ID NO: 64 - DNA, 5568 bp, synthetic construct
  misc_feature 1..5568: note = pGK-DTA-IRES-GFP
  source 1..5568: mol_type = other DNA
tcgacaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgccccgggatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaagctagcctccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggg

SEQ ID NO: 65 - DNA, 450 bp, synthetic construct
  misc_feature 1..450: note = Negative Mag recombinase
  source 1..450: mol_type = other DNA
catactctttatgcccccggtggatatgacattatgggatatctggaccagatcggcaaccggccaaacccgcaggtggaactgggccccgtggatacatcctgcgccttgattctttgtgacctgaaacagaaagacaccccgatagtttacgcgagtgaagccttcctctacatgacaggttacagcaacgcagaggtgctgggccggaattgccggtttctgcaaagccctgacggcatggtgaagcccaagagcacccggaagtacgtggatagtaacacaatcaatactatgcgcaaggcaatcgacaggaatgccgaggtgcaggttgaagtagtcaattttaaaaagaatggacagcgatttgttaatttcctgactatgatacctgttagggacgaaacaggcgagtatcgatactctatgggattccagtgcgaaacagaa

SEQ ID NO: 66 - DNA, 20 bp, synthetic construct
  misc_feature 1..20: note = Guide 1
  source 1..20: mol_type = other DNA
gccaaataaggcacgttatc

SEQ ID NO: 67 - DNA, 20 bp, synthetic construct
  misc_feature 1..20: note = Guide 2
  source 1..20: mol_type = other DNA
aatgtggaaacggccaaata

SEQ ID NO: 68 - DNA, 20 bp, synthetic construct
  misc_feature 1..20: note = Guide 3
  source 1..20: mol_type = other DNA
accagataacgtgccttatt

SEQ ID NO: 69 - DNA, 20 bp, synthetic construct
  misc_feature 1..20: note = Guide 4
  source 1..20: mol_type = other DNA
acatgacagcacgattttgt

SEQ ID NO: 70 - DNA, 20 bp, synthetic construct
  misc_feature 1..20: note = Guide 5
  source 1..20: mol_type = other DNA
ctggtatgaaccaatcagag

SEQ ID NO: 71 - DNA, 20 bp, synthetic construct
  misc_feature 1..20: note = Guide 6
  source 1..20: mol_type = other DNA
tggtatgaaccaatcagagt

SEQ ID NO: 72 - DNA, 20 bp, synthetic construct
  misc_feature 1..20: note = Guide 7
  source 1..20: mol_type = other DNA
gaccttgatgcagagaaaac

SEQ ID NO: 73 - DNA, 20 bp, synthetic construct
  misc_feature 1..20: note = Guide 8
  source 1..20: mol_type = other DNA
ctcctgttttctctgcatca

SEQ ID NO: 74 - DNA, 20 bp, synthetic construct
  misc_feature 1..20: note = Guide 9
  source 1..20: mol_type = other DNA
gcagagaaaacaggagaaga

SEQ ID NO: 75 - DNA, 20 bp, synthetic construct
  misc_feature 1..20: note = Guide 10
  source 1..20: mol_type = other DNA
agaaggatgagaaaagaatg

SEQ ID NO: 76 - DNA, 20 bp, synthetic construct
  misc_feature 1..20: note = Guide 11
  source 1..20: mol_type = other DNA
ctgtcatgtcccactctgat

SEQ ID NO: 77 - DNA, 20 bp, synthetic construct
  misc_feature 1..20: note = Guide 12
  source 1..20: mol_type = other DNA
atgagaaaagaatgtggaaa

SEQ ID NO: 78 - DNA, 23 bp, synthetic construct
  misc_feature 1..23: note = Guide 1 potential off target
  source 1..23: mol_type = other DNA
ccaacagaaggcacgttatccag

SEQ ID NO: 79 - DNA, 23 bp, synthetic construct
  misc_feature 1..23: note = Guide 1 potential off target
  source 1..23: mol_type = other DNA
tcaaaataaagtacgttatctag

SEQ ID NO: 80 - DNA, 23 bp, synthetic construct
  misc_feature 1..23: note = Guide 1 potential off target
  source 1..23: mol_type = other DNA
ggcatataaagcacgttatacag

SEQ ID NO: 81 - DNA, 23 bp, synthetic construct
  misc_feature 1..23: note = Guide 1 potential off target
  source 1..23: mol_type = other DNA
gcataataatgtacgttatctgg

SEQ ID NO: 82 - DNA, 23 bp, synthetic construct
  misc_feature 1..23: note = Guide 1 potential off target
  source 1..23: mol_type = other DNA
actaaatcaggcacgtgatctgg

SEQ ID NO: 83 - DNA, 23 bp, synthetic construct
  misc_feature 1..23: note = Guide 1 potential off target
  source 1..23: mol_type = other DNA
gctaaattaagctcgttatcggg

SEQ ID NO: 84 - DNA, 23 bp, synthetic construct
  misc_feature 1..23: note = Guide 1 potential off target
  source 1..23: mol_type = other DNA
gtcaaatgaggcatgttatcagg

SEQ ID NO: 85 - DNA, 23 bp, synthetic construct
  misc_feature 1..23: note = Guide 1 potential off target
  source 1..23: mol_type = other DNA
ttcaaataagccacgttattcag

SEQ ID NO: 86 - DNA, 23 bp, synthetic construct
  misc_feature 1..23: note = Guide 1 potential off target
  source 1..23: mol_type = other DNA
gtcaaacaaggcatgttatcagg

SEQ ID NO: 87 - DNA, 23 bp, synthetic construct
  misc_feature 1..23: note = Guide 1 potential off target
  source 1..23: mol_type = other DNA
ccctaataaagcacgttttcagg

SEQ ID NO: 88 - DNA, 22 bp, synthetic construct
  misc_feature 1..22: note = FWD primer
  source 1..22: mol_type = other DNA
accatcttcgggaagattatcc

SEQ ID NO: 89 - DNA, 22 bp, synthetic construct
  misc_feature 1..22: note = REV primer
  source 1..22: mol_type = other DNA
cttctgcttcagataacctgac

SEQ ID NO: 90 - DNA, 20 bp, synthetic construct
  misc_feature 1..20: note = GAPDH primers: Forward
  source 1..20: mol_type = other DNA
actgtcaaggctgagaacgg

SEQ ID NO: 91 - DNA, 20 bp, synthetic construct
  misc_feature 1..20: note = GAPDH primers: Reverse
  source 1..20: mol_type = other DNA
acctgcatctgcccatttga

SEQ ID NO: 92 - DNA, 657 bp, Corynebacterium diphtheriae
  source 1..657: mol_type = other DNA
atggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaa

SEQ ID NO: 93 - protein, 218 aa, Corynebacterium diphtheriae
MDPDDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSLCEGTLLLWCDIIGQTTYRDLKL

SEQ ID NO: 94 - DNA, 852 bp, Gallus gallus
  source 1..852: mol_type = other DNA
atgatgacagacataaaagatggaccacgctcaggggaagatgtatcagatgcaagatctttccctggttccaaaggaatgaacttacctgctagcaagtctgtggactctggaattctgcctgatgacagttacagaatggattatccagagataggagtatgtgttataataaacaataagaacttccaccgagataccggactgtcatctcgttcaggcacggatgcagatgctgcaagtgtcagagaagtttttatgaagctgggatataaagtcaagcttaacaatgatctgtcaagcagagatatttttaagctattgaaaaatgtttctgaagaagatcacagcaagcgaagcagttttgtttgtgtgttgctaagccatggcgatgaaggactcttctatggtacagatggccctcttgaactgaaagtactaaccagccttttcagaggtgacaagtgcagaagtctagcagggaaacccaaactctttttcattcaggcctgtagaggaacagaattagattctggtattgaagcagacagtggaccagatgaaacagtgtgtcaaaaaatacctgtagaagcagacttcctgtatgcatattctactgctccaggctactactcctggaggaacgcagctgaaggctcctggtttattcagtctctgtgtaggatgctgaaggaacacgccaggaaacttgaactcatgcagattttaactcgtgtaaatcgcagagtggcagaatatgaatcctgctccactcgacaggatttcaatgcaaagaaacagattccatgcattgtgtctatgcttaccaaagaattctactttccttgctaa

SEQ ID NO: 95 - protein, 283 aa, Gallus gallus
MMTDIKDGPRSGEDVSDARSFPGSKGMNLPASKSVDSGILPDDSYRMDYPEIGVCVIINNKNFHRDTGLSSRSGTDADAASVREVFMKLGYKVKLNNDLSSRDIFKLLKNVSEEDHSKRSSFVCVLLSHGDEGLFYGTDGPLELKVLTSLFRGDKCRSLAGKPKLFFIQACRGTELDSGIEADSGPDETVCQKIPVEADFLYAYSTAPGYYSWRNAAEGSWFIQSLCRMLKEHARKLELMQILTRVNRRVAEYESCSTRQDFNAKKQIPCIVSMLTKEFYFPC

SEQ ID NO: 96 - DNA, 852 bp, Gallus gallus
  source 1..852: mol_type = other DNA
atgatgacagacataaaagatggaccacgctcaggggaagatgtatcagctgcaagatctttccctggttccaaaggaatgaacttacctgctagcaagtctgtggcctctggaattctgcctgatgacagttacagaatggattatccagagataggagtatgtgttataataaacaataagaacttccaccgagataccggactgtcatctcgttcaggcacggatgcagatgctgcaagtgtcagagaagtttttatgaagctgggatataaagtcaagcttaacaatgatctgtcaagcagagatatttttaagctattgaaaaatgtttctgaagaagatcacagcaagcgaagcagttttgtttgtgtgttgctaagccatggcgatgaaggactcttctatggtacagatggccctcttgaactgaaagtactaaccagccttttcagaggtgacaagtgcagaagtctagcagggaaacccaaactctttttcattcaggcctgtagaggaacagaattagattctggtattgaagcagccagtggaccagatgaaacagtgtgtcaaaaaatacctgtagaagcagacttcctgtatgcatattctactgctccaggctactactcctggaggaacgcagctgaaggctcctggtttattcagtctctgtgtaggatgctgaaggaacacgccaggaaacttgaactcatgcagattttaactcgtgtaaatcgcagagtggcagaatatgaatcctgctccactcgacaggatttcaatgcaaagaaacagattccatgcattgagtctatgcttaccaaagaattctactttccttgctaa

SEQ ID NO: 97 - protein, 283 aa, Gallus gallus
MMTDIKDGPRSGEDVSAARSFPGSKGMNLPASKSVASGILPDDSYRMDYPEIGVCVIINNKNFHRDTGLSSRSGTDADAASVREVFMKLGYKVKLNNDLSSRDIFKLLKNVSEEDHSKRSSFVCVLLSHGDEGLFYGTDGPLELKVLTSLFRGDKCRSLAGKPKLFFIQACRGTELDSGIEAASGPDETVCQKIPVEADFLYAYSTAPGYYSWRNAAEGSWFIQSLCRMLKEHARKLELMQILTRVNRRVAEYESCSTRQDFNAKKQIPCIESMLTKEFYFPC

SEQ ID NO: 98 - DNA, 672 bp, synthetic construct
  misc_feature 1..672: note = Noggin Nucleotide
  source 1..672: mol_type = other DNA
atggatcattcccagtgccttgtgactatatacgccgcggcggtgctgctggggctccggctgcagcagggctcctgccagcactacctgcacatccgcccggctcccagcgacaacctgcccctggtggatctaatcgagcacccggaccctatctttgaccccaaggagaaggatcttaacgagaccttgctaaggagcctcatgggaggacacttcgaccctaactttatggctatgtccctgcccgaggaccggctcggggtagacgatctggccgagctggacttgctgctgcggcagagaccctcgggagcgatgcccggcgaaatcaaggggctggagttctacgacgggctgcagccgggcaagaagcacaggctgagcaagaagctgcgcaggaagctgcagatgtggctctggtcccagaccttctgcccggtcctatacacgtggaacgatctcggcagccgcttttggccccggtacgtcaaagtgggcagctgctacagtaaaaggtcttgctctgtcccagaaggcatggtctgcaaacctgccaagtccgtgcatttaacgatcctgaggtggcggtgccagcggcggggcgggcagcggtgcacgtggatccccatccagtaccccatcatcgcggagtgcaagtgctcctgctag

SEQ ID NO: 99 - protein, 223 aa, synthetic construct
  REGION 1..223: note = Noggin Aminoacid
MDHSQCLVTIYAAAVLLGLRLQQGSCQHYLHIRPAPSDNLPLVDLIEHPDPIFDPKEKDLNETLLRSLMGGHFDPNFMAMSLPEDRLGVDDLAELDLLLRQRPSGAMPGEIKGLEFYDGLQPGKKHRLSKKLRRKLQMWLWSQTFCPVLYTWNDLGSRFWPRYVKVGSCYSKRSCSVPEGMVCKPAKSVHLTILRWRCQRRGGQRCTWIPIQYPIIAECKCSC

SEQ ID NO: 100 - DNA, 1686 bp, synthetic construct
  misc_feature 1..1686: note = pCAGG promotor
  source 1..1686: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggtta

SEQ ID NO: 101 - DNA, 1941 bp, synthetic construct
  misc_feature 1..1941: note = Optogen 1 - NLS-Cry2-delta535-L348F-Cre-N (AA 19-104)
  source 1..1941: mol_type = other DNA
atgcctaaaaagaagcgtaaagtcatgaagatgaagatggacaaaaagactatagtttggtttagaagagacctaaggattgaggataatcctgcattagcagcagctgctcacgaaggatctgtttttcctgtcttcatttggtgtcctgaagaagaaggacagttttatcctggaagagcttcaagatggtggatgaaacaatcacttgctcacttatctcaatccttgaaggctcttggatctgacctcactttaatcaaaacccacaacacgatttcagcgatcttggattgtatccgcgttaccggtgctacaaaagtcgtctttaaccacctctatgatcctgtttcgttagttcgggaccataccgtaaaggagaagctggtggaacgtgggatctctgtgcaaagctacaatggagatctattgtatgaaccgtgggagatatactgcgaaaagggcaaaccttttacgagtttcaattcttactggaagaaatgcttagatatgtcgattgaatccgttatgcttcctcctccttggcggttgatgccaataactgcagcggctgaagcgatttgggcgtgttcgattgaagaactagggctggagaatgaggccgagaaaccgagcaatgcgttgttaactagagcttggtctccaggatggagcaatgctgataagttactaaatgagttcatcgagaagcagttgatagattatgcaaagaacagcaagaaagttgttgggaattctacttcactactttctccgtatctccatttcggggaaataagcgtcagacacgttttccagtgtgcccggatgaaacaaattatatgggcaagagataagaacagtgaaggagaagaaagtgcagatctttttcttaggggaatcggtttaagagagtattctcggtatatatgtttcaacttcccgtttactcacgagcaatcgttgttgagtcatcttcggtttttcccttgggatgctgatgttgataagttcaaggcctggagacaaggcaggaccggttatccgttggtggatgccggaatgagagagttttgggctaccggatggatgcataacagaataagagtgattgtttcaagctttgctgtgaagtttcttctccttccatggaaatggggaatgaagtatttctgggatacacttttggatgctgatttggaatgtgacatccttggctggcagtatatctctgggagtatccccgatggccacgagcttgatcgcttggacaatcccgcgttacaaggcgccaaatatgacccagaaggtgagtacataaggcaatggcttcccgagcttgcgagattgccaactgaatggatccatcatccatgggacgctcctttaaccgtactcaaagcttctggtgtggaactcggaacaaactatgcgaaacccattgtagacatcgacacagctcgtgagctactagctaaagctatttcaagaacccgtgaagcacagatcatgatcggagcagcacctgatgagattgtagcagatagcttcgaggccttaggggctaataccattaaagaacctggtctttgcccatctgtgtcttctaatgaccaacaagtaccttcggctgttggtggcggtggctctggaggtggtgggtccggaggaggcggccgcacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgtaa

SEQ ID NO: 102 - DNA, 1275 bp, synthetic construct
  misc_feature 1..1275: note = Optogen 2 - CIBN(aa1-170)-NLS-Cre-C(aa106-343)
  source 1..1275: mol_type = other DNA
atgaatggagctataggaggtgaccttttgctcaattttcctgacatgtcggtcctagagcgccaaagggctcacctcaagtacctcaatcccacctttgattctcctctcgccggcttctttgccgattcttcaatgattaccggcggcgagatggacagctatctttcgactgccggtttgaatcttccgatgatgtacggtgagacgacggtggaaggtgattcaagactctcaatttcgccggaaacgacgcttgggactggaaatttcaagaaacggaagtttgatacagagactaaggattgtaatgagaagaagaagaagatgacgatgaacagagatgacctagtagaagaaggagaagaagagaagtcgaaaataacagagcaaaacaatgggagcacaaaaagcatcaagaagatgaaacacaaagccaagaaagaagagaacaatttctctaatgattcatctaaagtgacgaaggaattggagaaaacggattatattcatggtggcggtggctctggaggtggtgggtccggaggaggcggccgccgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 103 - DNA, 585 bp, synthetic construct
  misc_feature 1..585: note = IRES sequence
  source 1..585: mol_type = other DNA
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccaca

SEQ ID NO: 104 - DNA, 524 bp, synthetic construct
  misc_feature 1..524: note = polyadenylation site sequence (Rabbit beta-globin)
  source 1..524: mol_type = other DNA
actcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 105 - DNA, 1496 bp, synthetic construct
  misc_feature 1..1496: note = 5 homology arm
  source 1..1496: mol_type = other DNA
gaatgaagggcctgagggtgggcagtctgtctatcatgtacatctccatattctgggaggtcgtcagttgggctggcctcctggctaagatttttgcaccacaagagatgctgcatgtgtacaaatcactagcaaatagatttgtttcccatcaacttagccactgttaatgtaaattgttcttggatatgtgtctttggagggcaataaatgctctgaacagcacttgcacaataaagatacagcatgtgggaatgatctgtctcatgtgtcttactgatggtattggttctgtaagataaaatattgtgtctgggatgtgtttggctctactattaatggtgctctattgattgtgatttgtcatttgaaacctgaggatgcgactgtatagcagtctttcatgcatttttggaaaaaaacttaagctttttgaaagctgctgctacaactttttgtattgttataaagttttgtattgtttttttaattgtgaaattataaagatgccgtgcagggactgtttgaagcaaagtgcattgttttagaaacctacaactctagttcaagcactccatcagtatctgcttaatctttgtcatcctttgctatgagaaaatattaagcagtagtctaaaggtactatgaaactataacatagctgacattgtatttataactacgtcatgattttgatagaattgaggacttgaaaatgttaaactattcatgtagggcctcttaagatgcttaagttgtttagtaatgtaagtgtgcatttaattgagattttattgggcataatttgtccatcagtatgacactccttgtcagtgttgccttatacttgatgttgttaccggatctctgcaaggcagttattcttgaattaggctcattgaagtgtctgccagtataaatatatagcaactgttctttgtgttaaaattgagaagctaaccagtttttagtgcttctgactgttggaattctttaagcagatgccataagaaaattgtatttgtgatcaccacttctccagagtggttttaacaccaagggcattagagaaagaaaggcaggcgtgtagagaatagtggacagacaaaagctgtgagttacgttatgtttttcagctgaaaagctgtgtttggtaaaagcatatgaaatcactcaacttggaagcattctcttagttctctgatagttctgagcagcagaactcttcacctaagaggttacttcaactggaagactacctagtgcttctgatggcaactatatttaagatgagaataagaggtgtttccagtgtggtagcctcacatctgttgcagtggttaccgttcgtcctcctccgagggacacagcttggccattcactgtggtgacaccaatatgatgatcagcaaatggtgtttattcactactaaacacagcttatatacatttttacctacaaaatcgtgctgtcatgtcccactctgattggttca

SEQ ID NO: 106 - DNA, 1564 bp, synthetic construct
  misc_feature 1..1564: note = 3 homology arm
  source 1..1564: mol_type = other DNA
gccgtttccacattcttttctcatccttcttctcctgttttctctgcatcaaggtcagcacgatagcactgtctctctatgcttagggagaggcctgtcctgtacatcccgtgcccccacaagatgcctactacaacaacatcttctgcatgtcctgcatagcagtgttgggagaatgtgcactacttccactcttctgatttctattttatgtgtttgctttataccagtgttgccatttgggaattaatacatggttgatcaaatcaattgcatcacagctgtatcctgtatcagaggaacattatcaaagcttttgttgctgtatttggtatctgacctgcagataaacatgttttaggaaggttttgcaaaagtagctgtgaaatgagctggtgttgtgatttaacctgacaggcagctaaacagtataccacagagctattcacctactttccctcagtgggaaaagggaagagaactgaggggggggggaataaataagtaaataacaaaataaaactcatggattaagaaaaagactttgtactggaatggatgagaagaataatagtaatgataataatatgtcactctgaaagtaatgcctcttatttctgtggagactacaaacatacaaagagcacaacattccatagagcaaattctcagttacagaatgctattttttttttcaacacagtcaaaatcattaatttttttttgcctgcaatggacaagagctttgaagctgttctcgtaaaaatctgtactagcagaagtgacctgcaatcactactgctgaaatgcacaacccaccacatcattgtgctcacattcactgtttggtttctgtaaatgtacaggaattgtctgaaattagatatgattttttttttctccatgaaggaattcaattacacacctttgcctcatgcacttctttgtcatttttgtcagactgcttctctcctgcaatttgtctcatggcaacaaaatataatggagttctgctgggaacttccctactgccataccactatcatctgcctctgacattttggacaaatgtaataaaataggaggtattactttcagagcagaccttgtatgtatttacaaaacaagtggtacacaaaaaaaattgttcatcccaccaaccaatgcccatcctgtccctgaatagtagctgtcccccacagccttgaccagtttaggtcaacagttctgcttctgtcccctcccagctccttgtaacccctcagccccccttgctggcaggacagtatgagaagctgaaaaactagaatgtcctagttctttgcagtgctgctaatcaacaaccaaaacagtggtgtgttaccaatattgttgatatcacagcatcataccattatgaaggaagtaacccagccaaaatcaggtcagcttgctaacaagagaactgtgcataagtttaagatgtgtgtgttcctcagtaccttaaaaaataagtagtaacgttcaaatgagtagaagagtagaactgagcttaaaacatctgtcagacaacagtgaaccaacc

SEQ ID NO: 107 - DNA, 627 bp, synthetic construct
  misc_feature 1..627: note = Cre-N(aa18-59)_N-Mag_NLS
  source 1..627: mol_type = other DNA
atggccacctctgatgaagtcaggaagaacctgatggacatgttcagggacaggcaggccttctctgaacacacctggaagatgctcctgtctgtgtgcagatcctgggctgcctggtgcaagctgaacggtacccatactctttatgcccccggtggatatgacattatgggatatctggaccagatcggcaaccggccaaacccgcaggtggaactgggccccgtggatacatcctgcgccttgattctttgtgacctgaaacagaaagacaccccgatagtttacgcgagtgaagccttcctctacatgacaggttacagcaacgcagaggtgctgggccggaattgccggtttctgcaaagccctgacggcatggtgaagcccaagagcacccggaagtacgtggatagtaacacaatcaatactatgcgcaaggcaatcgacaggaatgccgaggtgcaggttgaagtagtcaattttaaaaagaatggacagcgatttgttaatttcctgactatgatacctgttagggacgaaacaggcgagtatcgatactctatgggattccagtgcgaaacagaaggcggaagcggtggcgtgcccaagaagaagaggaaagtctag

SEQ ID NO: 108 - DNA, 1344 bp, synthetic construct
  misc_feature 1..1344: note = NLS_P-Mag_Cre-C(aa60-237)
  source 1..1344: mol_type = other DNA
atggtgcccaagaagaagaggaaagtcggcggacatactctttatgcccccggtggatatgacattatgggatatctgaggcagatcaggaaccggccaaacccgcaggtggaactgggccccgtggatacatcctgcgccttgattctttgtgacctgaaacagaaagacaccccgatagtttacgcgagtgaagccttcctctacatgacaggttacagcaacgcagaggtgctgggccggaattgccggtttctgcaaagccctgacggcatggtgaagcccaagagcacccggaagtacgtggatagtaacacaatcaatactatgcgcaaggcaatcgacaggaatgccgaggtgcaggttgaagtagtcaattttaaaaagaatggacagcgatttgttaatttcctgactatgatacctgttagggacgaaacaggcgagtatcgatactctatgggattccagtgcgaaacagaaggtaccaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaagatggggactag

SEQ ID NO: 109 - DNA, 509 bp, synthetic construct
  misc_feature 1..509: note = pGK promotor
  source 1..509: mol_type = other DNA
accgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgc

SEQ ID NO: 110 - DNA, 508 bp, synthetic construct
  misc_feature 1..508: note = pCMV promotor
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 111 - DNA, 448 bp, synthetic construct
  misc_feature 1..448: note = phSyn promotor
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 112 - DNA, 1189 bp, synthetic construct
  misc_feature 1..1189: note = pEFl-a promotor
  source 1..1189: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgag

SEQ ID NO: 113 - DNA, 1056 bp, synthetic construct
  misc_feature 1..1056: note = Cre recombinase
  source 1..1056: mol_type = other DNA
atggcacccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 114 - DNA, 450 bp, synthetic construct
  misc_feature 1..450: note = Positive Mag recombinase
  source 1..450: mol_type = other DNA
catactctttatgcccccggtggatatgacattatgggatatctgaggcagatcaggaaccggccaaacccgcaggtggaactgggccccgtggatacatcctgcgccttgattctttgtgacctgaaacagaaagacaccccgatagtttacgcgagtgaagccttcctctacatgacaggttacagcaacgcagaggtgctgggccggaattgccggtttctgcaaagccctgacggcatggtgaagcccaagagcacccggaagtacgtggatagtaacacaatcaatactatgcgcaaggcaatcgacaggaatgccgaggtgcaggttgaagtagtcaattttaaaaagaatggacagcgatttgttaatttcctgactatgatacctgttagggacgaaacaggcgagtatcgatactctatgggattccagtgcgaaacagaa

SEQ ID NO: 115 - DNA, 720 bp, synthetic construct
  misc_feature 1..720: note = green fluorescence protein (GFP)
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 116 - DNA, 34 bp, synthetic construct
  misc_feature 1..34: note = Lox site
  source 1..34: mol_type = other DNA
ataacttcgtataatgtatgctatacgaagttat

SEQ ID NO: 117 - DNA, 258 bp, synthetic construct
  misc_feature 1..258: note = CreN (AA 19-104)
  source 1..258: mol_type = other DNA
acgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctg

SEQ ID NO: 118 - DNA, 717 bp, synthetic construct
  misc_feature 1..717: note = CreC (AA 106-343)
  source 1..717: mol_type = other DNA
cgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 119 - DNA, 711 bp, synthetic construct
  misc_feature 1..711: note = mCherry/RFP
  source 1..711: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 120 - DNA, 9811 bp, synthetic construct
  misc_feature 1..9811: note = Targeting Vector TV1
  source 1..9811: mol_type = other DNA
gaatgaagggcctgagggtgggcagtctgtctatcatgtacatctccatattctgggaggtcgtcagttgggctggcctcctggctaagatttttgcaccacaagagatgctgcatgtgtacaaatcactagcaaatagatttgtttcccatcaacttagccactgttaatgtaaattgttcttggatatgtgtctttggagggcaataaatgctctgaacagcacttgcacaataaagatacagcatgtgggaatgatctgtctcatgtgtcttactgatggtattggttctgtaagataaaatattgtgtctgggatgtgtttggctctactattaatggtgctctattgattgtgatttgtcatttgaaacctgaggatgcgactgtatagcagtctttcatgcatttttggaaaaaaacttaagctttttgaaagctgctgctacaactttttgtattgttataaagttttgtattgtttttttaattgtgaaattataaagatgccgtgcagggactgtttgaagcaaagtgcattgttttagaaacctacaactctagttcaagcactccatcagtatctgcttaatctttgtcatcctttgctatgagaaaatattaagcagtagtctaaaggtactatgaaactataacatagctgacattgtatttataactacgtcatgattttgatagaattgaggacttgaaaatgttaaactattcatgtagggcctcttaagatgcttaagttgtttagtaatgtaagtgtgcatttaattgagattttattgggcataatttgtccatcagtatgacactccttgtcagtgttgccttatacttgatgttgttaccggatctctgcaaggcagttattcttgaattaggctcattgaagtgtctgccagtataaatatatagcaactgttctttgtgttaaaattgagaagctaaccagtttttagtgcttctgactgttggaattctttaagcagatgccataagaaaattgtatttgtgatcaccacttctccagagtggttttaacaccaagggcattagagaaagaaaggcaggcgtgtagagaatagtggacagacaaaagctgtgagttacgttatgtttttcagctgaaaagctgtgtttggtaaaagcatatgaaatcactcaacttggaagcattctcttagttctctgatagttctgagcagcagaactcttcacctaagaggttacttcaactggaagactacctagtgcttctgatggcaactatatttaagatgagaataagaggtgtttccagtgtggtagcctcacatctgttgcagtggttaccgttcgtcctcctccgagggacacagcttggccattcactgtggtgacaccaatatgatgatcagcaaatggtgtttattcactactaaacacagcttatatacatttttacctacaaaatcgtgctgtcatgtcccactctgattggttcacacatttccccgaaaagtgccacctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttaataacttcgtataatgtatgctatacgaagttataccggtgaagttcctattccgaagttcctattctctagaaagtataggaacttcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcagcggcgccaccaacttcagcctgctgaagcaggccggcgacgtggaggagaaccccggccccgctagctaaagcggccgccaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagaagttcctattccgaagttcctattctctagaaagtataggaacttcgctagcaccggtgccaccatggccacctctgatgaagtcaggaagaacctgatggacatgttcagggacaggcaggccttctctgaacacacctggaagatgctcctgtctgtgtgcagatcctgggctgcctggtgcaagctgaacggtacccatactctttatgcccccggtggatatgacattatgggatatctggaccagatcggcaaccggccaaacccgcaggtggaactgggccccgtggatacatcctgcgccttgattctttgtgacctgaaacagaaagacaccccgatagtttacgcgagtgaagccttcctctacatgacaggttacagcaacgcagaggtgctgggccggaattgccggtttctgcaaagccctgacggcatggtgaagcccaagagcacccggaagtacgtggatagtaacacaatcaatactatgcgcaaggcaatcgacaggaatgccgaggtgcaggttgaagtagtcaattttaaaaagaatggacagcgatttgttaatttcctgactatgatacctgttagggacgaaacaggcgagtatcgatactctatgggattccagtgcgaaacagaaggcggaagcggtggcgtgcccaagaagaagaggaaagtcttcgaaggcagcggcgccaccaacttcagcctgctgaagcaggccggcgacgtggaggagaaccccggccccttcgaagtgcccaagaagaagaggaaagtcggcggacatactctttatgcccccggtggatatgacattatgggatatctgaggcagatcaggaaccggccaaacccgcaggtggaactgggccccgtggatacatcctgcgccttgattctttgtgacctgaaacagaaagacaccccgatagtttacgcgagtgaagccttcctctacatgacaggttacagcaacgcagaggtgctgggccggaattgccggtttctgcaaagccctgacggcatggtgaagcccaagagcacccggaagtacgtggatagtaacacaatcaatactatgcgcaaggcaatcgacaggaatgccgaggtgcaggttgaagtagtcaattttaaaaagaatggacagcgatttgttaatttcctgactatgatacctgttagggacgaaacaggcgagtatcgatactctatgggattccagtgcgaaacagaaggtaccaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaagatggggactaattaattaactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttatacgcgtgccaccatggatcattcccagtgccttgtgactatatacgccgcggcggtgctgctggggctccggctgcagcagggctcctgccagcactacctgcacatccgcccggctcccagcgacaacctgcccctggtggatctaatcgagcacccggaccctatctttgaccccaaggagaaggatcttaacgagaccttgctaaggagcctcatgggaggacacttcgaccctaactttatggctatgtccctgcccgaggaccggctcggggtagacgatctggccgagctggacttgctgctgcggcagagaccctcgggagcgatgcccggcgaaatcaaggggctggagttctacgacgggctgcagccgggcaagaagcacaggctgagcaagaagctgcgcaggaagctgcagatgtggctctggtcccagaccttctgcccggtcctatacacgtggaacgatctcggcagccgcttttggccccggtacgtcaaagtgggcagctgctacagtaaaaggtcttgctctgtcccagaaggcatggtctgcaaacctgccaagtccgtgcatttaacgatcctgaggtggcggtgccagcggcggggcgggcagcggtgcacgtggatccccatccagtaccccatcatcgcggagtgcaagtgctcctgctagacgcgtactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgccgtttccacattcttttctcatccttcttctcctgttttctctgcatcaaggtcagcacgatagcactgtctctctatgcttagggagaggcctgtcctgtacatcccgtgcccccacaagatgcctactacaacaacatcttctgcatgtcctgcatagcagtgttgggagaatgtgcactacttccactcttctgatttctattttatgtgtttgctttataccagtgttgccatttgggaattaatacatggttgatcaaatcaattgcatcacagctgtatcctgtatcagaggaacattatcaaagcttttgttgctgtatttggtatctgacctgcagataaacatgttttaggaaggttttgcaaaagtagctgtgaaatgagctggtgttgtgatttaacctgacaggcagctaaacagtataccacagagctattcacctactttccctcagtgggaaaagggaagagaactgaggggggggggaataaataagtaaataacaaaataaaactcatggattaagaaaaagactttgtactggaatggatgagaagaataatagtaatgataataatatgtcactctgaaagtaatgcctcttatttctgtggagactacaaacatacaaagagcacaacattccatagagcaaattctcagttacagaatgctattttttttttcaacacagtcaaaatcattaatttttttttgcctgcaatggacaagagctttgaagctgttctcgtaaaaatctgtactagcagaagtgacctgcaatcactactgctgaaatgcacaacccaccacatcattgtgctcacattcactgtttggtttctgtaaatgtacaggaattgtctgaaattagatatgattttttttttctccatgaaggaattcaattacacacctttgcctcatgcacttctttgtcatttttgtcagactgcttctctcctgcaatttgtctcatggcaacaaaatataatggagttctgctgggaacttccctactgccataccactatcatctgcctctgacattttggacaaatgtaataaaataggaggtattactttcagagcagaccttgtatgtatttacaaaacaagtggtacacaaaaaaaattgttcatcccaccaaccaatgcccatcctgtccctgaatagtagctgtcccccacagccttgaccagtttaggtcaacagttctgcttctgtcccctcccagctccttgtaacccctcagccccccttgctggcaggacagtatgagaagctgaaaaactagaatgtcctagttctttgcagtgctgctaatcaacaaccaaaacagtggtgtgttaccaatattgttgatatcacagcatcataccattatgaaggaagtaacccagccaaaatcaggtcagcttgctaacaagagaactgtgcataagtttaagatgtgtgtgttcctcagtaccttaaaaaataagtagtaacgttcaaatgagtagaagagtagaactgagcttaaaacatctgtcagacaacagtgaaccaacc

SEQ ID NO: 121 - DNA, 9796 bp, synthetic construct
  misc_feature 1..9796: note = Targeting Vector TV2
  source 1..9796: mol_type = other DNA
gaatgaagggcctgagggtgggcagtctgtctatcatgtacatctccatattctgggaggtcgtcagttgggctggcctcctggctaagatttttgcaccacaagagatgctgcatgtgtacaaatcactagcaaatagatttgtttcccatcaacttagccactgttaatgtaaattgttcttggatatgtgtctttggagggcaataaatgctctgaacagcacttgcacaataaagatacagcatgtgggaatgatctgtctcatgtgtcttactgatggtattggttctgtaagataaaatattgtgtctgggatgtgtttggctctactattaatggtgctctattgattgtgatttgtcatttgaaacctgaggatgcgactgtatagcagtctttcatgcatttttggaaaaaaacttaagctttttgaaagctgctgctacaactttttgtattgttataaagttttgtattgtttttttaattgtgaaattataaagatgccgtgcagggactgtttgaagcaaagtgcattgttttagaaacctacaactctagttcaagcactccatcagtatctgcttaatctttgtcatcctttgctatgagaaaatattaagcagtagtctaaaggtactatgaaactataacatagctgacattgtatttataactacgtcatgattttgatagaattgaggacttgaaaatgttaaactattcatgtagggcctcttaagatgcttaagttgtttagtaatgtaagtgtgcatttaattgagattttattgggcataatttgtccatcagtatgacactccttgtcagtgttgccttatacttgatgttgttaccggatctctgcaaggcagttattcttgaattaggctcattgaagtgtctgccagtataaatatatagcaactgttctttgtgttaaaattgagaagctaaccagtttttagtgcttctgactgttggaattctttaagcagatgccataagaaaattgtatttgtgatcaccacttctccagagtggttttaacaccaagggcattagagaaagaaaggcaggcgtgtagagaatagtggacagacaaaagctgtgagttacgttatgtttttcagctgaaaagctgtgtttggtaaaagcatatgaaatcactcaacttggaagcattctcttagttctctgatagttctgagcagcagaactcttcacctaagaggttacttcaactggaagactacctagtgcttctgatggcaactatatttaagatgagaataagaggtgtttccagtgtggtagcctcacatctgttgcagtggttaccgttcgtcctcctccgagggacacagcttggccattcactgtggtgacaccaatatgatgatcagcaaatggtgtttattcactactaaacacagcttatatacatttttacctacaaaatcgtgctgtcatgtcccactctgattggttcacacatttccccgaaaagtgccacctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttaataacttcgtataatgtatgctatacgaagttataccggtgaagttcctattccgaagttcctattctctagaaagtataggaacttcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcagcggcgccaccaacttcagcctgctgaagcaggccggcgacgtggaggagaaccccggccccgctagctaaagcggccgccaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagaagttcctattccgaagttcctattctctagaaagtataggaacttcgctagcaccggtgccaccatggccacctctgatgaagtcaggaagaacctgatggacatgttcagggacaggcaggccttctctgaacacacctggaagatgctcctgtctgtgtgcagatcctgggctgcctggtgcaagctgaacggtacccatactctttatgcccccggtggatatgacattatgggatatctggaccagatcggcaaccggccaaacccgcaggtggaactgggccccgtggatacatcctgcgccttgattctttgtgacctgaaacagaaagacaccccgatagtttacgcgagtgaagccttcctctacatgacaggttacagcaacgcagaggtgctgggccggaattgccggtttctgcaaagccctgacggcatggtgaagcccaagagcacccggaagtacgtggatagtaacacaatcaatactatgcgcaaggcaatcgacaggaatgccgaggtgcaggttgaagtagtcaattttaaaaagaatggacagcgatttgttaatttcctgactatgatacctgttagggacgaaacaggcgagtatcgatactctatgggattccagtgcgaaacagaaggcggaagcggtggcgtgcccaagaagaagaggaaagtcttcgaaggcagcggcgccaccaacttcagcctgctgaagcaggccggcgacgtggaggagaaccccggccccttcgaagtgcccaagaagaagaggaaagtcggcggacatactctttatgcccccggtggatatgacattatgggatatctgaggcagatcaggaaccggccaaacccgcaggtggaactgggccccgtggatacatcctgcgccttgattctttgtgacctgaaacagaaagacaccccgatagtttacgcgagtgaagccttcctctacatgacaggttacagcaacgcagaggtgctgggccggaattgccggtttctgcaaagccctgacggcatggtgaagcccaagagcacccggaagtacgtggatagtaacacaatcaatactatgcgcaaggcaatcgacaggaatgccgaggtgcaggttgaagtagtcaattttaaaaagaatggacagcgatttgttaatttcctgactatgatacctgttagggacgaaacaggcgagtatcgatactctatgggattccagtgcgaaacagaaggtaccaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaagatggggactaattaattaactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttatacgcgtgccaccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaacgcgtactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgccgtttccacattcttttctcatccttcttctcctgttttctctgcatcaaggtcagcacgatagcactgtctctctatgcttagggagaggcctgtcctgtacatcccgtgcccccacaagatgcctactacaacaacatcttctgcatgtcctgcatagcagtgttgggagaatgtgcactacttccactcttctgatttctattttatgtgtttgctttataccagtgttgccatttgggaattaatacatggttgatcaaatcaattgcatcacagctgtatcctgtatcagaggaacattatcaaagcttttgttgctgtatttggtatctgacctgcagataaacatgttttaggaaggttttgcaaaagtagctgtgaaatgagctggtgttgtgatttaacctgacaggcagctaaacagtataccacagagctattcacctactttccctcagtgggaaaagggaagagaactgaggggggggggaataaataagtaaataacaaaataaaactcatggattaagaaaaagactttgtactggaatggatgagaagaataatagtaatgataataatatgtcactctgaaagtaatgcctcttatttctgtggagactacaaacatacaaagagcacaacattccatagagcaaattctcagttacagaatgctattttttttttcaacacagtcaaaatcattaatttttttttgcctgcaatggacaagagctttgaagctgttctcgtaaaaatctgtactagcagaagtgacctgcaatcactactgctgaaatgcacaacccaccacatcattgtgctcacattcactgtttggtttctgtaaatgtacaggaattgtctgaaattagatatgattttttttttctccatgaaggaattcaattacacacctttgcctcatgcacttctttgtcatttttgtcagactgcttctctcctgcaatttgtctcatggcaacaaaatataatggagttctgctgggaacttccctactgccataccactatcatctgcctctgacattttggacaaatgtaataaaataggaggtattactttcagagcagaccttgtatgtatttacaaaacaagtggtacacaaaaaaaattgttcatcccaccaaccaatgcccatcctgtccctgaatagtagctgtcccccacagccttgaccagtttaggtcaacagttctgcttctgtcccctcccagctccttgtaacccctcagccccccttgctggcaggacagtatgagaagctgaaaaactagaatgtcctagttctttgcagtgctgctaatcaacaaccaaaacagtggtgtgttaccaatattgttgatatcacagcatcataccattatgaaggaagtaacccagccaaaatcaggtcagcttgctaacaagagaactgtgcataagtttaagatgtgtgtgttcctcagtaccttaaaaaataagtagtaacgttcaaatgagtagaagagtagaactgagcttaaaacatctgtcagacaacagtgaaccaacc

SEQ ID NO: 122 - DNA, 9991 bp, synthetic construct
  misc_feature 1..9991: note = Targeting Vector TV3
  source 1..9991: mol_type = other DNA
gaatgaagggcctgagggtgggcagtctgtctatcatgtacatctccatattctgggaggtcgtcagttgggctggcctcctggctaagatttttgcaccacaagagatgctgcatgtgtacaaatcactagcaaatagatttgtttcccatcaacttagccactgttaatgtaaattgttcttggatatgtgtctttggagggcaataaatgctctgaacagcacttgcacaataaagatacagcatgtgggaatgatctgtctcatgtgtcttactgatggtattggttctgtaagataaaatattgtgtctgggatgtgtttggctctactattaatggtgctctattgattgtgatttgtcatttgaaacctgaggatgcgactgtatagcagtctttcatgcatttttggaaaaaaacttaagctttttgaaagctgctgctacaactttttgtattgttataaagttttgtattgtttttttaattgtgaaattataaagatgccgtgcagggactgtttgaagcaaagtgcattgttttagaaacctacaactctagttcaagcactccatcagtatctgcttaatctttgtcatcctttgctatgagaaaatattaagcagtagtctaaaggtactatgaaactataacatagctgacattgtatttataactacgtcatgattttgatagaattgaggacttgaaaatgttaaactattcatgtagggcctcttaagatgcttaagttgtttagtaatgtaagtgtgcatttaattgagattttattgggcataatttgtccatcagtatgacactccttgtcagtgttgccttatacttgatgttgttaccggatctctgcaaggcagttattcttgaattaggctcattgaagtgtctgccagtataaatatatagcaactgttctttgtgttaaaattgagaagctaaccagtttttagtgcttctgactgttggaattctttaagcagatgccataagaaaattgtatttgtgatcaccacttctccagagtggttttaacaccaagggcattagagaaagaaaggcaggcgtgtagagaatagtggacagacaaaagctgtgagttacgttatgtttttcagctgaaaagctgtgtttggtaaaagcatatgaaatcactcaacttggaagcattctcttagttctctgatagttctgagcagcagaactcttcacctaagaggttacttcaactggaagactacctagtgcttctgatggcaactatatttaagatgagaataagaggtgtttccagtgtggtagcctcacatctgttgcagtggttaccgttcgtcctcctccgagggacacagcttggccattcactgtggtgacaccaatatgatgatcagcaaatggtgtttattcactactaaacacagcttatatacatttttacctacaaaatcgtgctgtcatgtcccactctgattggttcacacatttccccgaaaagtgccacctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttaataacttcgtataatgtatgctatacgaagttataccggtgaagttcctattccgaagttcctattctctagaaagtataggaacttcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcagcggcgccaccaacttcagcctgctgaagcaggccggcgacgtggaggagaaccccggccccgctagctaaagcggccgccaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagaagttcctattccgaagttcctattctctagaaagtataggaacttcgctagcaccggtgccaccatggccacctctgatgaagtcaggaagaacctgatggacatgttcagggacaggcaggccttctctgaacacacctggaagatgctcctgtctgtgtgcagatcctgggctgcctggtgcaagctgaacggtacccatactctttatgcccccggtggatatgacattatgggatatctggaccagatcggcaaccggccaaacccgcaggtggaactgggccccgtggatacatcctgcgccttgattctttgtgacctgaaacagaaagacaccccgatagtttacgcgagtgaagccttcctctacatgacaggttacagcaacgcagaggtgctgggccggaattgccggtttctgcaaagccctgacggcatggtgaagcccaagagcacccggaagtacgtggatagtaacacaatcaatactatgcgcaaggcaatcgacaggaatgccgaggtgcaggttgaagtagtcaattttaaaaagaatggacagcgatttgttaatttcctgactatgatacctgttagggacgaaacaggcgagtatcgatactctatgggattccagtgcgaaacagaaggcggaagcggtggcgtgcccaagaagaagaggaaagtcttcgaaggcagcggcgccaccaacttcagcctgctgaagcaggccggcgacgtggaggagaaccccggccccttcgaagtgcccaagaagaagaggaaagtcggcggacatactctttatgcccccggtggatatgacattatgggatatctgaggcagatcaggaaccggccaaacccgcaggtggaactgggccccgtggatacatcctgcgccttgattctttgtgacctgaaacagaaagacaccccgatagtttacgcgagtgaagccttcctctacatgacaggttacagcaacgcagaggtgctgggccggaattgccggtttctgcaaagccctgacggcatggtgaagcccaagagcacccggaagtacgtggatagtaacacaatcaatactatgcgcaaggcaatcgacaggaatgccgaggtgcaggttgaagtagtcaattttaaaaagaatggacagcgatttgttaatttcctgactatgatacctgttagggacgaaacaggcgagtatcgatactctatgggattccagtgcgaaacagaaggtaccaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaagatggggactaattaattaactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttatacgcgtgccaccatgatgacagacataaaagatggaccacgctcaggggaagatgtatcagctgcaagatctttccctggttccaaaggaatgaacttacctgctagcaagtctgtggcctctggaattctgcctgatgacagttacagaatggattatccagagataggagtatgtgttataataaacaataagaacttccaccgagataccggactgtcatctcgttcaggcacggatgcagatgctgcaagtgtcagagaagtttttatgaagctgggatataaagtcaagcttaacaatgatctgtcaagcagagatatttttaagctattgaaaaatgtttctgaagaagatcacagcaagcgaagcagttttgtttgtgtgttgctaagccatggcgatgaaggactcttctatggtacagatggccctcttgaactgaaagtactaaccagccttttcagaggtgacaagtgcagaagtctagcagggaaacccaaactctttttcattcaggcctgtagaggaacagaattagattctggtattgaagcagccagtggaccagatgaaacagtgtgtcaaaaaatacctgtagaagcagacttcctgtatgcatattctactgctccaggctactactcctggaggaacgcagctgaaggctcctggtttattcagtctctgtgtaggatgctgaaggaacacgccaggaaacttgaactcatgcagattttaactcgtgtaaatcgcagagtggcagaatatgaatcctgctccactcgacaggatttcaatgcaaagaaacagattccatgcattgagtctatgcttaccaaagaattctactttccttgctaaacgcgtactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgccgtttccacattcttttctcatccttcttctcctgttttctctgcatcaaggtcagcacgatagcactgtctctctatgcttagggagaggcctgtcctgtacatcccgtgcccccacaagatgcctactacaacaacatcttctgcatgtcctgcatagcagtgttgggagaatgtgcactacttccactcttctgatttctattttatgtgtttgctttataccagtgttgccatttgggaattaatacatggttgatcaaatcaattgcatcacagctgtatcctgtatcagaggaacattatcaaagcttttgttgctgtatttggtatctgacctgcagataaacatgttttaggaaggttttgcaaaagtagctgtgaaatgagctggtgttgtgatttaacctgacaggcagctaaacagtataccacagagctattcacctactttccctcagtgggaaaagggaagagaactgaggggggggggaataaataagtaaataacaaaataaaactcatggattaagaaaaagactttgtactggaatggatgagaagaataatagtaatgataataatatgtcactctgaaagtaatgcctcttatttctgtggagactacaaacatacaaagagcacaacattccatagagcaaattctcagttacagaatgctattttttttttcaacacagtcaaaatcattaatttttttttgcctgcaatggacaagagctttgaagctgttctcgtaaaaatctgtactagcagaagtgacctgcaatcactactgctgaaatgcacaacccaccacatcattgtgctcacattcactgtttggtttctgtaaatgtacaggaattgtctgaaattagatatgattttttttttctccatgaaggaattcaattacacacctttgcctcatgcacttctttgtcatttttgtcagactgcttctctcctgcaatttgtctcatggcaacaaaatataatggagttctgctgggaacttccctactgccataccactatcatctgcctctgacattttggacaaatgtaataaaataggaggtattactttcagagcagaccttgtatgtatttacaaaacaagtggtacacaaaaaaaattgttcatcccaccaaccaatgcccatcctgtccctgaatagtagctgtcccccacagccttgaccagtttaggtcaacagttctgcttctgtcccctcccagctccttgtaacccctcagccccccttgctggcaggacagtatgagaagctgaaaaactagaatgtcctagttctttgcagtgctgctaatcaacaaccaaaacagtggtgtgttaccaatattgttgatatcacagcatcataccattatgaaggaagtaacccagccaaaatcaggtcagcttgctaacaagagaactgtgcataagtttaagatgtgtgtgttcctcagtaccttaaaaaataagtagtaacgttcaaatgagtagaagagtagaactgagcttaaaacatctgtcagacaacagtgaaccaacc

SEQ ID NO: 123 - DNA, 9859 bp, synthetic construct
  misc_feature 1..9859: note = Targeting Vector TV4
  source 1..9859: mol_type = other DNA
gaatgaagggcctgagggtgggcagtctgtctatcatgtacatctccatattctgggaggtcgtcagttgggctggcctcctggctaagatttttgcaccacaagagatgctgcatgtgtacaaatcactagcaaatagatttgtttcccatcaacttagccactgttaatgtaaattgttcttggatatgtgtctttggagggcaataaatgctctgaacagcacttgcacaataaagatacagcatgtgggaatgatctgtctcatgtgtcttactgatggtattggttctgtaagataaaatattgtgtctgggatgtgtttggctctactattaatggtgctctattgattgtgatttgtcatttgaaacctgaggatgcgactgtatagcagtctttcatgcatttttggaaaaaaacttaagctttttgaaagctgctgctacaactttttgtattgttataaagttttgtattgtttttttaattgtgaaattataaagatgccgtgcagggactgtttgaagcaaagtgcattgttttagaaacctacaactctagttcaagcactccatcagtatctgcttaatctttgtcatcctttgctatgagaaaatattaagcagtagtctaaaggtactatgaaactataacatagctgacattgtatttataactacgtcatgattttgatagaattgaggacttgaaaatgttaaactattcatgtagggcctcttaagatgcttaagttgtttagtaatgtaagtgtgcatttaattgagattttattgggcataatttgtccatcagtatgacactccttgtcagtgttgccttatacttgatgttgttaccggatctctgcaaggcagttattcttgaattaggctcattgaagtgtctgccagtataaatatatagcaactgttctttgtgttaaaattgagaagctaaccagtttttagtgcttctgactgttggaattctttaagcagatgccataagaaaattgtatttgtgatcaccacttctccagagtggttttaacaccaagggcattagagaaagaaaggcaggcgtgtagagaatagtggacagacaaaagctgtgagttacgttatgtttttcagctgaaaagctgtgtttggtaaaagcatatgaaatcactcaacttggaagcattctcttagttctctgatagttctgagcagcagaactcttcacctaagaggttacttcaactggaagactacctagtgcttctgatggcaactatatttaagatgagaataagaggtgtttccagtgtggtagcctcacatctgttgcagtggttaccgttcgtcctcctccgagggacacagcttggccattcactgtggtgacaccaatatgatgatcagcaaatggtgtttattcactactaaacacagcttatatacatttttacctacaaaatcgtgctgtcatgtcccactctgattggttcacacatttccccgaaaagtgccacctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttaataacttcgtataatgtatgctatacgaagttataccggtgaagttcctattccgaagttcctattctctagaaagtataggaacttcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcagcggcgccaccaacttcagcctgctgaagcaggccggcgacgtggaggagaaccccggccccgctagctaaagcggccgccaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagaagttcctattccgaagttcctattctctagaaagtataggaacttcgctagcaccggtgccaccatggccacctctgatgaagtcaggaagaacctgatggacatgttcagggacaggcaggccttctctgaacacacctggaagatgctcctgtctgtgtgcagatcctgggctgcctggtgcaagctgaacggtacccatactctttatgcccccggtggatatgacattatgggatatctggaccagatcggcaaccggccaaacccgcaggtggaactgggccccgtggatacatcctgcgccttgattctttgtgacctgaaacagaaagacaccccgatagtttacgcgagtgaagccttcctctacatgacaggttacagcaacgcagaggtgctgggccggaattgccggtttctgcaaagccctgacggcatggtgaagcccaagagcacccggaagtacgtggatagtaacacaatcaatactatgcgcaaggcaatcgacaggaatgccgaggtgcaggttgaagtagtcaattttaaaaagaatggacagcgatttgttaatttcctgactatgatacctgttagggacgaaacaggcgagtatcgatactctatgggattccagtgcgaaacagaaggcggaagcggtggcgtgcccaagaagaagaggaaagtcttcgaaggcagcggcgccaccaacttcagcctgctgaagcaggccggcgacgtggaggagaaccccggccccttcgaagtgcccaagaagaagaggaaagtcggcggacatactctttatgcccccggtggatatgacattatgggatatctgaggcagatcaggaaccggccaaacccgcaggtggaactgggccccgtggatacatcctgcgccttgattctttgtgacctgaaacagaaagacaccccgatagtttacgcgagtgaagccttcctctacatgacaggttacagcaacgcagaggtgctgggccggaattgccggtttctgcaaagccctgacggcatggtgaagcccaagagcacccggaagtacgtggatagtaacacaatcaatactatgcgcaaggcaatcgacaggaatgccgaggtgcaggttgaagtagtcaattttaaaaagaatggacagcgatttgttaatttcctgactatgatacctgttagggacgaaacaggcgagtatcgatactctatgggattccagtgcgaaacagaaggtaccaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaagatggggactaattaattaactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttatacgcgtgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgtggcggctagacgcgtactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgccgtttccacattcttttctcatccttcttctcctgttttctctgcatcaaggtcagcacgatagcactgtctctctatgcttagggagaggcctgtcctgtacatcccgtgcccccacaagatgcctactacaacaacatcttctgcatgtcctgcatagcagtgttgggagaatgtgcactacttccactcttctgatttctattttatgtgtttgctttataccagtgttgccatttgggaattaatacatggttgatcaaatcaattgcatcacagctgtatcctgtatcagaggaacattatcaaagcttttgttgctgtatttggtatctgacctgcagataaacatgttttaggaaggttttgcaaaagtagctgtgaaatgagctggtgttgtgatttaacctgacaggcagctaaacagtataccacagagctattcacctactttccctcagtgggaaaagggaagagaactgaggggggggggaataaataagtaaataacaaaataaaactcatggattaagaaaaagactttgtactggaatggatgagaagaataatagtaatgataataatatgtcactctgaaagtaatgcctcttatttctgtggagactacaaacatacaaagagcacaacattccatagagcaaattctcagttacagaatgctattttttttttcaacacagtcaaaatcattaatttttttttgcctgcaatggacaagagctttgaagctgttctcgtaaaaatctgtactagcagaagtgacctgcaatcactactgctgaaatgcacaacccaccacatcattgtgctcacattcactgtttggtttctgtaaatgtacaggaattgtctgaaattagatatgattttttttttctccatgaaggaattcaattacacacctttgcctcatgcacttctttgtcatttttgtcagactgcttctctcctgcaatttgtctcatggcaacaaaatataatggagttctgctgggaacttccctactgccataccactatcatctgcctctgacattttggacaaatgtaataaaataggaggtattactttcagagcagaccttgtatgtatttacaaaacaagtggtacacaaaaaaaattgttcatcccaccaaccaatgcccatcctgtccctgaatagtagctgtcccccacagccttgaccagtttaggtcaacagttctgcttctgtcccctcccagctccttgtaacccctcagccccccttgctggcaggacagtatgagaagctgaaaaactagaatgtcctagttctttgcagtgctgctaatcaacaaccaaaacagtggtgtgttaccaatattgttgatatcacagcatcataccattatgaaggaagtaacccagccaaaatcaggtcagcttgctaacaagagaactgtgcataagtttaagatgtgtgtgttcctcagtaccttaaaaaataagtagtaacgttcaaatgagtagaagagtagaactgagcttaaaacatctgtcagacaacagtgaaccaacc

SEQ ID NO: 124 - DNA, 11581 bp, synthetic construct
  misc_feature 1..11581: note = Targeting Vector TV5
  source 1..11581: mol_type = other DNA
gaatgaagggcctgagggtgggcagtctgtctatcatgtacatctccatattctgggaggtcgtcagttgggctggcctcctggctaagatttttgcaccacaagagatgctgcatgtgtacaaatcactagcaaatagatttgtttcccatcaacttagccactgttaatgtaaattgttcttggatatgtgtctttggagggcaataaatgctctgaacagcacttgcacaataaagatacagcatgtgggaatgatctgtctcatgtgtcttactgatggtattggttctgtaagataaaatattgtgtctgggatgtgtttggctctactattaatggtgctctattgattgtgatttgtcatttgaaacctgaggatgcgactgtatagcagtctttcatgcatttttggaaaaaaacttaagctttttgaaagctgctgctacaactttttgtattgttataaagttttgtattgtttttttaattgtgaaattataaagatgccgtgcagggactgtttgaagcaaagtgcattgttttagaaacctacaactctagttcaagcactccatcagtatctgcttaatctttgtcatcctttgctatgagaaaatattaagcagtagtctaaaggtactatgaaactataacatagctgacattgtatttataactacgtcatgattttgatagaattgaggacttgaaaatgttaaactattcatgtagggcctcttaagatgcttaagttgtttagtaatgtaagtgtgcatttaattgagattttattgggcataatttgtccatcagtatgacactccttgtcagtgttgccttatacttgatgttgttaccggatctctgcaaggcagttattcttgaattaggctcattgaagtgtctgccagtataaatatatagcaactgttctttgtgttaaaattgagaagctaaccagtttttagtgcttctgactgttggaattctttaagcagatgccataagaaaattgtatttgtgatcaccacttctccagagtggttttaacaccaagggcattagagaaagaaaggcaggcgtgtagagaatagtggacagacaaaagctgtgagttacgttatgtttttcagctgaaaagctgtgtttggtaaaagcatatgaaatcactcaacttggaagcattctcttagttctctgatagttctgagcagcagaactcttcacctaagaggttacttcaactggaagactacctagtgcttctgatggcaactatatttaagatgagaataagaggtgtttccagtgtggtagcctcacatctgttgcagtggttaccgttcgtcctcctccgagggacacagcttggccattcactgtggtgacaccaatatgatgatcagcaaatggtgtttattcactactaaacacagcttatatacatttttacctacaaaatcgtgctgtcatgtcccactctgattggttcacacatttccccgaaaagtgccacctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttaataacttcgtataatgtatgctatacgaagttataccggtgaagttcctattccgaagttcctattctctagaaagtataggaacttcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcagcggcgccaccaacttcagcctgctgaagcaggccggcgacgtggaggagaaccccggccccgctagctaaagcggccgccaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagaagttcctattccgaagttcctattctctagaaagtataggaacttcgctagcaccggtgccaccatgaatggagctataggaggtgaccttttgctcaattttcctgacatgtcggtcctagagcgccaaagggctcacctcaagtacctcaatcccacctttgattctcctctcgccggcttctttgccgattcttcaatgattaccggcggcgagatggacagctatctttcgactgccggtttgaatcttccgatgatgtacggtgagacgacggtggaaggtgattcaagactctcaatttcgccggaaacgacgcttgggactggaaatttcaagaaacggaagtttgatacagagactaaggattgtaatgagaagaagaagaagatgacgatgaacagagatgacctagtagaagaaggagaagaagagaagtcgaaaataacagagcaaaacaatgggagcacaaaaagcatcaagaagatgaaacacaaagccaagaaagaagagaacaatttctctaatgattcatctaaagtgacgaaggaattggagaaaacggattatattcatggtggcggtggctctggaggtggtgggtccggaggaggcggccgccgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagcccgggtaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgcctaaaaagaagcgtaaagtcatgaagatgaagatggacaaaaagactatagtttggtttagaagagacctaaggattgaggataatcctgcattagcagcagctgctcacgaaggatctgtttttcctgtcttcatttggtgtcctgaagaagaaggacagttttatcctggaagagcttcaagatggtggatgaaacaatcacttgctcacttatctcaatccttgaaggctcttggatctgacctcactttaatcaaaacccacaacacgatttcagcgatcttggattgtatccgcgttaccggtgctacaaaagtcgtctttaaccacctctatgatcctgtttcgttagttcgggaccataccgtaaaggagaagctggtggaacgtgggatctctgtgcaaagctacaatggagatctattgtatgaaccgtgggagatatactgcgaaaagggcaaaccttttacgagtttcaattcttactggaagaaatgcttagatatgtcgattgaatccgttatgcttcctcctccttggcggttgatgccaataactgcagcggctgaagcgatttgggcgtgttcgattgaagaactagggctggagaatgaggccgagaaaccgagcaatgcgttgttaactagagcttggtctccaggatggagcaatgctgataagttactaaatgagttcatcgagaagcagttgatagattatgcaaagaacagcaagaaagttgttgggaattctacttcactactttctccgtatctccatttcggggaaataagcgtcagacacgttttccagtgtgcccggatgaaacaaattatatgggcaagagataagaacagtgaaggagaagaaagtgcagatctttttcttaggggaatcggtttaagagagtattctcggtatatatgtttcaacttcccgtttactcacgagcaatcgttgttgagtcatcttcggtttttcccttgggatgctgatgttgataagttcaaggcctggagacaaggcaggaccggttatccgttggtggatgccggaatgagagagttttgggctaccggatggatgcataacagaataagagtgattgtttcaagctttgctgtgaagtttcttctccttccatggaaatggggaatgaagtatttctgggatacacttttggatgctgatttggaatgtgacatccttggctggcagtatatctctgggagtatccccgatggccacgagcttgatcgcttggacaatcccgcgttacaaggcgccaaatatgacccagaaggtgagtacataaggcaatggcttcccgagcttgcgagattgccaactgaatggatccatcatccatgggacgctcctttaaccgtactcaaagcttctggtgtggaactcggaacaaactatgcgaaacccattgtagacatcgacacagctcgtgagctactagctaaagctatttcaagaacccgtgaagcacagatcatgatcggagcagcacctgatgagattgtagcagatagcttcgaggccttaggggctaataccattaaagaacctggtctttgcccatctgtgtcttctaatgaccaacaagtaccttcggctgttggtggcggtggctctggaggtggtgggtccggaggaggcggccgcacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgtaattaattaactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttatacgcgtgccaccatggatcattcccagtgccttgtgactatatacgccgcggcggtgctgctggggctccggctgcagcagggctcctgccagcactacctgcacatccgcccggctcccagcgacaacctgcccctggtggatctaatcgagcacccggaccctatctttgaccccaaggagaaggatcttaacgagaccttgctaaggagcctcatgggaggacacttcgaccctaactttatggctatgtccctgcccgaggaccggctcggggtagacgatctggccgagctggacttgctgctgcggcagagaccctcgggagcgatgcccggcgaaatcaaggggctggagttctacgacgggctgcagccgggcaagaagcacaggctgagcaagaagctgcgcaggaagctgcagatgtggctctggtcccagaccttctgcccggtcctatacacgtggaacgatctcggcagccgcttttggccccggtacgtcaaagtgggcagctgctacagtaaaaggtcttgctctgtcccagaaggcatggtctgcaaacctgccaagtccgtgcatttaacgatcctgaggtggcggtgccagcggcggggcgggcagcggtgcacgtggatccccatccagtaccccatcatcgcggagtgcaagtgctcctgctagacgcgtactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgccgtttccacattcttttctcatccttcttctcctgttttctctgcatcaaggtcagcacgatagcactgtctctctatgcttagggagaggcctgtcctgtacatcccgtgcccccacaagatgcctactacaacaacatcttctgcatgtcctgcatagcagtgttgggagaatgtgcactacttccactcttctgatttctattttatgtgtttgctttataccagtgttgccatttgggaattaatacatggttgatcaaatcaattgcatcacagctgtatcctgtatcagaggaacattatcaaagcttttgttgctgtatttggtatctgacctgcagataaacatgttttaggaaggttttgcaaaagtagctgtgaaatgagctggtgttgtgatttaacctgacaggcagctaaacagtataccacagagctattcacctactttccctcagtgggaaaagggaagagaactgaggggggggggaataaataagtaaataacaaaataaaactcatggattaagaaaaagactttgtactggaatggatgagaagaataatagtaatgataataatatgtcactctgaaagtaatgcctcttatttctgtggagactacaaacatacaaagagcacaacattccatagagcaaattctcagttacagaatgctattttttttttcaacacagtcaaaatcattaatttttttttgcctgcaatggacaagagctttgaagctgttctcgtaaaaatctgtactagcagaagtgacctgcaatcactactgctgaaatgcacaacccaccacatcattgtgctcacattcactgtttggtttctgtaaatgtacaggaattgtctgaaattagatatgattttttttttctccatgaaggaattcaattacacacctttgcctcatgcacttctttgtcatttttgtcagactgcttctctcctgcaatttgtctcatggcaacaaaatataatggagttctgctgggaacttccctactgccataccactatcatctgcctctgacattttggacaaatgtaataaaataggaggtattactttcagagcagaccttgtatgtatttacaaaacaagtggtacacaaaaaaaattgttcatcccaccaaccaatgcccatcctgtccctgaatagtagctgtcccccacagccttgaccagtttaggtcaacagttctgcttctgtcccctcccagctccttgtaacccctcagccccccttgctggcaggacagtatgagaagctgaaaaactagaatgtcctagttctttgcagtgctgctaatcaacaaccaaaacagtggtgtgttaccaatattgttgatatcacagcatcataccattatgaaggaagtaacccagccaaaatcaggtcagcttgctaacaagagaactgtgcataagtttaagatgtgtgtgttcctcagtaccttaaaaaataagtagtaacgttcaaatgagtagaagagtagaactgagcttaaaacatctgtcagacaacagtgaaccaacc

SEQ ID NO: 125 - DNA, 11566 bp, synthetic construct
  misc_feature 1..11566: note = Targeting Vector TV6
  source 1..11566: mol_type = other DNA
gaatgaagggcctgagggtgggcagtctgtctatcatgtacatctccatattctgggaggtcgtcagttgggctggcctcctggctaagatttttgcaccacaagagatgctgcatgtgtacaaatcactagcaaatagatttgtttcccatcaacttagccactgttaatgtaaattgttcttggatatgtgtctttggagggcaataaatgctctgaacagcacttgcacaataaagatacagcatgtgggaatgatctgtctcatgtgtcttactgatggtattggttctgtaagataaaatattgtgtctgggatgtgtttggctctactattaatggtgctctattgattgtgatttgtcatttgaaacctgaggatgcgactgtatagcagtctttcatgcatttttggaaaaaaacttaagctttttgaaagctgctgctacaactttttgtattgttataaagttttgtattgtttttttaattgtgaaattataaagatgccgtgcagggactgtttgaagcaaagtgcattgttttagaaacctacaactctagttcaagcactccatcagtatctgcttaatctttgtcatcctttgctatgagaaaatattaagcagtagtctaaaggtactatgaaactataacatagctgacattgtatttataactacgtcatgattttgatagaattgaggacttgaaaatgttaaactattcatgtagggcctcttaagatgcttaagttgtttagtaatgtaagtgtgcatttaattgagattttattgggcataatttgtccatcagtatgacactccttgtcagtgttgccttatacttgatgttgttaccggatctctgcaaggcagttattcttgaattaggctcattgaagtgtctgccagtataaatatatagcaactgttctttgtgttaaaattgagaagctaaccagtttttagtgcttctgactgttggaattctttaagcagatgccataagaaaattgtatttgtgatcaccacttctccagagtggttttaacaccaagggcattagagaaagaaaggcaggcgtgtagagaatagtggacagacaaaagctgtgagttacgttatgtttttcagctgaaaagctgtgtttggtaaaagcatatgaaatcactcaacttggaagcattctcttagttctctgatagttctgagcagcagaactcttcacctaagaggttacttcaactggaagactacctagtgcttctgatggcaactatatttaagatgagaataagaggtgtttccagtgtggtagcctcacatctgttgcagtggttaccgttcgtcctcctccgagggacacagcttggccattcactgtggtgacaccaatatgatgatcagcaaatggtgtttattcactactaaacacagcttatatacatttttacctacaaaatcgtgctgtcatgtcccactctgattggttcacacatttccccgaaaagtgccacctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttaataacttcgtataatgtatgctatacgaagttataccggtgaagttcctattccgaagttcctattctctagaaagtataggaacttcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcagcggcgccaccaacttcagcctgctgaagcaggccggcgacgtggaggagaaccccggccccgctagctaaagcggccgccaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagaagttcctattccgaagttcctattctctagaaagtataggaacttcgctagcaccggtgccaccatgaatggagctataggaggtgaccttttgctcaattttcctgacatgtcggtcctagagcgccaaagggctcacctcaagtacctcaatcccacctttgattctcctctcgccggcttctttgccgattcttcaatgattaccggcggcgagatggacagctatctttcgactgccggtttgaatcttccgatgatgtacggtgagacgacggtggaaggtgattcaagactctcaatttcgccggaaacgacgcttgggactggaaatttcaagaaacggaagtttgatacagagactaaggattgtaatgagaagaagaagaagatgacgatgaacagagatgacctagtagaagaaggagaagaagagaagtcgaaaataacagagcaaaacaatgggagcacaaaaagcatcaagaagatgaaacacaaagccaagaaagaagagaacaatttctctaatgattcatctaaagtgacgaaggaattggagaaaacggattatattcatggtggcggtggctctggaggtggtgggtccggaggaggcggccgccgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagcccgggtaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgcctaaaaagaagcgtaaagtcatgaagatgaagatggacaaaaagactatagtttggtttagaagagacctaaggattgaggataatcctgcattagcagcagctgctcacgaaggatctgtttttcctgtcttcatttggtgtcctgaagaagaaggacagttttatcctggaagagcttcaagatggtggatgaaacaatcacttgctcacttatctcaatccttgaaggctcttggatctgacctcactttaatcaaaacccacaacacgatttcagcgatcttggattgtatccgcgttaccggtgctacaaaagtcgtctttaaccacctctatgatcctgtttcgttagttcgggaccataccgtaaaggagaagctggtggaacgtgggatctctgtgcaaagctacaatggagatctattgtatgaaccgtgggagatatactgcgaaaagggcaaaccttttacgagtttcaattcttactggaagaaatgcttagatatgtcgattgaatccgttatgcttcctcctccttggcggttgatgccaataactgcagcggctgaagcgatttgggcgtgttcgattgaagaactagggctggagaatgaggccgagaaaccgagcaatgcgttgttaactagagcttggtctccaggatggagcaatgctgataagttactaaatgagttcatcgagaagcagttgatagattatgcaaagaacagcaagaaagttgttgggaattctacttcactactttctccgtatctccatttcggggaaataagcgtcagacacgttttccagtgtgcccggatgaaacaaattatatgggcaagagataagaacagtgaaggagaagaaagtgcagatctttttcttaggggaatcggtttaagagagtattctcggtatatatgtttcaacttcccgtttactcacgagcaatcgttgttgagtcatcttcggtttttcccttgggatgctgatgttgataagttcaaggcctggagacaaggcaggaccggttatccgttggtggatgccggaatgagagagttttgggctaccggatggatgcataacagaataagagtgattgtttcaagctttgctgtgaagtttcttctccttccatggaaatggggaatgaagtatttctgggatacacttttggatgctgatttggaatgtgacatccttggctggcagtatatctctgggagtatccccgatggccacgagcttgatcgcttggacaatcccgcgttacaaggcgccaaatatgacccagaaggtgagtacataaggcaatggcttcccgagcttgcgagattgccaactgaatggatccatcatccatgggacgctcctttaaccgtactcaaagcttctggtgtggaactcggaacaaactatgcgaaacccattgtagacatcgacacagctcgtgagctactagctaaagctatttcaagaacccgtgaagcacagatcatgatcggagcagcacctgatgagattgtagcagatagcttcgaggccttaggggctaataccattaaagaacctggtctttgcccatctgtgtcttctaatgaccaacaagtaccttcggctgttggtggcggtggctctggaggtggtgggtccggaggaggcggccgcacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgtaattaattaactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttatacgcgtgccaccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaacgcgtactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgccgtttccacattcttttctcatccttcttctcctgttttctctgcatcaaggtcagcacgatagcactgtctctctatgcttagggagaggcctgtcctgtacatcccgtgcccccacaagatgcctactacaacaacatcttctgcatgtcctgcatagcagtgttgggagaatgtgcactacttccactcttctgatttctattttatgtgtttgctttataccagtgttgccatttgggaattaatacatggttgatcaaatcaattgcatcacagctgtatcctgtatcagaggaacattatcaaagcttttgttgctgtatttggtatctgacctgcagataaacatgttttaggaaggttttgcaaaagtagctgtgaaatgagctggtgttgtgatttaacctgacaggcagctaaacagtataccacagagctattcacctactttccctcagtgggaaaagggaagagaactgaggggggggggaataaataagtaaataacaaaataaaactcatggattaagaaaaagactttgtactggaatggatgagaagaataatagtaatgataataatatgtcactctgaaagtaatgcctcttatttctgtggagactacaaacatacaaagagcacaacattccatagagcaaattctcagttacagaatgctattttttttttcaacacagtcaaaatcattaatttttttttgcctgcaatggacaagagctttgaagctgttctcgtaaaaatctgtactagcagaagtgacctgcaatcactactgctgaaatgcacaacccaccacatcattgtgctcacattcactgtttggtttctgtaaatgtacaggaattgtctgaaattagatatgattttttttttctccatgaaggaattcaattacacacctttgcctcatgcacttctttgtcatttttgtcagactgcttctctcctgcaatttgtctcatggcaacaaaatataatggagttctgctgggaacttccctactgccataccactatcatctgcctctgacattttggacaaatgtaataaaataggaggtattactttcagagcagaccttgtatgtatttacaaaacaagtggtacacaaaaaaaattgttcatcccaccaaccaatgcccatcctgtccctgaatagtagctgtcccccacagccttgaccagtttaggtcaacagttctgcttctgtcccctcccagctccttgtaacccctcagccccccttgctggcaggacagtatgagaagctgaaaaactagaatgtcctagttctttgcagtgctgctaatcaacaaccaaaacagtggtgtgttaccaatattgttgatatcacagcatcataccattatgaaggaagtaacccagccaaaatcaggtcagcttgctaacaagagaactgtgcataagtttaagatgtgtgtgttcctcagtaccttaaaaaataagtagtaacgttcaaatgagtagaagagtagaactgagcttaaaacatctgtcagacaacagtgaaccaacc

SEQ ID NO: 126 - DNA, 11761 bp, synthetic construct
  misc_feature 1..11761: note = Targeting Vector TV7
  source 1..11761: mol_type = other DNA
gaatgaagggcctgagggtgggcagtctgtctatcatgtacatctccatattctgggaggtcgtcagttgggctggcctcctggctaagatttttgcaccacaagagatgctgcatgtgtacaaatcactagcaaatagatttgtttcccatcaacttagccactgttaatgtaaattgttcttggatatgtgtctttggagggcaataaatgctctgaacagcacttgcacaataaagatacagcatgtgggaatgatctgtctcatgtgtcttactgatggtattggttctgtaagataaaatattgtgtctgggatgtgtttggctctactattaatggtgctctattgattgtgatttgtcatttgaaacctgaggatgcgactgtatagcagtctttcatgcatttttggaaaaaaacttaagctttttgaaagctgctgctacaactttttgtattgttataaagttttgtattgtttttttaattgtgaaattataaagatgccgtgcagggactgtttgaagcaaagtgcattgttttagaaacctacaactctagttcaagcactccatcagtatctgcttaatctttgtcatcctttgctatgagaaaatattaagcagtagtctaaaggtactatgaaactataacatagctgacattgtatttataactacgtcatgattttgatagaattgaggacttgaaaatgttaaactattcatgtagggcctcttaagatgcttaagttgtttagtaatgtaagtgtgcatttaattgagattttattgggcataatttgtccatcagtatgacactccttgtcagtgttgccttatacttgatgttgttaccggatctctgcaaggcagttattcttgaattaggctcattgaagtgtctgccagtataaatatatagcaactgttctttgtgttaaaattgagaagctaaccagtttttagtgcttctgactgttggaattctttaagcagatgccataagaaaattgtatttgtgatcaccacttctccagagtggttttaacaccaagggcattagagaaagaaaggcaggcgtgtagagaatagtggacagacaaaagctgtgagttacgttatgtttttcagctgaaaagctgtgtttggtaaaagcatatgaaatcactcaacttggaagcattctcttagttctctgatagttctgagcagcagaactcttcacctaagaggttacttcaactggaagactacctagtgcttctgatggcaactatatttaagatgagaataagaggtgtttccagtgtggtagcctcacatctgttgcagtggttaccgttcgtcctcctccgagggacacagcttggccattcactgtggtgacaccaatatgatgatcagcaaatggtgtttattcactactaaacacagcttatatacatttttacctacaaaatcgtgctgtcatgtcccactctgattggttcacacatttccccgaaaagtgccacctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttaataacttcgtataatgtatgctatacgaagttataccggtgaagttcctattccgaagttcctattctctagaaagtataggaacttcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcagcggcgccaccaacttcagcctgctgaagcaggccggcgacgtggaggagaaccccggccccgctagctaaagcggccgccaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagaagttcctattccgaagttcctattctctagaaagtataggaacttcgctagcaccggtgccaccatgaatggagctataggaggtgaccttttgctcaattttcctgacatgtcggtcctagagcgccaaagggctcacctcaagtacctcaatcccacctttgattctcctctcgccggcttctttgccgattcttcaatgattaccggcggcgagatggacagctatctttcgactgccggtttgaatcttccgatgatgtacggtgagacgacggtggaaggtgattcaagactctcaatttcgccggaaacgacgcttgggactggaaatttcaagaaacggaagtttgatacagagactaaggattgtaatgagaagaagaagaagatgacgatgaacagagatgacctagtagaagaaggagaagaagagaagtcgaaaataacagagcaaaacaatgggagcacaaaaagcatcaagaagatgaaacacaaagccaagaaagaagagaacaatttctctaatgattcatctaaagtgacgaaggaattggagaaaacggattatattcatggtggcggtggctctggaggtggtgggtccggaggaggcggccgccgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagcccgggtaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgcctaaaaagaagcgtaaagtcatgaagatgaagatggacaaaaagactatagtttggtttagaagagacctaaggattgaggataatcctgcattagcagcagctgctcacgaaggatctgtttttcctgtcttcatttggtgtcctgaagaagaaggacagttttatcctggaagagcttcaagatggtggatgaaacaatcacttgctcacttatctcaatccttgaaggctcttggatctgacctcactttaatcaaaacccacaacacgatttcagcgatcttggattgtatccgcgttaccggtgctacaaaagtcgtctttaaccacctctatgatcctgtttcgttagttcgggaccataccgtaaaggagaagctggtggaacgtgggatctctgtgcaaagctacaatggagatctattgtatgaaccgtgggagatatactgcgaaaagggcaaaccttttacgagtttcaattcttactggaagaaatgcttagatatgtcgattgaatccgttatgcttcctcctccttggcggttgatgccaataactgcagcggctgaagcgatttgggcgtgttcgattgaagaactagggctggagaatgaggccgagaaaccgagcaatgcgttgttaactagagcttggtctccaggatggagcaatgctgataagttactaaatgagttcatcgagaagcagttgatagattatgcaaagaacagcaagaaagttgttgggaattctacttcactactttctccgtatctccatttcggggaaataagcgtcagacacgttttccagtgtgcccggatgaaacaaattatatgggcaagagataagaacagtgaaggagaagaaagtgcagatctttttcttaggggaatcggtttaagagagtattctcggtatatatgtttcaacttcccgtttactcacgagcaatcgttgttgagtcatcttcggtttttcccttgggatgctgatgttgataagttcaaggcctggagacaaggcaggaccggttatccgttggtggatgccggaatgagagagttttgggctaccggatggatgcataacagaataagagtgattgtttcaagctttgctgtgaagtttcttctccttccatggaaatggggaatgaagtatttctgggatacacttttggatgctgatttggaatgtgacatccttggctggcagtatatctctgggagtatccccgatggccacgagcttgatcgcttggacaatcccgcgttacaaggcgccaaatatgacccagaaggtgagtacataaggcaatggcttcccgagcttgcgagattgccaactgaatggatccatcatccatgggacgctcctttaaccgtactcaaagcttctggtgtggaactcggaacaaactatgcgaaacccattgtagacatcgacacagctcgtgagctactagctaaagctatttcaagaacccgtgaagcacagatcatgatcggagcagcacctgatgagattgtagcagatagcttcgaggccttaggggctaataccattaaagaacctggtctttgcccatctgtgtcttctaatgaccaacaagtaccttcggctgttggtggcggtggctctggaggtggtgggtccggaggaggcggccgcacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgtaattaattaactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttatacgcgtgccaccatgatgacagacataaaagatggaccacgctcaggggaagatgtatcagctgcaagatctttccctggttccaaaggaatgaacttacctgctagcaagtctgtggcctctggaattctgcctgatgacagttacagaatggattatccagagataggagtatgtgttataataaacaataagaacttccaccgagataccggactgtcatctcgttcaggcacggatgcagatgctgcaagtgtcagagaagtttttatgaagctgggatataaagtcaagcttaacaatgatctgtcaagcagagatatttttaagctattgaaaaatgtttctgaagaagatcacagcaagcgaagcagttttgtttgtgtgttgctaagccatggcgatgaaggactcttctatggtacagatggccctcttgaactgaaagtactaaccagccttttcagaggtgacaagtgcagaagtctagcagggaaacccaaactctttttcattcaggcctgtagaggaacagaattagattctggtattgaagcagccagtggaccagatgaaacagtgtgtcaaaaaatacctgtagaagcagacttcctgtatgcatattctactgctccaggctactactcctggaggaacgcagctgaaggctcctggtttattcagtctctgtgtaggatgctgaaggaacacgccaggaaacttgaactcatgcagattttaactcgtgtaaatcgcagagtggcagaatatgaatcctgctccactcgacaggatttcaatgcaaagaaacagattccatgcattgagtctatgcttaccaaagaattctactttccttgctaaacgcgtactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgccgtttccacattcttttctcatccttcttctcctgttttctctgcatcaaggtcagcacgatagcactgtctctctatgcttagggagaggcctgtcctgtacatcccgtgcccccacaagatgcctactacaacaacatcttctgcatgtcctgcatagcagtgttgggagaatgtgcactacttccactcttctgatttctattttatgtgtttgctttataccagtgttgccatttgggaattaatacatggttgatcaaatcaattgcatcacagctgtatcctgtatcagaggaacattatcaaagcttttgttgctgtatttggtatctgacctgcagataaacatgttttaggaaggttttgcaaaagtagctgtgaaatgagctggtgttgtgatttaacctgacaggcagctaaacagtataccacagagctattcacctactttccctcagtgggaaaagggaagagaactgaggggggggggaataaataagtaaataacaaaataaaactcatggattaagaaaaagactttgtactggaatggatgagaagaataatagtaatgataataatatgtcactctgaaagtaatgcctcttatttctgtggagactacaaacatacaaagagcacaacattccatagagcaaattctcagttacagaatgctattttttttttcaacacagtcaaaatcattaatttttttttgcctgcaatggacaagagctttgaagctgttctcgtaaaaatctgtactagcagaagtgacctgcaatcactactgctgaaatgcacaacccaccacatcattgtgctcacattcactgtttggtttctgtaaatgtacaggaattgtctgaaattagatatgattttttttttctccatgaaggaattcaattacacacctttgcctcatgcacttctttgtcatttttgtcagactgcttctctcctgcaatttgtctcatggcaacaaaatataatggagttctgctgggaacttccctactgccataccactatcatctgcctctgacattttggacaaatgtaataaaataggaggtattactttcagagcagaccttgtatgtatttacaaaacaagtggtacacaaaaaaaattgttcatcccaccaaccaatgcccatcctgtccctgaatagtagctgtcccccacagccttgaccagtttaggtcaacagttctgcttctgtcccctcccagctccttgtaacccctcagccccccttgctggcaggacagtatgagaagctgaaaaactagaatgtcctagttctttgcagtgctgctaatcaacaaccaaaacagtggtgtgttaccaatattgttgatatcacagcatcataccattatgaaggaagtaacccagccaaaatcaggtcagcttgctaacaagagaactgtgcataagtttaagatgtgtgtgttcctcagtaccttaaaaaataagtagtaacgttcaaatgagtagaagagtagaactgagcttaaaacatctgtcagacaacagtgaaccaacc

SEQ ID NO: 127 - DNA, 11629 bp, synthetic construct
  misc_feature 1..11629: note = Targeting Vector TV8
  source 1..11629: mol_type = other DNA
gaatgaagggcctgagggtgggcagtctgtctatcatgtacatctccatattctgggaggtcgtcagttgggctggcctcctggctaagatttttgcaccacaagagatgctgcatgtgtacaaatcactagcaaatagatttgtttcccatcaacttagccactgttaatgtaaattgttcttggatatgtgtctttggagggcaataaatgctctgaacagcacttgcacaataaagatacagcatgtgggaatgatctgtctcatgtgtcttactgatggtattggttctgtaagataaaatattgtgtctgggatgtgtttggctctactattaatggtgctctattgattgtgatttgtcatttgaaacctgaggatgcgactgtatagcagtctttcatgcatttttggaaaaaaacttaagctttttgaaagctgctgctacaactttttgtattgttataaagttttgtattgtttttttaattgtgaaattataaagatgccgtgcagggactgtttgaagcaaagtgcattgttttagaaacctacaactctagttcaagcactccatcagtatctgcttaatctttgtcatcctttgctatgagaaaatattaagcagtagtctaaaggtactatgaaactataacatagctgacattgtatttataactacgtcatgattttgatagaattgaggacttgaaaatgttaaactattcatgtagggcctcttaagatgcttaagttgtttagtaatgtaagtgtgcatttaattgagattttattgggcataatttgtccatcagtatgacactccttgtcagtgttgccttatacttgatgttgttaccggatctctgcaaggcagttattcttgaattaggctcattgaagtgtctgccagtataaatatatagcaactgttctttgtgttaaaattgagaagctaaccagtttttagtgcttctgactgttggaattctttaagcagatgccataagaaaattgtatttgtgatcaccacttctccagagtggttttaacaccaagggcattagagaaagaaaggcaggcgtgtagagaatagtggacagacaaaagctgtgagttacgttatgtttttcagctgaaaagctgtgtttggtaaaagcatatgaaatcactcaacttggaagcattctcttagttctctgatagttctgagcagcagaactcttcacctaagaggttacttcaactggaagactacctagtgcttctgatggcaactatatttaagatgagaataagaggtgtttccagtgtggtagcctcacatctgttgcagtggttaccgttcgtcctcctccgagggacacagcttggccattcactgtggtgacaccaatatgatgatcagcaaatggtgtttattcactactaaacacagcttatatacatttttacctacaaaatcgtgctgtcatgtcccactctgattggttcacacatttccccgaaaagtgccacctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttaataacttcgtataatgtatgctatacgaagttataccggtgaagttcctattccgaagttcctattctctagaaagtataggaacttcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcagcggcgccaccaacttcagcctgctgaagcaggccggcgacgtggaggagaaccccggccccgctagctaaagcggccgccaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagaagttcctattccgaagttcctattctctagaaagtataggaacttcgctagcaccggtgccaccatgaatggagctataggaggtgaccttttgctcaattttcctgacatgtcggtcctagagcgccaaagggctcacctcaagtacctcaatcccacctttgattctcctctcgccggcttctttgccgattcttcaatgattaccggcggcgagatggacagctatctttcgactgccggtttgaatcttccgatgatgtacggtgagacgacggtggaaggtgattcaagactctcaatttcgccggaaacgacgcttgggactggaaatttcaagaaacggaagtttgatacagagactaaggattgtaatgagaagaagaagaagatgacgatgaacagagatgacctagtagaagaaggagaagaagagaagtcgaaaataacagagcaaaacaatgggagcacaaaaagcatcaagaagatgaaacacaaagccaagaaagaagagaacaatttctctaatgattcatctaaagtgacgaaggaattggagaaaacggattatattcatggtggcggtggctctggaggtggtgggtccggaggaggcggccgccgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagcccgggtaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgcctaaaaagaagcgtaaagtcatgaagatgaagatggacaaaaagactatagtttggtttagaagagacctaaggattgaggataatcctgcattagcagcagctgctcacgaaggatctgtttttcctgtcttcatttggtgtcctgaagaagaaggacagttttatcctggaagagcttcaagatggtggatgaaacaatcacttgctcacttatctcaatccttgaaggctcttggatctgacctcactttaatcaaaacccacaacacgatttcagcgatcttggattgtatccgcgttaccggtgctacaaaagtcgtctttaaccacctctatgatcctgtttcgttagttcgggaccataccgtaaaggagaagctggtggaacgtgggatctctgtgcaaagctacaatggagatctattgtatgaaccgtgggagatatactgcgaaaagggcaaaccttttacgagtttcaattcttactggaagaaatgcttagatatgtcgattgaatccgttatgcttcctcctccttggcggttgatgccaataactgcagcggctgaagcgatttgggcgtgttcgattgaagaactagggctggagaatgaggccgagaaaccgagcaatgcgttgttaactagagcttggtctccaggatggagcaatgctgataagttactaaatgagttcatcgagaagcagttgatagattatgcaaagaacagcaagaaagttgttgggaattctacttcactactttctccgtatctccatttcggggaaataagcgtcagacacgttttccagtgtgcccggatgaaacaaattatatgggcaagagataagaacagtgaaggagaagaaagtgcagatctttttcttaggggaatcggtttaagagagtattctcggtatatatgtttcaacttcccgtttactcacgagcaatcgttgttgagtcatcttcggtttttcccttgggatgctgatgttgataagttcaaggcctggagacaaggcaggaccggttatccgttggtggatgccggaatgagagagttttgggctaccggatggatgcataacagaataagagtgattgtttcaagctttgctgtgaagtttcttctccttccatggaaatggggaatgaagtatttctgggatacacttttggatgctgatttggaatgtgacatccttggctggcagtatatctctgggagtatccccgatggccacgagcttgatcgcttggacaatcccgcgttacaaggcgccaaatatgacccagaaggtgagtacataaggcaatggcttcccgagcttgcgagattgccaactgaatggatccatcatccatgggacgctcctttaaccgtactcaaagcttctggtgtggaactcggaacaaactatgcgaaacccattgtagacatcgacacagctcgtgagctactagctaaagctatttcaagaacccgtgaagcacagatcatgatcggagcagcacctgatgagattgtagcagatagcttcgaggccttaggggctaataccattaaagaacctggtctttgcccatctgtgtcttctaatgaccaacaagtaccttcggctgttggtggcggtggctctggaggtggtgggtccggaggaggcggccgcacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgtaattaattaactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttatacgcgtgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgtggcggctagacgcgtactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgccgtttccacattcttttctcatccttcttctcctgttttctctgcatcaaggtcagcacgatagcactgtctctctatgcttagggagaggcctgtcctgtacatcccgtgcccccacaagatgcctactacaacaacatcttctgcatgtcctgcatagcagtgttgggagaatgtgcactacttccactcttctgatttctattttatgtgtttgctttataccagtgttgccatttgggaattaatacatggttgatcaaatcaattgcatcacagctgtatcctgtatcagaggaacattatcaaagcttttgttgctgtatttggtatctgacctgcagataaacatgttttaggaaggttttgcaaaagtagctgtgaaatgagctggtgttgtgatttaacctgacaggcagctaaacagtataccacagagctattcacctactttccctcagtgggaaaagggaagagaactgaggggggggggaataaataagtaaataacaaaataaaactcatggattaagaaaaagactttgtactggaatggatgagaagaataatagtaatgataataatatgtcactctgaaagtaatgcctcttatttctgtggagactacaaacatacaaagagcacaacattccatagagcaaattctcagttacagaatgctattttttttttcaacacagtcaaaatcattaatttttttttgcctgcaatggacaagagctttgaagctgttctcgtaaaaatctgtactagcagaagtgacctgcaatcactactgctgaaatgcacaacccaccacatcattgtgctcacattcactgtttggtttctgtaaatgtacaggaattgtctgaaattagatatgattttttttttctccatgaaggaattcaattacacacctttgcctcatgcacttctttgtcatttttgtcagactgcttctctcctgcaatttgtctcatggcaacaaaatataatggagttctgctgggaacttccctactgccataccactatcatctgcctctgacattttggacaaatgtaataaaataggaggtattactttcagagcagaccttgtatgtatttacaaaacaagtggtacacaaaaaaaattgttcatcccaccaaccaatgcccatcctgtccctgaatagtagctgtcccccacagccttgaccagtttaggtcaacagttctgcttctgtcccctcccagctccttgtaacccctcagccccccttgctggcaggacagtatgagaagctgaaaaactagaatgtcctagttctttgcagtgctgctaatcaacaaccaaaacagtggtgtgttaccaatattgttgatatcacagcatcataccattatgaaggaagtaacccagccaaaatcaggtcagcttgctaacaagagaactgtgcataagtttaagatgtgtgtgttcctcagtaccttaaaaaataagtagtaacgttcaaatgagtagaagagtagaactgagcttaaaacatctgtcagacaacagtgaaccaacc

SEQ ID NO: 128 - DNA, 5894 bp, synthetic construct
  misc_feature 1..5894: note = Plasmid pCAGG-Cre
  source 1..5894: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcggcttgatcgaagcttgcccaccatggcacccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttgtgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctgcaagatggcgattagaagctttcgatcaagccgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattg

SEQ ID NO: 129 - DNA, 6086 bp, synthetic construct
  misc_feature 1..6086: note = Plasmid pCAGG-FlpO
  source 1..6086: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggcggccgccaccatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctgatgaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattg

SEQ ID NO: 130 - DNA, 6842 bp, synthetic construct
  misc_feature 1..6842: note = Plasmid pCAGG-Noggin-IRES-GFP
  source 1..6842: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagcacctgcacatgggacgtcgacctgaggtaattataacccgggatggatcattcccagtgccttgtgactatatacgccgcggcggtgctgctggggctccggctgcagcagggctcctgccagcactacctgcacatccgcccggctcccagcgacaacctgcccctggtggatctaatcgagcacccggaccctatctttgaccccaaggagaaggatcttaacgagaccttgctaaggagcctcatgggaggacacttcgaccctaactttatggctatgtccctgcccgaggaccggctcggggtagacgatctggccgagctggacttgctgctgcggcagagaccctcgggagcgatgcccggcgaaatcaaggggctggagttctacgacgggctgcagccgggcaagaagcacaggctgagcaagaagctgcgcaggaagctgcagatgtggctctggtcccagaccttctgcccggtcctatacacgtggaacgatctcggcagccgcttttggccccggtacgtcaaagtgggcagctgctacagtaaaaggtcttgctctgtcccagaaggcatggtctgcaaacctgccaagtccgtgcatttaacgatcctgaggtggcggtgccagcggcggggcgggcagcggtgcacgtggatccccatccagtaccccatcatcgcggagtgcaagtgctcctgctaggctagcctccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattg

SEQ ID NO: 131 - DNA, 7999 bp, synthetic construct
  misc_feature 1..7999: note = TV-FlpO-IRES-GFP
  source 1..7999: mol_type = other DNA
gaatgaagggcctgagggtgggcagtctgtctatcatgtacatctccatattctgggaggtcgtcagttgggctggcctcctggctaagatttttgcaccacaagagatgctgcatgtgtacaaatcactagcaaatagatttgtttcccatcaacttagccactgttaatgtaaattgttcttggatatgtgtctttggagggcaataaatgctctgaacagcacttgcacaataaagatacagcatgtgggaatgatctgtctcatgtgtcttactgatggtattggttctgtaagataaaatattgtgtctgggatgtgtttggctctactattaatggtgctctattgattgtgatttgtcatttgaaacctgaggatgcgactgtatagcagtctttcatgcatttttggaaaaaaacttaagctttttgaaagctgctgctacaactttttgtattgttataaagttttgtattgtttttttaattgtgaaattataaagatgccgtgcagggactgtttgaagcaaagtgcattgttttagaaacctacaactctagttcaagcactccatcagtatctgcttaatctttgtcatcctttgctatgagaaaatattaagcagtagtctaaaggtactatgaaactataacatagctgacattgtatttataactacgtcatgattttgatagaattgaggacttgaaaatgttaaactattcatgtagggcctcttaagatgcttaagttgtttagtaatgtaagtgtgcatttaattgagattttattgggcataatttgtccatcagtatgacactccttgtcagtgttgccttatacttgatgttgttaccggatctctgcaaggcagttattcttgaattaggctcattgaagtgtctgccagtataaatatatagcaactgttctttgtgttaaaattgagaagctaaccagtttttagtgcttctgactgttggaattctttaagcagatgccataagaaaattgtatttgtgatcaccacttctccagagtggttttaacaccaagggcattagagaaagaaaggcaggcgtgtagagaatagtggacagacaaaagctgtgagttacgttatgtttttcagctgaaaagctgtgtttggtaaaagcatatgaaatcactcaacttggaagcattctcttagttctctgatagttctgagcagcagaactcttcacctaagaggttacttcaactggaagactacctagtgcttctgatggcaactatatttaagatgagaataagaggtgtttccagtgtggtagcctcacatctgttgcagtggttaccgttcgtcctcctccgagggacacagcttggccattcactgtggtgacaccaatatgatgatcagcaaatggtgtttattcactactaaacacagcttatatacatttttacctacaaaatcgtgctgtcatgtcccactctgattggttcactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagcacctgcacatgggacgtcgacctgaggtaattataacccggggccaccatggctcctaagaagaagaggaaggtgatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctgagctagccgatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcatgccgtttccacattcttttctcatccttcttctcctgttttctctgcatcaaggtcagcacgatagcactgtctctctatgcttagggagaggcctgtcctgtacatcccgtgcccccacaagatgcctactacaacaacatcttctgcatgtcctgcatagcagtgttgggagaatgtgcactacttccactcttctgatttctattttatgtgtttgctttataccagtgttgccatttgggaattaatacatggttgatcaaatcaattgcatcacagctgtatcctgtatcagaggaacattatcaaagcttttgttgctgtatttggtatctgacctgcagataaacatgttttaggaaggttttgcaaaagtagctgtgaaatgagctggtgttgtgatttaacctgacaggcagctaaacagtataccacagagctattcacctactttccctcagtgggaaaagggaagagaactgaggggggggggaataaataagtaaataacaaaataaaactcatggattaagaaaaagactttgtactggaatggatgagaagaataatagtaatgataataatatgtcactctgaaagtaatgcctcttatttctgtggagactacaaacatacaaagagcacaacattccatagagcaaattctcagttacagaatgctattttttttttcaacacagtcaaaatcattaatttttttttgcctgcaatggacaagagctttgaagctgttctcgtaaaaatctgtactagcagaagtgacctgcaatcactactgctgaaatgcacaacccaccacatcattgtgctcacattcactgtttggtttctgtaaatgtacaggaattgtctgaaattagatatgattttttttttctccatgaaggaattcaattacacacctttgcctcatgcacttctttgtcatttttgtcagactgcttctctcctgcaatttgtctcatggcaacaaaatataatggagttctgctgggaacttccctactgccataccactatcatctgcctctgacattttggacaaatgtaataaaataggaggtattactttcagagcagaccttgtatgtatttacaaaacaagtggtacacaaaaaaaattgttcatcccaccaaccaatgcccatcctgtccctgaatagtagctgtcccccacagccttgaccagtttaggtcaacagttctgcttctgtcccctcccagctccttgtaacccctcagccccccttgctggcaggacagtatgagaagctgaaaaactagaatgtcctagttctttgcagtgctgctaatcaacaaccaaaacagtggtgtgttaccaatattgttgatatcacagcatcataccattatgaaggaagtaacccagccaaaatcaggtcagcttgctaacaagagaactgtgcataagtttaagatgtgtgtgttcctcagtaccttaaaaaataagtagtaacgttcaaatgagtagaagagtagaactgagcttaaaacatctgtcagacaacagtgaaccaacc

SEQ ID NO: 132 - DNA, 7263 bp, synthetic construct
  misc_feature 1..7263: note = TV-Cre-P2A-GFP
  source 1..7263: mol_type = other DNA
gaatgaagggcctgagggtgggcagtctgtctatcatgtacatctccatattctgggaggtcgtcagttgggctggcctcctggctaagatttttgcaccacaagagatgctgcatgtgtacaaatcactagcaaatagatttgtttcccatcaacttagccactgttaatgtaaattgttcttggatatgtgtctttggagggcaataaatgctctgaacagcacttgcacaataaagatacagcatgtgggaatgatctgtctcatgtgtcttactgatggtattggttctgtaagataaaatattgtgtctgggatgtgtttggctctactattaatggtgctctattgattgtgatttgtcatttgaaacctgaggatgcgactgtatagcagtctttcatgcatttttggaaaaaaacttaagctttttgaaagctgctgctacaactttttgtattgttataaagttttgtattgtttttttaattgtgaaattataaagatgccgtgcagggactgtttgaagcaaagtgcattgttttagaaacctacaactctagttcaagcactccatcagtatctgcttaatctttgtcatcctttgctatgagaaaatattaagcagtagtctaaaggtactatgaaactataacatagctgacattgtatttataactacgtcatgattttgatagaattgaggacttgaaaatgttaaactattcatgtagggcctcttaagatgcttaagttgtttagtaatgtaagtgtgcatttaattgagattttattgggcataatttgtccatcagtatgacactccttgtcagtgttgccttatacttgatgttgttaccggatctctgcaaggcagttattcttgaattaggctcattgaagtgtctgccagtataaatatatagcaactgttctttgtgttaaaattgagaagctaaccagtttttagtgcttctgactgttggaattctttaagcagatgccataagaaaattgtatttgtgatcaccacttctccagagtggttttaacaccaagggcattagagaaagaaaggcaggcgtgtagagaatagtggacagacaaaagctgtgagttacgttatgtttttcagctgaaaagctgtgtttggtaaaagcatatgaaatcactcaacttggaagcattctcttagttctctgatagttctgagcagcagaactcttcacctaagaggttacttcaactggaagactacctagtgcttctgatggcaactatatttaagatgagaataagaggtgtttccagtgtggtagcctcacatctgttgcagtggttaccgttcgtcctcctccgagggacacagcttggccattcactgtggtgacaccaatatgatgatcagcaaatggtgtttattcactactaaacacagcttatatacatttttacctacaaaatcgtgctgtcatgtcccactctgattggttcattaattaagaagttcctattccgaagttcctattctctagaaagtataggaacttcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttaggtaccatggcacccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttgtgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctgcaagatggcgatcccgggggaagcggagctactaacttcagcctgctgaagcaggctggcgacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgaagttcctattccgaagttcctattctctagaaagtataggaacttcgctagcgccgtttccacattcttttctcatccttcttctcctgttttctctgcatcaaggtcagcacgatagcactgtctctctatgcttagggagaggcctgtcctgtacatcccgtgcccccacaagatgcctactacaacaacatcttctgcatgtcctgcatagcagtgttgggagaatgtgcactacttccactcttctgatttctattttatgtgtttgctttataccagtgttgccatttgggaattaatacatggttgatcaaatcaattgcatcacagctgtatcctgtatcagaggaacattatcaaagcttttgttgctgtatttggtatctgacctgcagataaacatgttttaggaaggttttgcaaaagtagctgtgaaatgagctggtgttgtgatttaacctgacaggcagctaaacagtataccacagagctattcacctactttccctcagtgggaaaagggaagagaactgaggggggggggaataaataagtaaataacaaaataaaactcatggattaagaaaaagactttgtactggaatggatgagaagaataatagtaatgataataatatgtcactctgaaagtaatgcctcttatttctgtggagactacaaacatacaaagagcacaacattccatagagcaaattctcagttacagaatgctattttttttttcaacacagtcaaaatcattaatttttttttgcctgcaatggacaagagctttgaagctgttctcgtaaaaatctgtactagcagaagtgacctgcaatcactactgctgaaatgcacaacccaccacatcattgtgctcacattcactgtttggtttctgtaaatgtacaggaattgtctgaaattagatatgattttttttttctccatgaaggaattcaattacacacctttgcctcatgcacttctttgtcatttttgtcagactgcttctctcctgcaatttgtctcatggcaacaaaatataatggagttctgctgggaacttccctactgccataccactatcatctgcctctgacattttggacaaatgtaataaaataggaggtattactttcagagcagaccttgtatgtatttacaaaacaagtggtacacaaaaaaaattgttcatcccaccaaccaatgcccatcctgtccctgaatagtagctgtcccccacagccttgaccagtttaggtcaacagttctgcttctgtcccctcccagctccttgtaacccctcagccccccttgctggcaggacagtatgagaagctgaaaaactagaatgtcctagttctttgcagtgctgctaatcaacaaccaaaacagtggtgtgttaccaatattgttgatatcacagcatcataccattatgaaggaagtaacccagccaaaatcaggtcagcttgctaacaagagaactgtgcataagtttaagatgtgtgtgttcctcagtaccttaaaaaataagtagtaacgttcaaatgagtagaagagtagaactgagcttaaaacatctgtcagacaacagtgaaccaacc